(19) United States
(12) Patent Application Publication
(10) Pub. No.: US 2009/0069180 A1
(43) Pub. Date: Mar. 12, 2009

(54) 2-SUBSTITUTED PYRIMIDINE DERIVATIVES

(76) Inventors: Joachim Rheinheimer, Ludwigshafen (DE); Thomas Grote, Wachenheim (DE); Bernd Muller, Frankenthal (DE); Wassilios Grammenos, Ludwigshafen (DE); Sarah Ulmschneider, Bad Durkheim (DE); Jens Renner, Bad Durkheim (DE); Reinhard Stierl, Kaohsiung County (TW)

Correspondence Address: BRINKS, HOFER, GILSON & LIONE, P.O. BOX 1340, MORRISVILLE, NC, 27560, US

(21) Appl. No.: 12/279,298

(22) PCT Filed: Feb. 7, 2007
(86) PCT No.: PCT/EP2007/051142

§ 371 Date: Aug. 13, 2008

(30) Foreign Application Priority Data
Feb. 15, 2006 (EP) 06003084.8

(51) Int. Cl. A01N 25/26; A01N 43/54; C07D 403/02

(52) U.S. Cl. 504/100; 514/256; 544/333

(57) ABSTRACT

The present invention relates to the use of 2-substituted pyrimidines of the formula I

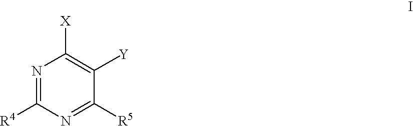

in which the substituents have the meanings as defined in the description, as fungicides, to novel pyrimidines, to processes and intermediates for producing them, and to compositions comprising them.

The present invention relates to the use of pyrimidines of the formula I

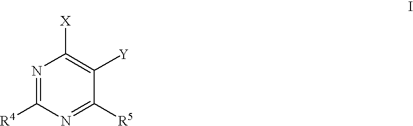

in which the substituents are as defined below:
X is NR¹R², OR³, SR³ or C₁-C₈-alkyl which may contain one, two, three or four identical or different substituents R^{a};
   where R¹, R², R³ are as defined below:
   R¹, R² independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₄-C₈-cycloalkenyl, C₄-C₈-halocycloalkenyl, C₄-C₁₀-alkadienyl, C₄-C₁₀-haloalkadienyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkoxy, C₃-C₈-halocycloalkoxy, C₅-C₁₀-bicycloalkyl, NH₂, C₁-C₈-alkylamino, di-C₁-C₈-alkylamino, phenyl, naphthyl or a five- to six-membered saturated, partially unsaturated or aromatic heterocycle which contains one, two, three or four heteroatoms from the group consisting of O, N and S,
      R¹ and R² together with the nitrogen atom to which they are attached may also form a five-, six- or seven-membered saturated or partially unsaturated heterocycle which may contain one, two or three further heteroatoms from the group consisting of O, N and S as ring members, where
      R¹ and/or R² or a heterocycle formed by R¹ and R² may carry one, two, three or four identical or different groups R^{a} and/or two substituents attached to the same or adjacent atoms or ring atoms may be C₁-C₆-alkylene, oxy-C₂-C₄-alkylene or oxy-C₁-C₃-alkyleneoxy;
   R³ is C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₁-C₈-alkylamino, di-C₁-C₈-alkylamino, where R³ may carry one, two, three or four identical or different groups R^{a};
      where R^{a} is as defined below:
      R^{a} is halogen, cyano, nitro, hydroxy, carboxyl, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₄-C₁₀-alkadienyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₃-C₈-cycloalkenyloxy, C₅-C₁₀-bicycloalkyl, C₁-C₈-alkylcarbonyloxy, C₁-C₈-alkoxycarbonyloxy, aminocarbonyloxy, C₁-C₈-alkylaminocarbonyloxy, di-C₁-C₈-alkylaminocarbonyloxy, C₁-C₈-alkylthiocarbonyloxy, C₁-C₈-alkoxythiocarbonyloxy, aminothiocarbonyloxy, C₁-C₈-alkylaminothiocarbonyloxy, di-C₁-C₈-alkylaminothiocarbonyloxy C₁-C₈-alkylaminothiocarbonyl, di-C₁-C₈-alkylaminothiocarbonyl, C₁-C₆-alkylene, oxy-C₂-C₄-alkylene, oxy-C₁-C₃-alkyleneoxy, ═CH₂, ═CH(C₁-C₄-alkyl), ═C(C₁-C₄-alkyl)₂, —C(═O)-A, —C(═O)—O-A, —C(═O)—N(A′)A, C(A′)(=N—OA), N(A′)A, N(A′)-C(═O)-A, N(A″)—C(═O)—N(A′)A, S(═O)ₘ-A, S(═O)ₘ—O-A, S(═O)ₘ—N(A′)A, —Si(C₁-C₆-alkyl)₃, phenyl, naphthyl, a five-, six-, seven-, eight-, nine- or ten-membered saturated, partially unsaturated or aromatic heterocycle which contains one, two, three or four heteroatoms from the group consisting of O, N and S;
      where the aliphatic, alicyclic and/or aromatic groups in R^{a} for their part may carry one, two or three identical or different groups R^{b}:
      R^{b} is halogen, cyano, nitro, hydroxy, mercapto, C₁-C₈-alkyl, C₂-C₈-alkenyl, C₂-C₈-alkynyl, C₄-C₁₀-alkadienyl, C₃-C₈-cycloalkyl, C₃-C₈-cycloalkenyl, C₅-C₁₀-bicycloalkyl, C₁-C₈-alkoxy, C₂-C₈-alkenyloxy, C₂-C₈-alkynyloxy, C₃-C₈-cycloalkoxy, C₃-C₈-cycloalkenyloxy, aminothiocarbonyl, C₁-C₈-alkylcarbonyloxy, C₁-C₈-alkylaminothiocarbonyl, di-C₁-C₈-alkylaminothiocarbonyl, C₁-C₈-alkylthio, C₁-C₈-alkylsulfinyl, C₁-C₈-alkylsulfonyl, C₆-C₁₄-aryl, C₆-C₁₄-aryloxy, C₆-C₁₄-arylthio, C₆-C₁₄-aryl-C₁-C₆-alkoxy, C₆-C₁₄-arylC₁-C₆-alkyl, a five- to ten-membered saturated, partially unsaturated or aromatic heterocycle which contains one, two, three or four heteroatoms from the group consisting of O, N and S, heterocyclyloxy, heteroaryloxy, heteroarylthio, —C(═O)-A, —C(—O)—O-A, —C(═O)—N(A′)A, C(A′)(=N—OA), N(A′)A; where the aliphatic, alicyclic or aromatic groups may be partially or fully halogenated and/or substituted by C₁-C₈-alkyl and/or C₁-C₈-haloalkyl groups;
      where m, A, A′ and A″ are as defined below:
         m is 0, 1 or 2;
         A, A′, A″ independently of one another are hydrogen, C₁-C₈-alkyl, C₂-C₈-alkenyl, C₂-C₈-alkynyl, C₃-C₈-cycloalkyl, C₃-C₈-cycloalkenyl, phenyl, where the organic radicals may be partially or fully halogenated and/or may be mono- or polysubstituted by nitro, cyanato, cyano, C₁-C₄-alkoxy; A and A′ together with the atoms to which they are attached may also be a five- or six-membered saturated, partially unsaturated or aromatic heterocycle which contains one, two, three or four heteroatoms from the group consisting of O, N and S;
Y is (C₂-C₁₀)-alkenyl, (C₂-C₁₀)-alkynyl, (C₃-C₁₀)-cycloalkenyl, a five-, six-, seven-, eight-, nine- or ten-membered partially unsaturated heterocycle which contains one, two or three heteroatoms from the group consisting of O, N and S as ring members, or an imino group; where Y may carry one, two, three or four identical or different groups R^{u} and/or two substituents attached to the same or adjacent atoms or ring atoms are C₁-C₆-alkylene, oxy-C₂-C₄-alkylene or oxy-C₁-C₃-alkyleneoxy, where R^{u} is as defined below:
   R^{u} independently of one another are halogen, cyano, nitro, C₁-C₈-alkyl, C₁-C₈-haloalkyl, C₁-C₈-alkoxy-C₁-C₈-alkyl, C₂-C₈-alkenyl, C₂-C₈-haloalkenyl, C₂-C₈-alkynyl, C₂-C₈-haloalkynyl, C₄-C₁₀-alkadienyl, C₃-C₆-cycloalkyl, C₃-C₆-halocycloalkyl, C₄-C₆-cycloalkenyl, C₄-C₆-halocycloalkenyl, C₅-C₁₀-bicycloalkyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkoxy, C₃-C₈-halocycloalkoxy, C₃-C₈-cycloalkenyloxy, C₁-C₈-alkylcarbonyloxy, C₁-C₈-alkylaminothiocarbonyl, di-C₁-C₈-alkylaminothiocarbonyl, C₁-C₆-alkylene, oxy-C₂-C₄-alkylene, oxy-C₁-C₃-alkyleneoxy, —C(═O)-A, —C(═O)—O-A, —C(═O)—N(A′)A, C(A′)(=N—OA), N(A′)A, N(A′)C(═O)-A, N(A″)—C(═O)—N(A′)A, S(═O)ₘ-A, S(═O)ₘ—O-A, S(═O)ₘ—N(A′)A, —Si(C₁-C₆-alkyl)₃, phenyl, naphthyl, a five- to ten-membered saturated, partially unsaturated or aromatic heterocycle which contains one, two, three or four heteroatoms from the group consisting of O, N and S; where m, A, A′, A″ are as defined above;
   R^{u} may carry one, two, three or four identical or different groups R^{a}, where R^{a} is as defined above;
R⁴ is a five- or six-membered saturated, partially unsaturated or aromatic heterocycle which contains one, two, three or four heteroatoms from the group consisting of O, N and S, where R⁴ may be partially or fully halogenated and/or may carry one, two, three or four identical or different groups R^{v}:
   R^{v} is halogen, cyano, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₁-C₆-alkoxy, C₁-C₆-haloalkoxy, C₃-C₆-cycloalkyl, C₃-C₆-halocycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, Oxo (═O), —C(═O)A, —C(═O)—O-A, —C(═O)—N(A′)A, —C(A′)(=N—OA), N(A′)A, N(A′)-C(═O)-A, N(A″-C(═O)—N(A′)A, —S(═O)ₘ-A, —S(═O)ₘ—O-A or —S(═O)ₘ—N(A′)A; where m, A, A′, A″ are as defined above;
   R⁴ may furthermore be:
   cyano, C(═W)OR^{c}, C(═W)NR^{z}R^{d}, C(═W)NR^{c}—NR^{z}R^{d}, C(═W)R^{c},
   CR^{c}R^{d}—OR^{z}, CR^{c}R^{d}—NR^{z}R^{f},
   ON(═CR^{c}R^{d}), O—C(═W)R^{c},
   NR^{c}R^{d}, NR^{c}C(═W)R^{d}), NR^{c}C(═W)OR^{d}), NR^{c}C(═W)—NR^{z}R^{d}), NR^{c}(N═CR^{f}R^{d}),
   NR^{c}—NR^{z}R^{d}, NR^{z}—OR^{c}; where
   W is O, S, NR^{g}, NOR^{g} or N—NR^{y}RF;
   R^{c}, R^{d}, R^{f}, R^{g} independently of one another are hydrogen, C₁-C₆-alkyl, C₂-C₆-alkenyl, C₂-C₆-alkynyl, C₃-C₆-cycloalkyl or C₄-C₆-cycloalkenyl;
   R^{y}, R^{z} independently have the same meanings as R^{c} and may additionally be C(═O)—R^{g} or C(═O)—OR^{g};
   where the aliphatic and/or alicyclic groups of the radical definitions of R^{c}, R^{d}, R^{f}, R^{g}, R^{y} and R^{z} for their part may be partially or fully halogenated and/or may carry one, two, three or four identical or different groups R^{w}:
   R^{w} is halogen, cyano, C₁-C₈-alkyl, C₂-C₁₀-alkenyl, C₂-C₁₀-alkynyl, C₃-C₆-cycloalkyl, C₃-C₆-cycloalkenyl, C₁-C₆-alkoxy, C₂-C₁₀-alkenyloxy, C₂-C₁₀-alkynyloxy, C₃-C₆-cycloalkoxy, C₃-C₆-cycloalkenyloxy;
   and where two of the radicals R^{c}, R^{d}, R^{f}, R^{y}, R^{z} together with the atoms to which they are attached may form a five- or six-membered saturated, partially unsaturated or aromatic heterocycle which contains one, two, three or four heteroatoms from the group consisting of O, N and S;
R⁵ is hydrogen, hydroxy, halogen, cyano, azido, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₄-C₈-alkadienyl, C₃-C₈-cycloalkyl, C₃-C₈-halocycloalkyl, C₁-C₈-alkoxy, C₁-C₈-haloalkoxy, C₂-C₈-alkenyloxy, C₃-C₄-alkynyloxy, C₁-C₈-alkylthio, C₁-C₈-alkylsulfinyl, C₁-C₈-alkylsulfonyl, amino, C₁-C₈-alkylamino, di-(C₁-C₈-alkyl)amine, where the aliphatic and alicyclic groups of the radical definitions of R⁵ for their part may contain one, two, three or four substituents independently of one another selected from the group consisting of halogen, cyano, hydroxy, nitro, C₁-C₄-alkoxy, C₁-C₄-alkoxycarbonyl, amino, C₁-C₄-alkylamino and di-C₁-C₄-alkylamino;
   and agriculturally acceptable salts thereof as fungicides.

Furthermore, the present invention relates to novel pyrimidines and the use thereof for controlling phytopathogenic fungi, to a method for controlling phytopathogenic fungi wherein the fungi and/or the materials, plants, the soil and/or the seed to be protected against fungal attack are treated with an effective amount of at least one pyrimidine of the formula I, and to a method for controlling animal pests. The present invention furthermore provides pesticidal compositions comprising at least one of the compounds according to the invention and processes and intermediates for preparing the novel pyrimidines.

Depending on the substitution pattern, the pyrimidines I according to the invention may have one or more centers of chirality, in which case they are present as enantiomer or diastereomer mixtures. The invention provides both the pure enantiomers or diastereomers or rotamers and mixtures thereof. Suitable compounds of the formula I also include all possible stereoisomers (cis/trans isomers) and mixtures thereof. The compounds according to the invention can be present in different crystal modifications, which may differ in their biological activity. They also form part of the subject matter of the present invention.

Substituted pyrimidines having fungicidal action are known from the literature (WO 01/096314, WO 02/074753, WO 03/043993, WO 04/103978). WO 2005/019207 relates to fungicidal pyrimidine derivatives which have a heteroaromatic ring in the 4-position and are substituted in the 5-position by halogen, C₂-C₆-alkenyl or C₂-C₆-alkynyl, which are optionally halogenated.

It was an object of the present invention to provide further compounds having a suitable pesticidal, in particular fungicidal, action. Surprisingly, this object is achieved by the pyrimidines of the formula I.

According to the present invention, agriculturally acceptable salts include in particular the salts of those cations and the acid addition salts of those acids whose cations and anions, respectively, have no adverse effect on the pesticidal action of the pyrimidines according to the invention.

Thus, suitable cations are in particular the ions of the alkali metals, preferably sodium and potassium, of the alkaline earth metals, preferably calcium, magnesium and barium, and of the transition metals, preferably manganese, copper, zinc and iron, and also the ammonium ion which, if desired, may bear from one to four (C₁-C₄)-alkyl substituents and/or one phenyl or benzyl substituent, preferably diisopropylammonium, tetramethylammonium, tetrabutylammonium, trimethylbenzylammonium, and also phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are for example chloride, bromide, fluoride, hydrogen sulfate, sulfate, dihydrogenphosphate, hydrogenphosphate, phosphate, nitrate, bicarbonate, carbonate, hexafluorosilicate, hexafluorophosphate, benzoate, and also the anions of (C₁-C₄)-alkanoic acids, preferably formate, acetate, propionate and butyrate. They can be formed by reacting the compounds according to the invention with an acid of the corresponding anion, preferably hydrochloric acid, hydrobromic acid, sulfuric acid, phosphoric acid or nitric acid.

The compounds according to the invention can be obtained by various routes. They can be prepared, for example, analogously to the syntheses for substituted pyrimidines described in the literature cited at the outset.

Compounds according to the invention can be prepared, for example, from compounds 2

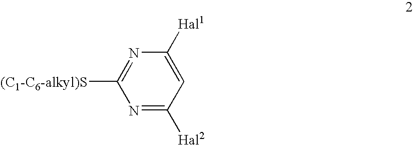

by reacting these with an amine HNR¹R² to give compounds 3.

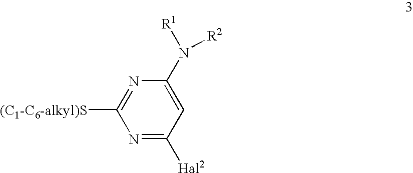

Hal¹ and Hal² are each independently of one another halogen, in particular fluorine, chlorine or bromine, preferably chlorine, and R¹ and R² have the meanings and preferred meanings as defined for formula I. Compounds 2 are known or obtainable analogously to the known substances.

By halogenation in the 5-position, compounds 3 can then be converted further into compounds 4

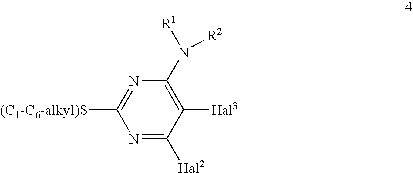

where Hal³ is halogen, in particular fluorine, chlorine or bromine, preferably bromine. Compounds 3 can be brominated, for example, by reaction with bromine (Hal³=Br).

To introduce different radicals R⁴ (as defined for the compounds of the formula I according to the invention), it is posssible, for example, to oxidize the (C₁-C₆-alkylthio) group in the compounds 4 under generally known compounds to the alkylsulfonyl (C₁-C₆-alkyl-S[═O]₂—) group (compound 4a), thus forming a leaving group for further exchange reactions:

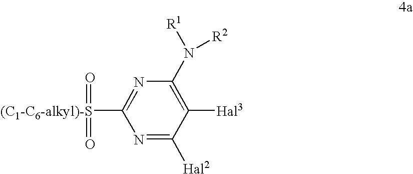

Suitable oxidizing agents are in particular hydrogen peroxide or peracids of organic carboxylic acids; advantageously, MCPBA (meta-chloroperbenzoic acid) may be used. However, the oxidation may also be carried out using selenium dioxide.

By reacting the compounds 4a with a cyanide, such as, for example, potassium cyanide, in the presence of a crown ether (for example 18-crown-6), it is possible to prepare compounds 4b in which R⁴ is thus cyano:

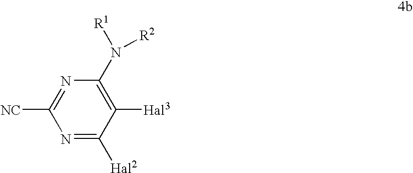

To introduce substituents according to the invention into the 5-position (Y in formula I), the compounds 4b can then be reacted, for example, with trimethylsilylacetylene under conditions generally known to the person skilled in the art, to obtain compounds Ia:

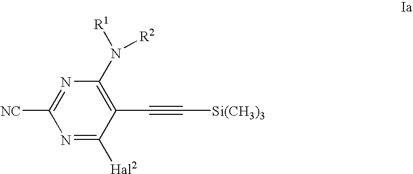

Compounds Ia can then be converted further into compounds of the formula I in which Y is alkenyl. Compounds Ib, for example, can be obtained by reaction with bromine or N-bromosuccinimide (NBS)

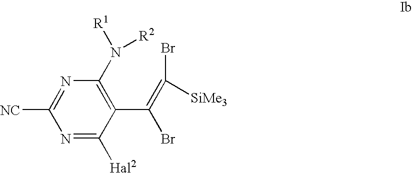

By reaction with, for example, potassium fluoride and N-chlorosuccinimide (NCS), the trimethylsilyl group can be exchanged for Cl:

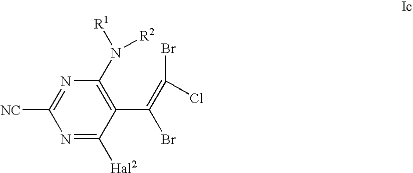

Starting with 2-cyano-substituted pyrimidines of the formula I (R⁴═CN, for example compounds Ia, Ib or Ic) it is possible to prepare 2-amides, 2-amidoximes, 2-amidines by known methods (R⁴ is C(═O)NH₂, C(═NOH)NH₂, C[═NO(C₁-C₆-alkyl)]NH₂, C(═NH)NRCR^{d} (where R^{c} and R^{d} are as defioned above) or C(═NH)NH₂). Here, in the case of the amides, the nitrile is hydrolyzed, for example in the presence of hydrogen peroxide. Amidoximes can be obtained by initially converting the nitrile with methanol and hydrogen chloride or with methanol and sodium methoxide into the imino ester with is then reacted further with a hydroxylamine (R⁴ is C(═N—OH)NH₂) or an O-alkylated hydroxylamine (H₂NO(C₁-C₆-alkyl))- to prepare compounds in which R⁴ is C(═N—OCH₃)NH₂, for example, H₂N—OCH₃ is used. Amidines are formed, for example, directly from the nitrile with ammonia or an amine, or they cab also be prepared from the imino ester by reaction with ammonia or an amine. The reaction can be carried out using processes known in the prior art (see also Cesar, J.; Sollner, M.; Synth. Commun. 2000, 30 (22), 4147-4158).

Depending on the nucleophilicity, to introduce a heterocyclic radical R⁴ the heterocycle can be reacted directly with an alkylsulfonyl compound (compound 4a) (such as, for example, pyrazole, triazole). In these cases, an auxiliary base is generally employed. An azole can be introduced, for example, by initially deprotonating this with a suitable base, such as, for example, an alkali metal alkoxide or hydroxide or sodium hydride, and then reacting it in a suitable solvent, such as, for example, tetrahydrofuran, dioxane or dimethylformamide, with the alkylsulfonyl compound.

However, heterocyclic substituents can also be introduced during the construction of the pyrimidine ring. To this end, a corresponding heterocyclic amidine, which are known to the person skilled in the are or can be prepared from the corresponding heterocyclic nitrites, is reacted with a malonic ester to give the pyrimidine ring (see also WO 2003/070721).

What was stated above also applies to the preparation of compounds in which R⁵ is an alkyl group. An alkyl group (R⁵) can be introduced using organometallic compounds of the formula (R⁵)ₙ-M where M is, for example, magnesium, zinc or lithium, for instance at the stage of the compound 4 or 4a. Here, the use of a transition metal catalyst is frequently advantageous. The use of palladium alkyl- and arylphosphine complexes may give particularly good results. If R⁵ is a cyano group or an alkoxy substituent, the radical R⁵ can be introduced by reaction with alkali metal cyanides and alkali metal alkoxides, respectively.

In the definitions of the symbols given for the compounds according to the invention or the precursors thereof, collective terms were used which are generally representative of the following substituents:

halogen: fluorine, chlorine, bromine and iodine;
alkyl and the alkyl moieties of composite groups such as, for example, alkylamino: saturated straight-chain or branched hydrocarbon radicals having 1 to 2, 4, 6 or 8 carbon atoms, for example C₁-C₆-alkyl, such as methyl, ethyl, propyl, 1-methylethyl, butyl, 1-methyl-propyl, 2-methylpropyl, 1,1-dimethylethyl, pentyl, 1-methylbutyl, 2-methylbutyl, 3-methylbutyl, 2,2-di-methylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: alkyl as mentioned above, where some or all of the hydrogen atoms in these groups are replaced by halogen atoms as mentioned above. In one embodiment, the alkyl groups are substituted at least once or completely by a particular halogen atom, preferably fluorine, chlorine or bromine. In a further embodiment, the alkyl groups are partially or fully halogenated by different halogen atoms; in the case of mixed halogen substitutions, the combination of chlorine and fluorine is preferred. Particular preference is given to (C₁-C₄)-haloalkyl, more preferably (C₁-C₂)-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;
hydroxyalkyl: alkyl as mentioned above where one or more hydrogen atoms are replaced by hydroxyl (OH) groups;
alkenyl and also the alkenyl moieties in composite groups, such as alkenyloxy: unsaturated straight-chain or branched hydrocarbon radicals having 2 to 4, 2 to 6, 2 to 8 or 2 to 10 carbon atoms and one or two, preferably one, double bond in any position. According to the invention, it may be preferred to use small alkenyl groups, such as (C₂-C₄)-alkenyl, on the other hand, it may also be preferred to employ larger alkenyl groups, such as (C₅-C₈)-alkenyl. Examples of alkenyl groups are, for example, C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl—pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
haloalkenyl: alkenyl as defined above, where some or all of the hydrogen atoms in these groups are replaced by halogen atoms as described above under haloalkyl, in particular fluorine, chlorine or bromine;
alkadienyl: unsaturated straight-chain or branched hydrocarbon radicals having 4 to 6, 4 to 8 or 4 to 10 carbon atoms and two double bonds in any position;
alkynyl and the alkynyl moieties in composite groups: straight-chain or branched hydrocarbon groups having 2 to 4, 2 to 6, 2 to 8 or 2 to 10 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
haloalkynyl: alkynyl as defined above, where some or all of the hydrogen atoms in these groups are replaced by halogen atoms as described above under haloalkyl, in particular fluorine, chlorine or bromine;
cycloalkyl and also the cycloalkyl moieties in composite groups: mono- or bicyclic saturated hydrocarbon groups having 3 to 6 or 3 to 8 carbon ring members, for example C₃-C₆-cycloalkyl, such as cyclopropyl, cyclobutyl, cyclopentyl, cyclohexyl;
halocycloalkyl: cycloalkyl as defined above, where some or all of the hydrogen atoms in these groups are replaced by halogen atoms as described above under haloalkyl, in particular fluorine, chlorine or bromine;
cycloalkenyl: monocyclic monounsaturated hydrocarbon groups having preferably 3 to 10, 3 to 8 or 4 to 6, in particular 5 to 6, carbon ring members, such as cyclopenten-1-yl, cyclopenten-3-yl, cyclohexen-1-yl, cyclohexen-3-yl, cyclohexen-4-yi and the like;
halocycloalkenyl: cycloalkenyl as defined above, where some or all of the hydrogen atoms in these groups are replaced by halogen atoms as described above under haloalkyl, in particular fluorine, chlorine or bromine;
alkoxy: an alkyl group as defined above which is attached via an oxygen, preferably having 1 to 8, more preferably 2 to 6, carbon atoms. According to the invention it may be preferred to use small alkoxy groups, such as (C₁-C₄)-alkoxy, on the other hand, it may also be preferred to use larger alkoxy groups, such as (C₅-C₈)-alkoxy. Examples of preferred alkoxy groups are: methoxy, ethoxy, n-propoxy, 1-methylethoxy, butoxy, 1-methylpropoxy, 2-methylpropoxy or 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy;
haloalkoxy: alkoxy as defined above, where some or all of the hydrogen atoms in these groups are replaced by halogen atoms as described above under haloalkyl, in particular fluorine, chlorine or bromine. According to the invention it may be preferred to use short-chain haloalkoxy groups, such as (C₁-C₄)-haloalkoxy, on the other hand, it may also be preferred to use relatively long-chain haloalkoxy groups, such as (C₅-C₈)haloalkoxy. Examples of preferred haloalkoxy radicals are OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy; and also 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy;
alkenyloxy: alkenyl as defined above which is attached via an oxygen atom. Preference is given to (C₂-C₈)-alkenyloxy, more preference to (C₃-C₆)-alkenyloxy. According to the invention, it may be preferred to use short-chain alkenyloxy radicals, such as (C₂-C₄)alkenyloxy, on the other hand, it may also be preferred to use relatively long-chain alkenyloxy groups, such as (C₅-C₈)-alkenyloxy;
alkylene: divalent unbranched chains of CH₂ groups. Preference is given to (C₁-C₆)alkylene, more preference to (C₂-C₄)-alkylene; furthermore, it may be preferred to use (C₁-C₃)-alkylene groups. Examples of preferred alkylene radicals are CH₂, CH₂CH₂, CH₂CH₂CH₂, CH₂(CH₂)₂CH₂, CH₂(CH₂)₃CH₂ and CH₂(CH₂)₄CH₂;
oxyalkylene: alkylene as defined above, preferably with 2 to 4 CH₂ groups, where one valency is attached to the skeleton via an oxygen atom. Examples of preferred oxyalkylene radicals are OCH₂, OCH₂CH₂, OCH₂CH₂CH₂ and OCH₂(CH₂)₂CH₂;
oxyalkyleneoxy: alkylene as defined above, preferably with 1 to 3 CH₂ groups, where both valencies are attached to the skeleton via an oxygen atom. Examples of preferred oxyalkyleneoxy radicals are OCH₂O, OCH₂CH₂O and OCH₂CH₂CH₂O;
alkylthio: alkyl as defined above which is attached via an S atom;
alkylsulfinyl: alkyl as defined above which is attached via an SO group;
alkylsulfonyl: alkyl as defined above which is attached via an S(O)₂ group;
five- or six-membered saturated, partially unsaturated or aromatic mono- or bicyclic heterocycle which contains one, two, three or four heteroatoms from the group consisting of O, N and S: the heterocycle in question may be attached via a carbon atom or via a nitrogen atom, if present. According to the invention it may be preferred for the heterocycle in question to be attached via carbon; on the other hand, it may also be preferred for the heterocycle to be attached via nitrogen. The heterocycle is in particular:
5- or 6-membered saturated or partially unsaturated heterocyclyl which contains one, two or three nitrogen atoms and/or one oxygen or sulfur atom or one or two oxygen and/or sulfur atoms, where the heterocyclyl may be attached via C or N;
5-membered heteroaryl which contains one, two, three or four nitrogen atoms or one, two or three nitrogen atoms and/or one sulfur- or oxygen atom which may be attached via C or N; or
6-membered heteroaryl which contains one, two, three or four, preferably one, two or three, nitrogen atoms which may be attached via C or N;
   5- or 6-membered saturated or partially unsaturated heterocyclyl, which contains one, two or three nitrogen atoms and/or one oxygen or sulfur atom or one or two oxygen and/or sulfur atoms, where the heterocyclyl may be attached via C or N, if present: for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazbiidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl;
   5-membered heteroaryl which contains one, two, three or four nitrogen atoms or one, two or three nitrogen atoms and/or one sulfur- or oxygen atom, where the heteroaryl may be attached via C or N, if present: 5-membered heteroaryl groups which, in addition to carbon atoms, may carry one to four nitrogen atoms or one to three nitrogen atoms and/or one sulfur or oxygen atom as ring members, for example furyl, thienyl, pyrrolyl, pyrazolyl, imidazolyl, triazolyl (1,2,3-; 1,2,4-triazolyl), tetrazolyl, oxazolyl, isoxazolyl, 1,3,4-oxadiazolyl, thiazolyl, isothiazolyl and thiadiazolyl, in particular 2-furyl,
   3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
   6-membered heteroaryl, which contains one, two, three or four, preferably one, two or three, nitrogen atoms, where the heteroaryl may be attached via C or N, if present: 6-membered heteroaryl groups which, in addition to carbon atoms, may contain one to four or one to three nitrogen atoms as ring members, for example pyridinyl, pyrimidinyl, pyrazinyl, pyridazinyl, 1,2,3-triazinyl, 1,2,4-triazinyl, 1,3,5-triazinyl, in particular 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

The scope of the present invention embraces the (R) and (S) isomers or rotamers and the racemates of compounds according to the invention having chiral centers. The compounds according to the invention may be present in various crystal modifications which may differ in their biological activity. They are likewise provided by the present invention.

With a view to the intended use of the pyrimidines according to formula I, particular preference is given to the following meanings of the substituents, in each case on their own or in combination. If appropriate, the preferred substituents or preferred combinations of substituents apply correspondingly to the precursors of the compounds according to the invention.

In a preferred embodiment of the invention, X is NR¹R². Here, it may be preferred if R² is hydrogen. In one embodiment of the present invention, R² is hydrogen and R¹ is different from hydrogen. Furthermore, it may be preferred for at least one of the radicals R¹ and R² to be different from hydrogen. Preference is likewise given to compounds of the formula I in which R¹ and R² are different from hydrogen. From among these, preference is given to compounds of the formula I in which R² is (C₁-C₄)-alkyl, (C₁-C₄)haloalkyl or (C₁-C₄)-alkoxy, especially methyl, ethyl or methoxy, in particular methyl or ethyl.

For preferred compounds of the formula I according to the invention, R¹ is straight-chain or branched unsubstituted or substituted (C₁-C₈)-alkyl, (C₁-C₈)-haloalkyl, (C₂-C₈)alkenyl, (C₂-C₈)-alkynyl, (C₃-C₈)-cycloalkyl, unsubstituted or substituted phenyl or naphthyl or an unsubstituted or substituted five- or six-membered saturated, partially unsaturated or aromatic heterocycle which contains one, two, three or four heteroatoms from the group consisting of O, N and S. R¹ is in particular (C₁-C₆)-alkyl, (C₂-C₆)-alkenyl, (C₂-C₆)-alkynyl, (C₃-C₆)-cycloalkyl, where these radicals may be substituted 1, 2, 3, 4 or 5 times by halogen, (C₁-C₆)-alkyl or (C₁-C₆)-haloalkyl. Furthermore preferably, R¹ is (C₃-C₆)-cycloalkyl which may be substituted by (C₁-C₄)-alkyl, or (C₃-C₆)-cycloalkyl(C₁-C₄)-alkyl. Also preferably, R¹ in the compounds of the formula I is selected from: methyl, ethyl, CH(CH₃)CH₂CH₃, CH₂CH(CH₃)₂, CH(CH₃)CH(CH₃)₂, CH(CH₃)C(CH₃)₃, CH(CH₃)CF₃, CH(CH₃)CF₃, CH(CH₃)CCl₃, CH₂CF₂CF₃, CH₂C(CH₃)═CH₂, CH₂CH═CH₂, cyclopentyl, cyclohexyl, cyclopropylmethyl, benzyl; and R² is preferably hydrogen or methyl.

Preference is also given to compounds of the formula I in which R¹ and R² together with the nitrogen atom to which they are attached are unsubstituted or substituted saturated or partially unsaturated heterocyclyl as defined above. From among these, preference is given to those compounds in which R¹ and R² together with the nitrogen atom to which they are attached form an unsubstituted or substituted 5- or 6-membered heterocycle, in particular a pyrrolidinyl, piperidinyl, morpholinyl, thiomorpholinyl or perhydro-(1,2)-oxazinyl ring, especially a pyrrolidinyl, piperidinyl or perhydro-(1,2)-oxazinyl ring. Here, heterocyclyl is in particular unsubstituted or substituted by 1, 2 or 3 substituents R^{a}, preferred substituents R^{a} on heterocyclyl being selected from the group consisting of halogen, (C₁-C₄)-alkyl and (C₁-C₄)-haloalkyl. Examples of preferred heterocyclyl rings are 2-methylpyrrolidinyl, 4-methylpiperidinyl, 4-trifluoromethylpiperidinyl, 3,4-dimethylpiperidinyl, 5-methylperhydro-(1,2)-oxazinyl and 6-methylperhydro-(1,2)oxazinyl.

In a further preferred embodiment, X is OR³. In yet a further preferred embodiment, X is SR³. Here, R³ is in each case preferably C₁-C₆-alkyl, in particular C₁-C₄-alkyl, C₁-C₆-haloalkyl, in particular C₁-C₄-haloalkyl, C₂-C₆-alkenyl, in particular C₂-C₄-alkenyl, C₂-C₆-alkynyl, in particular C₂-C₄-alkynyl, or C₃-C₆-cycloalkyl. With particular preference, R³ is C₁-C₆-alkyl, C₂-C₆-alkenyl or C₁-C₆-haloalkyl branched in the α-position. According to a particularly preferred embodiment of the invention, R³ is ethyl, propyl, isopropyl, 1,2-dimethylpropyl, 1,2,2-trimethylpropyl, 1-methyl-2,2,2-trifluoroethyl or 2,2,2-trifluoroethyl.

According to a further embodiment, X is C₁-C₈-alkyl which is optionally substituted by R^{a}.

According to a preferred embodiment, Y is (C₂-C₁₀)-alkenyl, preferably (C₂-C₆)-alkenyl. According to a further preferred embodiment, Y is (C₂-C₁₀)-alkynyl, preferably (C₂-C₆)alkynyl. Y may in each case carry one, two, three or four identical or different groups R^{u}, where the radicals R^{u} are as defined in the claims and are preferably each independently of the others selected from the group consisting of: halogen, cyano, C₁-C₄-alkyl, C₁-C₄-haloalkyl, C₁-C₄-alkoxy-C₁-C₄-alkyl, C₃-C₆-cycloalkyl, C₃-C₆-halocycloalkyl, C₁-C₄-alkoxy C₁-C₄-haloalkoxy and —Si(C₁-C₆-alkyl)₃.

According to a preferred embodiment, Y is (C₃-C₁₀)-cycloalkenyl, preferably (C₅-C₇)cycloalkenyl, group which is unsubstituted or substituted by R^{u}, as defined above. The cycloalkenyl group has preferably one or two double bonds. Examples of cycloalkenyl groups as substituent Y are cyclopent-1-en-1-yl, cyclohex-1-en-1-yl, which, according to the invention, may be substituted by R^{u}, as defined above.

According to a further preferred embodiment, Y is an unsubstituted or substituted five-, six-, seven-, eight-, nine- or ten-membered partially unsaturated heterocycle which contains one, two or three heteroatoms from the group consisting of O, N and S as ring members; here, Y is preferably an unsubstituted or substituted five- or six-membered partially unsaturated heterocycle which contains one, two or three nitrogen atoms and/or one oxygen or sulfur atom or one or two oxygen and/or sulfur atoms. In each case, the heterocyclyl ring may be attached via C or N to the pyrimidine skeleton. Furthermore preferably, the heterocycle contains one or two nitrogen atoms. Here, “partially unsaturated” means that the heterocycle does not form an aromatic system, where the heterocycle preferably contains one or two double bonds.

According to a preferred embodiment of the invention, Y is a group (A)

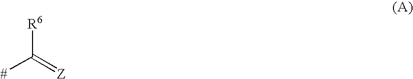

where # is the point of attachment to the pyrimidine skeleton. According to a preferred embodiment of the invention, Z is CR⁷R⁸, where R⁶, R⁷ and R⁸ are as defined in the claims and are preferably independently of one another: hydrogen, halogen, cyano, nitro, C₁-C₈-alkyl, C₁-C₈-haloalkyl, C₁-C₈-alkoxy-C₁-C₈-alkyl, C₂-C₈-alkenyl, C₂-C₈-haloalkenyl, C₂-C₈-alkynyl, C₂-C₈-haloalkynyl, C₄-C₁₀-alkadienyl, C₃-C₆-cycloalkyl, C₃-C₆-halocycloalkyl, C₄-C₆-cycloalkenyl, C₄-C₆-halocycloalkenyl, C₅-C₁₀-bicycloalkyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkoxy, C₃-C₈-halocycloalkoxy, C₃-C₈-cycloalkenyloxy, C₁-C₈-alkylcarbonyloxy, C₁-C₈-alkylaminothiocarbonyl, di-C₁-C₈-alkylaminothiocarbonyl, —C(═O)-A, —C(═O)—O-A, —C(═O)—N(A′)A, C(A′)(=N—OA), N(A′)A, N(A′)-C(═O)-A, N(A″)—C(═O)—N(A′)A, S(═O)ₘ-A, S(═O)ₘ—O-A, S(═O)ₘ—N(A′)A, —Si(C₁-C₆-alkyl)₃, phenyl, naphthyl, a five- to ten-membered saturated, partially unsaturated or aromatic heterocycle which contains one, two, three or four heteroatoms from the group consisting of O, N and S;

R⁶, R⁷ and/or R⁸ may carry one, two, three or four identical or different groups R^{a} where m, A, A′, A″ and R^{a} have the meanings and preferred meanings as defined further above.

R⁷ and R⁸ are preferably independently of one another selected from the group consisting of hydrogen, halogen, such as, for example, fluorine, chlorine and bromine; C₁-C₆-alkyl, in particular methyl, ethyl, n-propyl and iso-propyl; C₁-C₆-haloalkyl, in particular trichloromethyl, trifluoromethyl, 1,1,1-trifluorethyl; C₁-C₆-alkoxy, in particular methoxy, ethoxy and propoxy; or C₁-C₆-haloalkoxy, such as, for example, trichloromethoxy, trifluoromethoxy and 1,1,1-trifluoroethoxy. It is preferred for one of the substituents to be selected from the group consisting of hydrogen, halogen, such as fluorine, chlorine and bromine, and C₁-C₆-alkyl, in particular methyl, ethyl and isopropyl; and for the other substituent to be selected from the group consisting of halogen, such as fluorine, chlorine and bromine; C₁-C₆-alkyl, in particular methyl, ethyl; C₁-C₆-haloalkyl, in particular trichloromethyl, trifluoromethyl, 1,1,1-trifluoroethyl; and C₁-C₆-alkoxy, in particular methoxy, ethoxy and propoxy. However, it may be preferred that neither R⁷ nor R⁸ is hydrogen.

R⁶ is preferably hydrogen, halogen, C₁-C₈-alkoxy, C₁-C₈-alkyl, C₁-C₈-haloalkyl, C₁-C₈-alkoxy-C₁-C₈-alkyl, C₂-C₈-alkenyl, C₁-C₆-alkylamino or di-(C₁-C₆-alkyl)amino. Preferably, R⁶ is halogen, such as, for example fluorine, chlorine and bromine; C₁-C₆-alkyl, in particular methyl, ethyl, n-propyl and isopropyl; C₁-C₆-haloalkyl, in particular trichloromethyl, trifluoromethyl, 1,1,1-trifluoroethyl; C₁-C₆-alkoxy, in particular methoxy, ethoxy and propoxy; or C₁-C₆-haloalkoxy, such as, for example trichloromethoxy, trifluoromethoxy and 1,1,1-trifluoroethoxy. It may be preferred for R⁶ to be different from hydrogen.

According to a further preferred embodiment of the invention, Z is NR⁹. Here, R⁹ has the meanings as defined further above for R⁶, R⁷ and R⁸, where R⁶ and R⁹ may correspondingly form a cycle or a heterocycle and R⁶ and/or R⁹ or a cycle or heterocycle formed by R⁶ and R⁹ may be substituted correspondingly. Preferably, R⁹ is hydrogen, halogen, such as, for example, fluorine, chlorine and bromine; C₁-C₆-alkyl, in particular methyl, ethyl, n-propyl and isopropyl; C₁-C₆-haloalkyl, in particular trichloromethyl, trifluoromethyl, 1,1,1-trifluoroethyl; C₁-C₆-alkoxy, in particular methoxy, ethoxy and propoxy; or C₁-C₆-haloalkoxy, such as, for example trichloromethoxy, trifluoromethoxy and 1,1,1-trifluoroethoxy. R⁶ has the preferred meanings given above. It may be preferred for R⁶ and/or R⁹ to be different from hydrogen.

In a preferred embodiment of the present invention, R⁴ is a five- or six-membered aromatic heterocycle which contains one, two, three or four heteroatoms from the group consisting of O, N and S, where R⁴ may be partially or fully halogenated and/or may carry one, two, three or four identical or different groups R^{v}. Preferably, R⁴ is not pyridin-2-yl. According to a preferred embodiment thereof, R⁴ is a five-membered aromatic heterocycle, according to a further embodiment of the invention, it is a six-membered aromatic heterocycle; however, R⁴ is preferably not pyridin-2-yl. Examples of preferred five-membered aromatic heterocycles are pyrrolyl, pyrazolyl (in particular pyrazol-1-yl), imidazolyl, [1,2,3]-triazolyl (in particular [1,2,3]-triazol-1-yl), [1,2,4]-triazolyl (in particular[1,2,4]-triazol-1-yl), tetrazolyl, oxazolyl, isoxazolyl, [1,3,4]-oxadiazolyl, furyl, thienyl, thiazolyl and isothiazolyl, particularly preferably pyrazolyl, [1,2,3]-triazolyl and [1,2,4]triazolyl. Examples of preferred six-membered aromatic heterocycles are pyridin-3-yl, pyridin-4-yl, pyrimidinyl, pyrazinyl, pyridazinyl, [1,2,3]-triazinyl and [1,2,4]-triazinyl, preferably pyrimidinyl and pyridazinyl.

In a further preferred embodiment of the invention, R⁴ is an unsubstituted or substituted five- or six-membered saturated or partially unsaturated heterocycle. Furthermore preferably, R⁴ in the compounds of the formula I is 1-pyridin-(1,2-dihydro)-2-onyl, 1-pyrrolidone, imidazolidinone, isoxazolidinone or oxazolidinone, in particular 2-pyrrolidon-1-yl, imidazolidinon-1-yl, isoxazolidin-3-on-2-yl or oxazolidin-2-on-3-yl.

The heterocycle defined above may in each case be unsubstituted or substituted by one, two or three substituents R^{v} and, unless indicated otherwise, may in each case be attached via C or N to the pyrimidine skeleton. Both in combination with the broader definition of R^{v} given further above and in combination with the narrower definition of R^{v} below, this gives pyrimidines which are preferred according to the invention: halogen, cyano, C₁-C₈-alkyl, C₁-C₈-haloalkyl, C₁-C₆-alkoxy, C(═O)-A, C(═O)—O-A, C(═O)—N(A′)A, C(A′)(=N—OA), N(A′)A, N(A′)-C(═O)-A, in particular halogen, cyano, C₁-C₈-alkyl or C₁-C₈-haloalkyl.

In a further preferred embodiment of the invention, R⁴ is

cyano, C(═W)OR^{c}, C(═W)NR^{z}R^{d}, C(═W)NR^{c}—NR^{z}R^{d}, C(═W)R^{c}, CR^{c}R^{d}—OR^{z}, CR^{c}R^{d}—NR^{z}R^{f}, ON(═CR^{c}R^{d}), O—C(═W)R^{c},
NR^{c}R^{d}, NR^{c}(C(═W)R^{d}), NR^{c}(C(═W)OR^{d}), NR^{c}(C(═W)—NR^{z}R^{d}),
NR^{c}(N═CR^{f}R^{d}), NR^{c}NR^{z}R^{d}, NR^{z}—OR^{c}, where the substituents are preferably as defined below:
W is O, NR^{g} or NOR^{g}
R^{c}, R^{d}, R^{f}, R^{g} independently of one another are hydrogen, C₁-C₆-alkyl, C₂-C₆-alkenyl, C₂-C₆-alkynyl, C₃-C₆-cycloalkyl or C₄-C₆-cycloalkenyl;
R^{y}, R^{z} independently have the same meanings as R^{c} and may additionally be C(═O)—R^{g} or C(═O)—OR^{g};
   where the aliphatic and/or alicyclic groups of the radical definitions of R^{c}, R^{d}, R^{f}, R^{g}, R^{y} and R^{z} for their part may be partially or fully halogenated and/or may carry one, two, three or four identical or different groups R^{w}:
R^{w} is halogen, cyano, C₁-C₈-alkyl, C₂-C₁₀-alkenyl, C₃-C₆-cycloalkyl, C₃-C₆-cycloalkenyl, C₁-C₆-alkoxy;

Here, it may be preferred if the substituent R^{d} in NR^{c}R^{d} is not hydrogen.

According to a further preferred embodiment, R⁴ is cyano, C(═W)OR^{c}, C(═W)NR^{z}R^{d}, C(═W)NR^{c}—NR^{z}R^{d}, C(═W)R^{c}, CR^{c}R^{d}—OR^{z}, CR^{c}R^{d}—NR^{z}R^{f}, where the substituents have the meanings and preferred meanings as defined further above. In a preferred embodiment, R⁴ is C(═O)NR^{z}R^{d}, C(═NOR⁹)NR^{z}R^{d}, C(═NOR^{g})R^{c}, C(═N—NR^{z}R^{d})R^{c} or CR^{c}R^{d}—NR^{z}R^{f}. Preference is furthermore given to C(═W)OR^{c}, C(═W)NR^{z}R^{d} or C(═W)R^{c}, where W=O or NOR^{g}, R^{c}, R^{d}, R^{g}, R^{z} independently of one another are hydrogen or C₁-C₆-alkyl and R^{z} may additionally be C(═O)—R^{g} or C(═O)—OR^{g}. Specific examples of such preferred radicals R⁴ are C(═O)NH₂ or C(═N—OCH₃)NH₂.

Preference is furthermore given to compounds acording to the invention in which R⁴ is C(═NH)NR^{z}R^{d} and R^{z} is a substituent C(═O)R^{g} or C(═O)—OR⁹.

In a further preferred embodiment, R⁴ is NR^{c}C(═W)R^{d}), NR^{c}C(═W)OR^{d}), NR^{c}C(═W)—NR^{z}R^{d}) or NR^{z}—OR^{c}, where the substituents have the meanings and preferred meanings as defined further above.

In preferred pyrimidines I according to the invention, R⁵ is halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₆-halocycloalkyl, C₁-C₆-alkoxy, C₁-C₆-haloalkoxy, C₂-C₆-alkenyloxy, C₃-C₄-alkynyloxy, C₁-C₈-alkylthio, C₁-C₆-alkylamino, di-(C₁-C₆-alkyl)amine, where the aliphatic and alicyclic groups of the radical definitions of R⁵ for their part may contain one, two, three or four substituents independently of one another selected from the group consisting of halogen, cyano, nitro, C₁-C₂-alkoxy and C₁-C₂-alkoxycarbonyl.

In further preferred compounds according to the invention, R⁵ is halogen, cyano, C₁-C₄-alkoxy or C₁-C₄-haloalkoxy, in particular halogen. Specific examples of such suitable radicals R⁵ are chlorine, cyano, methoxy and trifluoromethoxy. In a further preferred embodiment, R⁵ is C₁-C₄-alkyl or C₁-C₄-haloalkyl, in particular methyl, ethyl or trifluoromethyl.

The following substructure are preferred embodiments of the compounds of the formula I, where the substituents present therein each have the meanings and preferred meanings as described for formula I.

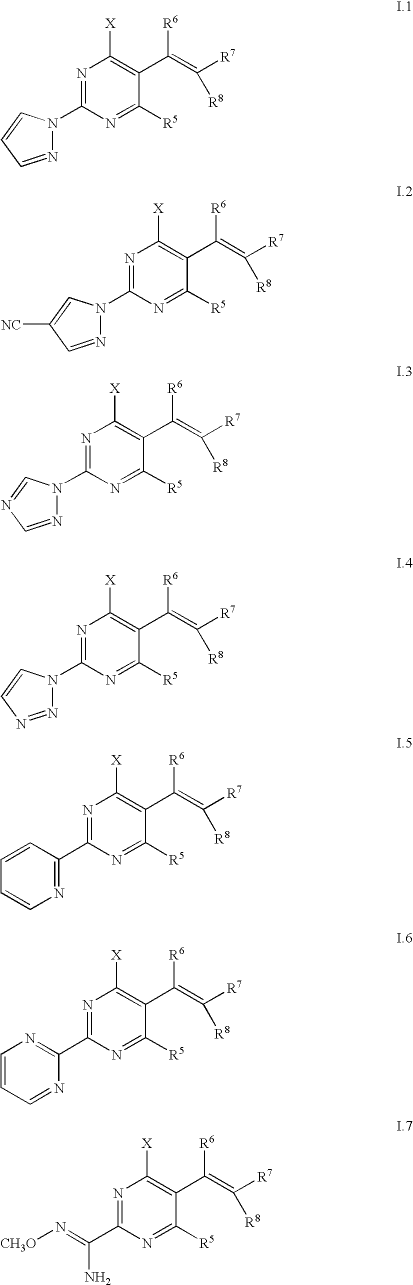

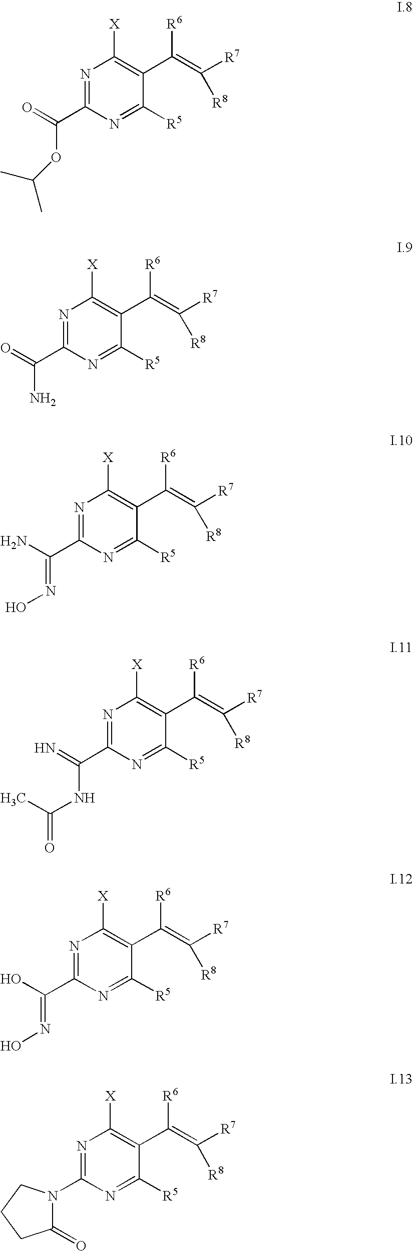

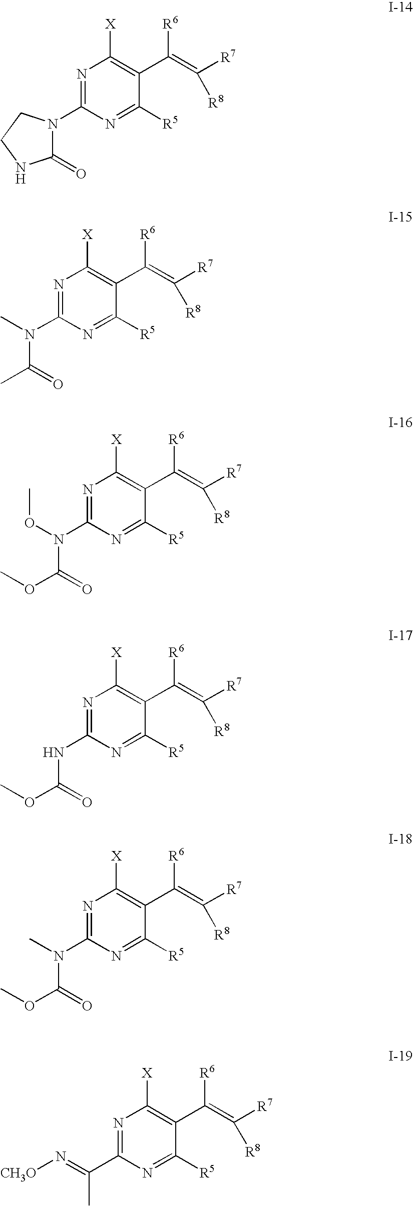

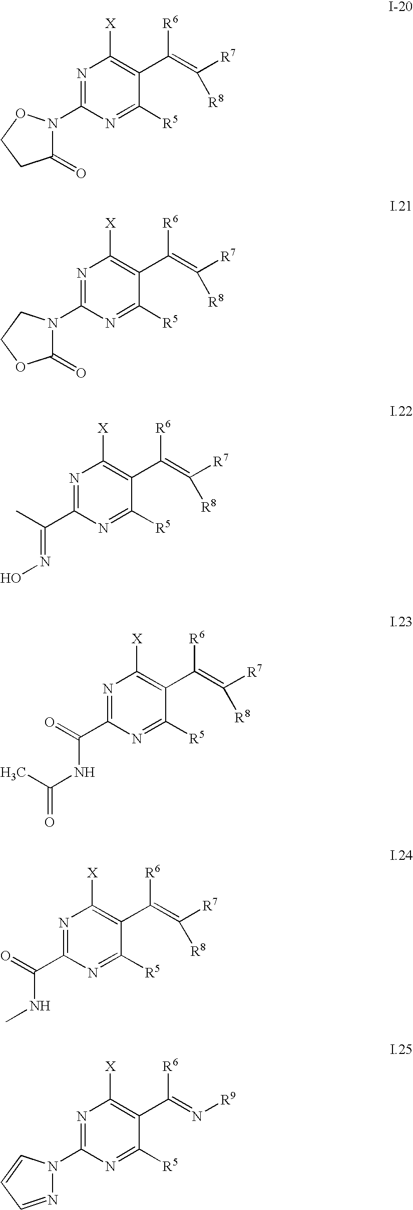

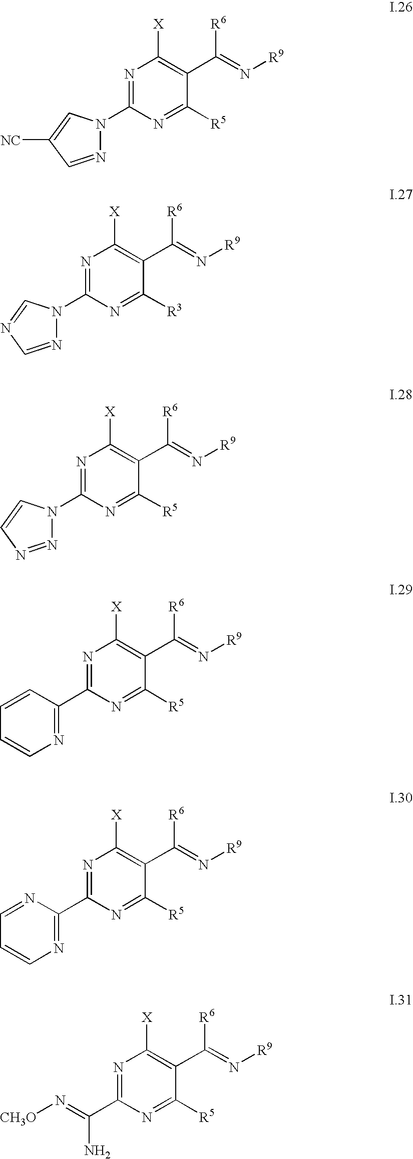

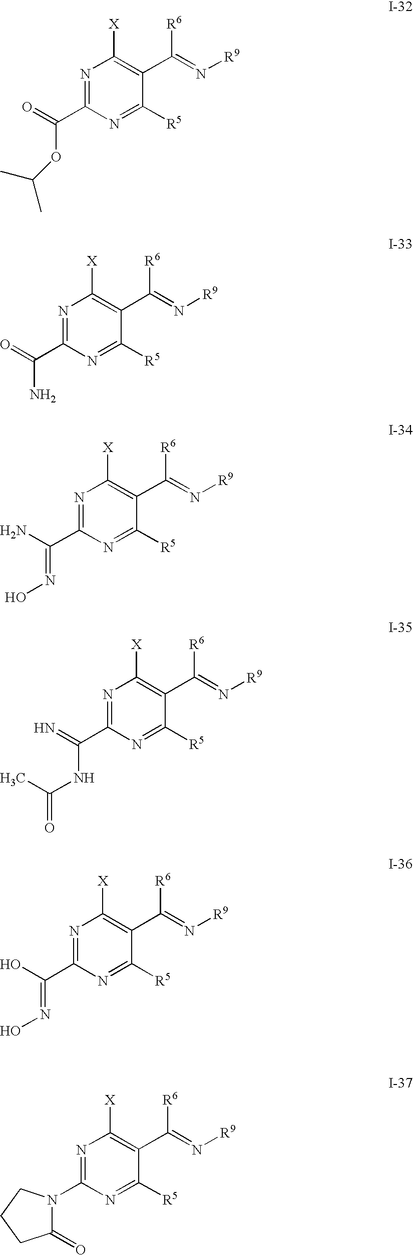

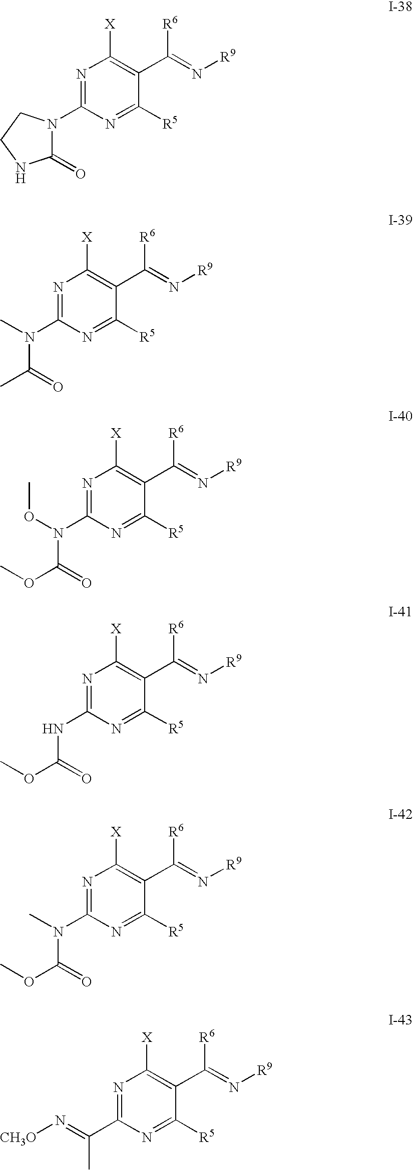

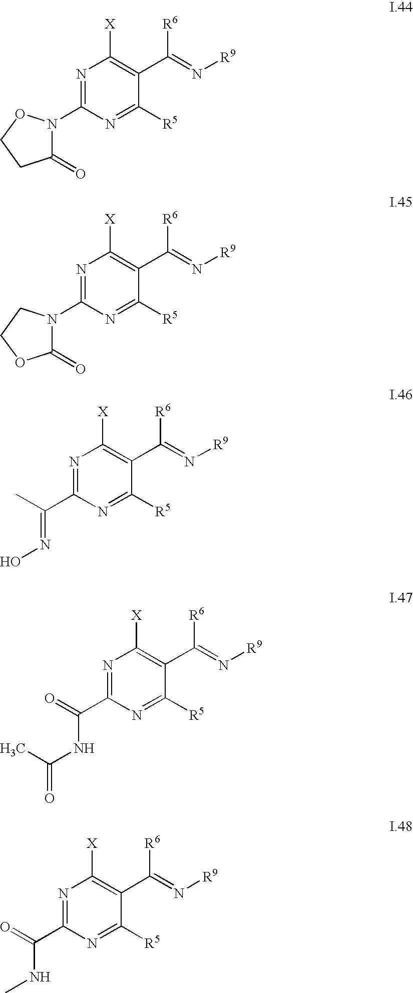

In accordance with the present invention, particularly preferred compounds are the compounds I compiled in the tables below. The groups mentioned in the tables for a substituent are furthermore per se, independently of the combination in which they are mentioned, a particularly preferred embodiment of the substituent in question.

### Table 1

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶, R⁷, R⁸ are each chlorine, X is NR¹R² and R¹ and R² for a particular compound correspond in each case to one row of Table A

### Table 2

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is chlorine, R⁸ is bromine, X is NR¹R² and R¹ and R² for a particular compound correspond in each case to one row of Table A

### Table 3

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is chlorine, R⁸ is methyl, X is NR¹R² and R¹ and R² for a particular compound correspond in each case to one row of Table A

### Table 4

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is chlorine, R⁸ is methoxy, X is NR¹R² and R¹ and R² for a particular compound correspond in each case to one row of Table A

### Table 5

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is bromine, R⁸ is chlorine, X is NR¹R² and R¹ and R² for a particular compound correspond in each case to one row of Table A

### Table 6

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is bromine, R⁸ is bromine, X is NR¹R² and R¹ and R² for a particular compound correspond in each case to one row of Table A

### Table 7

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is bromine, R⁸ is methyl, X is NR¹R² and R¹ and R² for a particular compound correspond in each case to one row of Table A

### Table 8

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is bromine, R⁸ is methoxy, X is NR¹R² and R¹ and R² for a particular compound correspond in each case to one row of Table A

### Table 9

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is methyl, R⁸ is chlorine, X is NR¹R² and R¹ and R² for a particular compound correspond in each case to one row of Table A

### Table 10

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is methyl, R⁸ is bromine, X is NR¹R² and R¹ and R² for a particular compound correspond in each case to one row of Table A

### Table 11

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is methyl, R⁸ is methyl, X is NR¹R² and R¹ and R² for a particular compound correspond in each case to one row of Table A

### Table 12

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is methyl, R⁸ is methoxy, X is NR¹R² and R¹ and R² for a particular compound correspond in each case to one row of Table A

### Table 13

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is methoxy, R¹ is chlorine, X is NR¹R² and R¹ and R² for a particular compound correspond in each case to one row of Table A

### Table 14

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is methoxy, R⁸ is bromine, X is NR¹R² and R¹ and R² for a particular compound correspond in each case to one row of Table A

### Table 15

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is methoxy, R⁸ is methyl, X is NR¹R² and R¹ and R² for a particular compound correspond in each case to one row of Table A

### Table 16

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is methoxy, R⁸ is methoxy, X is NR¹R² and R¹ and R² for a particular compound correspond in each case to one row of Table A

### Table 17

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is chlorine, R⁸ is chlorine, X is NR¹R² and R¹ and R² for a particular compound correspond in each case to one row of Table A

### Table 18

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is chlorine, R⁸ is bromine, X is NR¹R² and R¹ and R² for a particular compound correspond in each case to one row of Table A

### Table 19

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is chlorine, R⁸ is methyl, X is NR¹R² and R¹ and R² for a particular compound correspond in each case to one row of Table A

### Table 20

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is chlorine, R⁸ is methoxy, X is NR¹R² and R¹ and R² for a particular compound correspond in each case to one row of Table A

### Table 21

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is bromine, R⁸ is chlorine, X is NR¹R² and R¹ and R² for a particular compound correspond in each case to one row of Table A

### Table 22

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶, R⁷, R⁸ are each bromine, X is NR¹R² and R¹ and R² for a particular compound correspond in each case to one row of Table A

### Table 23

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is bromine, R⁸ is methyl, X is NR¹R² and R¹ and R² for a particular compound correspond in each case to one row of Table A

### Table 24

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is bromine, R⁸ is methoxy, X is NR¹R² and R¹ and R² for a particular compound correspond in each case to one row of table A

### Table 25

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is methyl, R⁸ is chlorine, X is NR¹R² and R¹ and R² for a particular compound correspond in each case to one row of Table A

### Table 26

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is methyl, R⁸ is bromine, X is NR¹R² and R¹ and R² for a particular compound correspond in each case to one row of Table A

### Table 27

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is methyl, R⁸ is methyl, X is NR¹R² and R¹ and R² for a particular compound correspond in each case to one row of Table A

### Table 28

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is methyl, R⁸ is methoxy, X is NR¹R² and R¹ and R² for a particular compound correspond in each case to one row of Table A

### Table 29

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is methoxy, R⁸ is chlorine, X is NR¹R² and R¹ and R² for a particular compound correspond in each case to one row of Table A

### Table 30

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is methoxy, R⁸ is bromine, X is NR¹R² and R¹ and R² for a particular compound correspond in each case to one row of Table A

### Table 31

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is methoxy, R⁸ is methyl, X is NR¹R² and R¹ and R² for a particular compound correspond in each case to one row of Table A

### Table 32

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is methoxy, R⁸ is methoxy, X is NR¹R² and R¹ and R² for a particular compound correspond in each case to one row of Table A

### Table 33

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is chlorine, R⁸ is chlorine, X is NR¹R² and R¹ and R² for a particular compound correspond in each case to one row of Table A

### Table 34

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is chlorine, R⁸ is bromine, X is NR¹R² and R¹ and R² for a particular compound correspond in each case to one row of Table A

### Table 35

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is chlorine, R⁸ is methyl, X is NR¹R² and R¹ and R² for a particular compound correspond in each case to one row of Table A

### Table 36

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is chlorine, R⁸ is methoxy, X is NR¹R² and R¹ and R² for a particular compound correspond in each case to one row of Table A

### Table 37

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is bromine, R⁸ is chlorine, X is NR¹R² and R¹ and R² for a particular compound correspond in each case to one row of Table A

### Table 38

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is bromine, R⁸ is bromine, X is NR¹R² and R¹ and R² for a particular compound correspond in each case to one row of Table A

### Table 39

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is bromine, R⁸ is methyl, X is NR¹R² and R¹ and R² for a particular compound correspond in each case to one row of Table A

### Table 40

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is bromine, R⁸ is methoxy, X is NR¹R² and R¹ and R² for a particular compound correspond in each case to one row of Table A

### Table 41

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is methyl, R⁸ is chlorine, X is NR¹R² and R¹ and R² for a particular compound correspond in each case to one row of Table A

### Table 42

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is methyl, R⁸ is bromine, X is NR¹R² and R¹ and R² for a particular compound correspond in each case to one row of Table A

### Table 43

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶, R⁷, R⁸ are each methyl, X is NR¹R² and R¹ and R² for a particular compound correspond in each case to one row of Table A

### Table 44

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is methyl, R⁸ is methoxy, X is NR¹R² and R¹ and R² for a particular compound correspond in each case to one row of Table A

### Table 45

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is methoxy, R⁸ is chlorine, X is NR¹R² and R¹ and R² for a particular compound correspond in each case to one row of Table A

### Table 46

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is methoxy, R⁸ is bromine, X is NR¹R² and R¹ and R² for a particular compound correspond in each case to one row of Table A

### Table 47

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is methoxy, R⁸ is methyl, X is NR¹R² and R¹ and R² for a particular compound correspond in each case to one row of Table A

### Table 48

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is methoxy, R⁸ is methoxy, X is NR¹R² and R¹ and R² for a particular compound correspond in each case to one row of Table A

### Table 49

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is chlorine, R⁸ is chlorine, X is NR¹R² and R¹ and R² for a particular compound correspond in each case to one row of Table A

### Table 50

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is chlorine, R⁸ is bromine, X is NR¹R² and R¹ and R² for a particular compound correspond in each case to one row of Table A

### Table 51

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is chlorine, R⁸ is methyl, X is NR¹R² and R¹ and R² for a particular compound correspond in each case to one row of Table A

### Table 52

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is chlorine, R⁸ is methoxy, X is NR¹R² and R¹ and R² for a particular compound correspond in each case to one row of Table A

### Table 53

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is bromine, R⁸ is chlorine, X is NR¹R² and R¹ and R² for a particular compound correspond in each case to one row of Table A

### Table 54

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is bromine, R⁸ is bromine, X is NR¹R² and R¹ and R² for a particular compound correspond in each case to one row of Table A

### Table 55

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is bromine, R⁸ is methyl, X is NR¹R² and R¹ and R² for a particular compound correspond in each case to one row of Table A

### Table 56

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is bromine, R⁸ is methoxy, X is NR¹R² and R¹ and R² for a particular compound correspond in each case to one row of Table A

### Table 57

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is methyl, R⁸ is chlorine, X is NR¹R² and R¹ and R² for a particular compound correspond in each case to one row of Table A

### Table 58

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is methyl, R⁸ is bromine, X is NR¹R² and R¹ and R² for a particular compound correspond in each case to one row of Table A

### Table 59

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is methyl, R⁸ is methyl, X is NR¹R² and R¹ and R² for a particular compound correspond in each case to one row of Table A

### Table 60

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is methyl, R⁸ is methoxy, X is NR¹R² and R¹ and R² for a particular compound correspond in each case to one row of Table A

### Table 61

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is methoxy, R⁸ is chlorine, X is NR¹R² and R¹ and R² for a particular compound correspond in each case to one row of Table A

### Table 62

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is methoxy, R⁸ is bromine, X is NR¹R² and R¹ and R² for a particular compound correspond in each case to one row of Table A

### Table 63

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is methoxy, R⁸ is methyl, X is NR¹R² and R¹ and R² for a particular compound correspond in each case to one row of Table A

### Table 64

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶, R⁷, R⁸ are each methoxy, X is NR¹R² and R¹ and R² for a particular compound correspond in each case to one row of Table A

### Table 65

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is chlorine, R⁶ is chlorine, R⁹ is methoxy, X is NR¹R² and R¹ and R² for a particular compound correspond in each case to one row of Table A

### Table 66

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is chlorine, R⁶ is bromine, R⁹ is methoxy, X is NR¹R² and R¹ and R² for a particular compound correspond in each case to one row of Table A

### Table 67

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is chlorine, R⁶ is methyl, R⁹ is methoxy, X is NR¹R² and R¹ and R² for a particular compound correspond in each case to one row of Table A

### Table 68

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is chlorine, R⁶ is chlorine, R⁹ is methyl, X is NR¹R² and R¹ and R² for a particular compound correspond in each case to one row of Table A

### Table 69

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is chlorine, R⁶ is bromine, R⁹ is methyl, X is NR¹R² and R¹ and R² for a particular compound correspond in each case to one row of Table A

### Table 70

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is chlorine, R⁶ is methyl, R⁹ is methyl, X is NR¹R² and R¹ and R² for a particular compound correspond in each case to one row of Table A

### Table 71

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is chlorine, R⁶ is chlorine, R⁹ is chlorine, X is NR¹R² and R¹ and R² for a particular compound correspond in each case to one row of Table A

### Table 72

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is chlorine, R⁶ is bromine, R⁹ is chlorine, X is NR¹R² and R¹ and R² for a particular compound correspond in each case to one row of Table A

### Table 73

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is chlorine, R⁶ is methyl, R⁹ is chlorine, X is NR¹R² and R¹ and R² for a particular compound correspond in each case to one row of Table A

### Table 74

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶, R⁷, R⁸ are each chlorine and X for a particular compound corresponds in each case to one row of Table B

### Table 75

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is chlorine, R⁸ is bromine and X for a particular compound corresponds in each case to one row of Table B

### Table 76

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is chlorine, R⁸ is methyl and X for a particular compound corresponds in each case to one row of Table B

### Table 77

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is chlorine, R⁸ is methoxy and X for a particular compound corresponds in each case to one row of Table B

### Table 78

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is bromine, R⁸ is chlorine and X for a particular compound corresponds in each case to one row of Table B

### Table 79

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is bromine, R⁸ is bromine and X for a particular compound corresponds in each case to one row of Table B

### Table 80

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is bromine, R⁸ is methyl and X for a particular compound corresponds in each case to one row of Table B

### Table 81

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is bromine, R⁸ is methoxy and X for a particular compound corresponds in each case to one row of Table B

### Table 82

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is methyl, R⁸ is chlorine and X for a particular compound corresponds in each case to one row of Table B

### Table 83

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is methyl, R⁸ is bromine and X for a particular compound corresponds in each case to one row of Table B

### Table 84

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is methyl, R⁸ is methyl and X for a particular compound corresponds in each case to one row of Table B

### Table 85

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is methyl, R⁸ is methoxy and X for a particular compound corresponds in each case to one row of Table B

### Table 86

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is methoxy, R⁸ is chlorine and X for a particular compound corresponds in each case to one row of Table B

### Table 87

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is methoxy, R⁸ is bromine and X for a particular compound corresponds in each case to one row of Table B

### Table 88

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is methoxy, R⁸ is methyl and X for a particular compound corresponds in each case to one row of Table B

### Table 89

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is chlorine, R⁷ is methoxy, R⁸ is methoxy and X for a particular compound corresponds in each case to one row of Table B

### Table 90

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is chlorine, R⁸ is chlorine and X for a particular compound corresponds in each case to one row of Table B

### Table 91

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is chlorine, R⁸ is bromine and X for a particular compound corresponds in each case to one row of Table B

### Table 92

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is chlorine, R⁸ is methyl and X for a particular compound corresponds in each case to one row of Table B

### Table 93

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is chlorine, R⁸ is methoxy and X for a particular compound corresponds in each case to one row of Table B

### Table 94

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is bromine, R⁸ is chlorine and X for a particular compound corresponds in each case to one row of Table B

### Table 95

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶, R⁷, R⁸ are each bromine and X for a particular compound corresponds in each case to one row of Table B

### Table 96

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is bromine, R⁸ is methyl and X for a particular compound corresponds in each case to one row of Table B

### Table 97

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is bromine, R⁸ is methoxy and X for a particular compound corresponds in each case to one row of Table B

### Table 98

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is methyl, R⁸ is chlorine and X for a particular compound corresponds in each case to one row of Table B

### Table 99

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is methyl, R⁸ is bromine and X for a particular compound corresponds in each case to one row of Table B

### Table 100

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is methyl, R⁸ is methyl and X for a particular compound corresponds in each case to one row of Table B

### Table 101

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is methyl, R⁸ is methoxy and X for a particular compound corresponds in each case to one row of Table B

### Table 102

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is methoxy, R⁸ is chlorine and X for a particular compound corresponds in each case to one row of Table B

### Table 103

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is methoxy, R⁸ is bromine and X for a particular compound corresponds in each case to one row of Table B

### Table 104

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is methoxy, R⁸ is methyl and X for a particular compound corresponds in each case to one row of Table B

### Table 105

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is bromine, R⁷ is methoxy, R⁸ is methoxy and X for a particular compound corresponds in each case to one row of Table B

### Table 106

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is chlorine, R⁸ is chlorine and X for a particular compound corresponds in each case to one row of Table B

### Table 107

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is chlorine, R⁸ is bromine and X for a particular compound corresponds in each case to one row of Table B

### Table 108

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is chlorine, R⁸ is methyl and X for a particular compound corresponds in each case to one row of Table B

### Table 109

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is chlorine, R⁸ is methoxy and X for a particular compound corresponds in each case to one row of Table B

### Table 110

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is bromine, R⁸ is chlorine and X for a particular compound corresponds in each case to one row of Table B

### Table 111

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is bromine, R⁸ is bromine and X for a particular compound corresponds in each case to one row of Table B

### Table 112

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is bromine, R⁸ is methyl and X for a particular compound corresponds in each case to one row of Table B

### Table 113

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is bromine, R⁸ is methoxy and X for a particular compound corresponds in each case to one row of Table B

### Table 114

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is methyl, R⁸ is chlorine and X for a particular compound corresponds in each case to one row of Table B

### Table 115

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is methyl, R⁸ is bromine and X for a particular compound corresponds in each case to one row of Table B

### Table 116

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶, R⁷, R⁸ are each methyl and X for a particular compound corresponds in each case to one row of Table B

### Table 117

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is methyl, R⁸ is methoxy and X for a particular compound corresponds in each case to one row of Table B

### Table 118

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is methoxy, R⁸ is chlorine and X for a particular compound corresponds in each case to one row of Table B

### Table 119

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is methoxy, R⁸ is bromine and X for a particular compound corresponds in each case to one row of Table B

### Table 120

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is methoxy, R⁸ is methyl and X for a particular compound corresponds in each case to one row of Table B

### Table 121

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methyl, R⁷ is methoxy, R⁸ is methoxy and X for a particular compound corresponds in each case to one row of Table B

### Table 122

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is chlorine, R⁸ is chlorine and X for a particular compound corresponds in each case to one row of Table B

### Table 123

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is chlorine, R⁸ is bromine and X for a particular compound corresponds in each case to one row of Table B

### Table 124

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is chlorine, R⁸ is methyl and X for a particular compound corresponds in each case to one row of Table B

### Table 125

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is chlorine, R⁸ is methoxy and X for a particular compound corresponds in each case to one row of Table B

### Table 126

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is bromine, R⁸ is chlorine and X for a particular compound corresponds in each case to one row of Table B

### Table 127

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is bromine, R⁸ is bromine and X for a particular compound corresponds in each case to one row of Table B

### Table 128

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is bromine, R⁸ is methyl and X for a particular compound corresponds in each case to one row of Table B

### Table 129

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is bromine, R⁸ is methoxy and X for a particular compound corresponds in each case to one row of Table B

### Table 130

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is methyl, R⁸ is chlorine and X for a particular compound corresponds in each case to one row of Table B

### Table 131

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is methyl, R⁸ is bromine and X for a particular compound corresponds in each case to one row of Table B

### Table 132

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is methyl, R⁸ is methyl and X for a particular compound corresponds in each case to one row of Table B

### Table 133

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is methyl, R⁸ is methoxy and X for a particular compound corresponds in each case to one row of Table B

### Table 134

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is methoxy, R⁸ is chlorine and X for a particular compound corresponds in each case to one row of Table B

### Table 135

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is methoxy, R⁸ is bromine and X for a particular compound corresponds in each case to one row of Table B

### Table 136

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶ is methoxy, R⁷ is methoxy, R⁸ is methyl and X for a particular compound corresponds in each case to one row of Table B

### Table 137

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is chlorine, R⁶, R⁷, R⁸ are each methoxy and X for a particular compound corresponds in each case to one row of Table B

### Table 138

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is chlorine, R⁶ is chlorine, R⁹ is methoxy and X for a particular compound corresponds in each case to one row of Table B

### Table 139

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is chlorine, R⁶ is bromine, R⁹ is methoxy and X for a particular compound corresponds in each case to one row of Table B

### Table 140

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is chlorine, R⁶ is methyl, R⁹ is methoxy and X for a particular compound corresponds in each case to one row of Table B

### Table 141

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is chlorine, R⁶ is chlorine, R⁹ is methyl and X for a particular compound corresponds in each case to one row of Table B

### Table 142

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is chlorine, R⁶ is bromine, R⁹ is methyl and X for a particular compound corresponds in each case to one row of Table B

### Table 143

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is chlorine, R⁶ is methyl, R⁹ is methyl and X for a particular compound corresponds in each case to one row of Table B

### Table 144

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is chlorine, R⁶ is chlorine, R⁹ is chlorine and X for a particular compound corresponds in each case to one row of Table B

### Table 145

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is chlorine, R⁶ is bromine, R⁹ is chlorine and X for a particular compound corresponds in each case to one row of Table B

### Table 146

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is chlorine, R⁶ is methyl, R⁹ is chlorine and X for a particular compound corresponds in each case to one row of Table B

### Table 147

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶, R⁷, R⁸ are each chlorine, X is NR¹R² and R¹ and R² for a particular compound correspond in each case to one row of Table A

### Table 148

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is chlorine, R⁸ is bromine, X is NR¹R² and R¹ and R² for a particular compound correspond in each case to one row of Table A

### Table 149

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is chlorine, R⁸ is methyl, X is NR¹R² and R¹ and R² for a particular compound correspond in each case to one row of Table A

### Table 150

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is chlorine, R⁸ is methoxy, X is NR¹R² and R¹ and R² for a particular compound correspond in each case to one row of Table A

### Table 151

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is bromine, R⁸ is chlorine, X is NR¹R² and R¹ and R² for a particular compound correspond in each case to one row of Table A

### Table 152

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is bromine, R⁸ is bromine, X is NR¹R² and R¹ and R² for a particular compound correspond in each case to one row of Table A

### Table 153

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is bromine, R⁸ is methyl, X is NR¹R² and R¹ and R² for a particular compound correspond in each case to one row of Table A

### Table 154

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is bromine, R⁸ is methoxy, X is NR¹R² and R¹ and R² for a particular compound correspond in each case to one row of Table A

### Table 155

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is methyl, R⁸ is chlorine, X is NR¹R² and R¹ and R² for a particular compound correspond in each case to one row of Table A

### Table 156

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is methyl, R⁸ is bromine, X is NR¹R² and R¹ and R² for a particular compound correspond in each case to one row of Table A

### Table 157

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is methyl, R⁸ is methyl, X is NR¹R² and R¹ and R² for a particular compound correspond in each case to one row of Table A

### Table 158

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is methyl, R⁸ is methoxy, X is NR¹R² and R¹ and R² for a particular compound correspond in each case to one row of Table A

### Table 159

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is methoxy, R⁸ is chlorine, X is NR¹R² and R¹ and R² for a particular compound correspond in each case to one row of Table A

### Table 160

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is methoxy, R⁸ is bromine, X is NR¹R² and R¹ and R² for a particular compound correspond in each case to one row of Table A

### Table 161

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is methoxy, R⁸ is methyl, X is NR¹R² and R¹ and R² for a particular compound correspond in each case to one row of Table A

### Table 162

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is methoxy, R⁸ is methoxy, X is NR¹R² and R¹ and R² for a particular compound correspond in each case to one row of Table A

### Table 163

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is chlorine, R⁸ is chlorine, X is NR¹R² and R¹ and R² for a particular compound correspond in each case to one row of Table A

### Table 164

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is chlorine, R⁸ is bromine, X is NR¹R² and R¹ and R² for a particular compound correspond in each case to one row of Table A

### Table 165

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is chlorine, R⁸ is methyl, X is NR¹R² and R¹ and R² for a particular compound correspond in each case to one row of Table A

### Table 166

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is chlorine, R⁸ is methoxy, X is NR¹R² and R¹ and R² for a particular compound correspond in each case to one row of Table A

### Table 167

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is bromine, R⁸ is chlorine, X is NR¹R² and R¹ and R² for a particular compound correspond in each case to one row of Table A

### Table 168

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶, R⁷, R⁸ are each bromine, X is NR¹R² and R¹ and R² for a particular compound correspond in each case to one row of Table A

### Table 169

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is bromine, R⁸ is methyl, X is NR¹R² and R¹ and R² for a particular compound correspond in each case to one row of Table A

### Table 170

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is bromine, R⁸ is methoxy, X is NR¹R² and R¹ and R² for a particular compound correspond in each case to one row of Table A

### Table 171

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is methyl, R⁸ is chlorine, X is NR¹R² and R¹ and R² for a particular compound correspond in each case to one row of Table A

### Table 172

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is methyl, R⁸ is bromine, X is NR¹R² and R¹ and R² for a particular compound correspond in each case to one row of Table A

### Table 173

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is methyl, R⁸ is methyl, X is NR¹R² and R¹ and R² for a particular compound correspond in each case to one row of Table A

### Table 174

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is methyl, R⁸ is methoxy, X is NR¹R² and R¹ and R² for a particular compound correspond in each case to one row of Table A

### Table 175

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is methoxy, R⁸ is chlorine, X is NR¹R² and R¹ and R² for a particular compound correspond in each case to one row of Table A

### Table 176

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is methoxy, R⁸ is bromine, X is NR¹R² and R¹ and R² for a particular compound correspond in each case to one row of Table A

### Table 177

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is methoxy, R⁸ is methyl, X is NR¹R² and R¹ and R² for a particular compound correspond in each case to one row of Table A

### Table 178

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is methoxy, R⁸ is methoxy, X is NR¹R² and R¹ and R² for a particular compound correspond in each case to one row of Table A

### Table 179

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is chlorine, R⁸ is chlorine, X is NR¹R² and R¹ and R² for a particular compound correspond in each case to one row of Table A

### Table 180

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is chlorine, R⁸ is bromine, X is NR¹R² and R¹ and R² for a particular compound correspond in each case to one row of Table A

### Table 181

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is chlorine, R⁸ is methyl, X is NR¹R² and R¹ and R² for a particular compound correspond in each case to one row of Table A

### Table 182

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is chlorine, R⁸ is methoxy, X is NR¹R² and R¹ and R² for a particular compound correspond in each case to one row of Table A

### Table 183

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is bromine, R⁸ is chlorine, X is NR¹R² and R¹ and R² for a particular compound correspond in each case to one row of Table A

### Table 184

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is bromine, R⁸ is bromine, X is NR¹R² and R¹ and R² for a particular compound correspond in each case to one row of Table A

### Table 185

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is bromine, R⁸ is methyl, X is NR¹R² and R¹ and R² for a particular compound correspond in each case to one row of Table A

### Table 186

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is bromine, R⁸ is methoxy, X is NR¹R² and R¹ and R² for a particular compound correspond in each case to one row of Table A

### Table 187

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is methyl, R⁸ is chlorine, X is NR¹R² and R¹ and R² for a particular compound correspond in each case to one row of Table A

### Table 188

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is methyl, R⁸ is bromine, X is NR¹R² and R¹ and R² for a particular compound correspond in each case to one row of Table A

### Table 189

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶, R⁷, R⁸ are each methyl, X is NR¹R² and R¹ and R² for a particular compound correspond in each case to one row of Table A

### Table 190

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is methyl, R⁸ is methoxy, X is NR¹R² and R¹ and R² for a particular compound correspond in each case to one row of Table A

### Table 191

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is methoxy, R⁸ is chlorine, X is NR¹R² and R¹ and R² for a particular compound correspond in each case to one row of Table A

### Table 192

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is methoxy, R⁸ is bromine, X is NR¹R² and R¹ and R² for a particular compound correspond in each case to one row of Table A

### Table 193

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is methoxy, R⁸ is methyl, X is NR¹R² and R¹ and R² for a particular compound correspond in each case to one row of Table A

### Table 194

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is methoxy, R⁸ is methoxy, X is NR¹R² and R¹ and R² for a particular compound correspond in each case to one row of Table A

### Table 195

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is chlorine, R⁸ is chlorine, X is NR¹R² and R¹ and R² for a particular compound correspond in each case to one row of Table A

### Table 196

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is chlorine, R⁸ is bromine, X is NR¹R² and R¹ and R² for a particular compound correspond in each case to one row of Table A

### Table 197

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is chlorine, R⁸ is methyl, X is NR¹R² and R¹ and R² for a particular compound correspond in each case to one row of Table A

### Table 198

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is chlorine, R⁸ is methoxy, X is NR¹R² and R¹ and R² for a particular compound correspond in each case to one row of Table A

### Table 199

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is bromine, R⁸ is chlorine, X is NR¹R² and R¹ and R² for a particular compound correspond in each case to one row of Table A

### Table 200

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is bromine, R⁸ is bromine, X is NR¹R² and R¹ and R² for a particular compound correspond in each case to one row of Table A

### Table 201

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is bromine, R⁸ is methyl, X is NR¹R² and R¹ and R² for a particular compound correspond in each case to one row of Table A

### Table 202

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is bromine, R⁸ is methoxy, X is NR¹R² and R¹ and R² for a particular compound correspond in each case to one row of Table A

### Table 203

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is methyl, R⁸ is chlorine, X is NR¹R² and R¹ and R² for a particular compound correspond in each case to one row of Table A

### Table 204

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is methyl, R⁸ is bromine, X is NR¹R² and R¹ and R² for a particular compound correspond in each case to one row of Table A

### Table 205

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is methyl, R⁸ is methyl, X is NR¹R² and R¹ and R² for a particular compound correspond in each case to one row of Table A

### Table 206

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is methyl, R⁸ is methoxy, X is NR¹R² and R¹ and R² for a particular compound correspond in each case to one row of Table A

### Table 207

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is methoxy, R⁸ is chlorine, X is NR¹R² and R¹ and R² for a particular compound correspond in each case to one row of Table A

### Table 208

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is methoxy, R⁸ is bromine, X is NR¹R² and R¹ and R² for a particular compound correspond in each case to one row of Table A

### Table 209

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is methoxy, R⁸ is methyl, X is NR¹R² and R¹ and R² for a particular compound correspond in each case to one row of Table A

### Table 210

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶, R⁷, R⁸ are each methoxy, X is NR¹R² and R¹ and R² for a particular compound correspond in each case to one row of Table A

### Table 211

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is methyl, R⁶ is chlorine, R⁹ is methoxy, X is NR¹R² and R¹ and R² for a particular compound correspond in each case to one row of Table A

### Table 212

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is methyl, R⁶ is bromine, R⁹ is methoxy, X is NR¹R² and R¹ and R² for a particular compound correspond in each case to one row of Table A

### Table 213

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is methyl, R⁶ is methyl, R⁹ is methoxy, X is NR¹R² and R¹ and R² for a particular compound correspond in each case to one row of Table A

### Table 214

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is methyl, R⁶ is chlorine, R⁹ is methyl, X is NR¹R² and R¹ and R² for a particular compound correspond in each case to one row of Table A

### Table 215

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R¹ is methyl, R⁶ is bromine, R⁹ is methyl, X is NR¹R² and R¹ and R² for a particular cornpound correspond in each case to one row of Table A

### Table 216

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R¹ is methyl, R⁶ is methyl, R⁹ is methyl, X is NR¹R² and R¹ and R² for a particular compound correspond in each case to one row of Table A

### Table 217

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R¹ is methyl, R⁶ is chlorine, R⁹ is chlorine, X is NR¹R² and R¹ and R² for a particular compound correspond in each case to one row of Table A

### Table 218

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R¹ is methyl, R⁶ is bromine, R⁹ is chlorine, X is NR¹R² and R¹ and R² for a particular compound correspond in each case to one row of Table A

### Table 219

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is methyl, R⁶ is methyl, R⁹ is chlorine, X is NR¹R² and R¹ and R² for a particular compound correspond in each case to one row of Table A

### Table 220

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶, R⁷, R⁸ are each chlorine and X for a particular compound corresponds in each case to one row of Table B

### Table 221

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is chlorine, R⁸ is bromine and X for a particular compound corresponds in each case to one row of Table B

### Table 222

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is chlorine, R⁸ is methyl and X for a particular compound corresponds in each case to one row of Table B

### Table 223

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is chlorine, R⁸ is methoxy and X for a particular compound corresponds in each case to one row of Table B

### Table 224

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is bromine, R⁸ is chlorine and X for a particular compound corresponds in each case to one row of Table B

### Table 225

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is bromine, R⁸ is bromine and X for a particular compound corresponds in each case to one row of Table B

### Table 226

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is bromine, R⁸ is methyl and X for a particular compound corresponds in each case to one row of Table B

### Table 227

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is bromine, R⁸ is methoxy and X for a particular compound corresponds in each case to one row of Table B

### Table 228

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is methyl, R⁸ is chlorine and X for a particular compound corresponds in each case to one row of Table B

### Table 229

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is methyl, R⁸ is bromine and X for a particular compound corresponds in each case to one row of Table B

### Table 230

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is methyl, R⁸ is methyl and X for a particular compound corresponds in each case to one row of Table B

### Table 231

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is methyl, R⁸ is methoxy and X for a particular compound corresponds in each case to one row of Table B

### Table 232

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is methoxy, R⁸ is chlorine and X for a particular compound corresponds in each case to one row of Table B

### Table 233

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is methoxy, R⁸ is bromine and X for a particular compound corresponds in each case to one row of Table B

### Table 234

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is methoxy, R⁸ is methyl and X for a particular compound corresponds in each case to one row of Table B

### Table 235

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is chlorine, R⁷ is methoxy, R⁸ is methoxy and X for a particular compound corresponds in each case to one row of Table B

### Table 236

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is chlorine, R⁸ is chlorine and X for a particular compound corresponds in each case to one row of Table B

### Table 237

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is chlorine, R⁸ is bromine and X for a particular compound corresponds in each case to one row of Table B

### Table 238

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is chlorine, R⁸ is methyl and X for a particular compound corresponds in each case to one row of Table B

### Table 239

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is chlorine, R⁸ is methoxy and X for a particular compound corresponds in each case to one row of Table B

### Table 240

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is bromine, R⁸ is chlorine and X for a particular compound corresponds in each case to one row of Table B

### Table 241

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶, R⁷, R⁸ are each bromine and X for a particular compound corresponds in each case to one row of Table B

### Table 242

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is bromine, R⁸ is methyl and X for a particular compound corresponds in each case to one row of Table B

### Table 243

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is bromine, R⁸ is methoxy and X for a particular compound corresponds in each case to one row of Table B

### Table 244

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is methyl, R⁸ is chlorine and X for a particular compound corresponds in each case to one row of Table B

### Table 245

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is methyl, R⁸ is bromine and X for a particular compound corresponds in each case to one row of Table B

### Table 246

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is methyl, R⁸ is methyl and X for a particular compound corresponds in each case to one row of Table B

### Table 247

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is methyl, R⁸ is methoxy and X for a particular compound corresponds in each case to one row of Table B

### Table 248

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is methoxy, R⁸ is chlorine and X for a particular compound corresponds in each case to one row of Table B

### Table 249

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is methoxy, R⁸ is bromine and X for a particular compound corresponds in each case to one row of Table B

### Table 250

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is methoxy, R⁸ is methyl and X for a particular compound corresponds in each case to one row of Table B

### Table 251

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is bromine, R⁷ is methoxy, R⁸ is methoxy and X for a particular compound corresponds in each case to one row of Table B

### Table 252

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is chlorine, R⁸ is chlorine and X for a particular compound corresponds in each case to one row of Table B

### Table 253

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is chlorine, R⁸ is bromine and X for a particular compound corresponds in each case to one row of Table B

### Table 254

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is chlorine, R⁸ is methyl and X for a particular compound corresponds in each case to one row of Table B

### Table 255

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is chlorine, R⁸ is methoxy and X for a particular compound corresponds in each case to one row of Table B

### Table 256

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is bromine, R⁸ is chlorine and X for a particular compound corresponds in each case to one row of Table B

### Table 257

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is bromine, R⁸ is bromine and X for a particular compound corresponds in each case to one row of Table B

### Table 258

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is bromine, R⁸ is methyl and X for a particular compound corresponds in each case to one row of Table B

### Table 259

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is bromine, R⁸ is methoxy and X for a particular compound corresponds in each case to one row of Table B

### Table 260

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is methyl, R⁸ is chlorine and X for a particular compound corresponds in each case to one row of Table B

### Table 261

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is methyl, R⁸ is bromine and X for a particular compound corresponds in each case to one row of Table B

### Table 262

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶, R⁷, R⁸ are each methyl and X for a particular compound corresponds in each case to one row of Table B

### Table 263

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is methyl, R⁸ is methoxy and X for a particular compound corresponds in each case to one row of Table B

### Table 264

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is methoxy, R⁸ is chlorine and X for a particular compound corresponds in each case to one row of Table B

### Table 265

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is methoxy, R⁸ is bromine and X for a particular compound corresponds in each case to one row of Table B

### Table 266

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is methoxy, R⁸ is methyl and X for a particular compound corresponds in each case to one row of Table B

### Table 267

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methyl, R⁷ is methoxy, R⁸ is methoxy and X for a particular compound corresponds in each case to one row of Table B

### Table 268

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is chlorine, R⁸ is chlorine and X for a particular compound corresponds in each case to one row of Table B

### Table 269

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is chlorine, R⁸ is bromine and X for a particular compound corresponds in each case to one row of Table B

### Table 270

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is chlorine, R⁸ is methyl and X for a particular compound corresponds in each case to one row of Table B

### Table 271

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is chlorine, R⁸ is methoxy and X for a particular compound corresponds in each case to one row of Table B

### Table 272

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is bromine, R⁸ is chlorine and X for a particular compound corresponds in each case to one row of Table B

### Table 273

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is bromine, R⁸ is bromine and X for a particular compound corresponds in each case to one row of Table B

### Table 274

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is bromine, R⁸ is methyl and X for a particular compound corresponds in each case to one row of Table B

### Table 275

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is bromine, R⁸ is methoxy and X for a particular compound corresponds in each case to one row of Table B

### Table 276

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is methyl, R⁸ is chlorine and X for a particular compound corresponds in each case to one row of Table B

### Table 277

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is methyl, R⁸ is bromine and X for a particular compound corresponds in each case to one row of Table B

### Table 278

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is methyl, R⁸ is methyl and X for a particular compound corresponds in each case to one row of Table B

### Table 279

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is methyl, R⁸ is methoxy and X for a particular compound corresponds in each case to one row of Table B

### Table 280

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is methoxy, R⁸ is chlorine and X for a particular compound corresponds in each case to one row of Table B

### Table 281

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is methoxy, R⁸ is bromine and X for a particular compound corresponds in each case to one row of Table B

### Table 282

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶ is methoxy, R⁷ is methoxy, R⁸ is methyl and X for a particular compound corresponds in each case to one row of Table B

### Table 283

Compounds of the formulae I.1, I.2, I.3, I.4, I.5, I.6, I.7, I.8, I.9, I.10, I.11, I.12, I.13, I.14, I.15, I.16, I.17, I.18, I.19, I.20, I.21, I.22, I.23 and I.24 in which R⁵ is methyl, R⁶, R⁷, R⁸ are each methoxy and X for a particular compound corresponds in each case to one row of Table B

### Table 284

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁶ is methyl, R⁶ is chlorine, R⁹ is methoxy and X for a particular compound corresponds in each case to one row of Table B

### Table 285

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is methyl, R⁶ is bromine, R⁹ is methoxy and X for a particular compound corresponds in each case to one row of Table B

### Table 286

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is methyl, R⁶ is methyl, R⁹ is methoxy and X for a particular compound corresponds in each case to one row of Table B

### Table 287

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is methyl, R⁶ is chlorine, R⁹ is methyl and X for a particular compound corresponds in each case to one row of Table B

### Table 288

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is methyl, R⁶ is bromine, R⁹ is methyl and X for a particular compound corresponds in each case to one row of Table B

### Table 289

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is methyl, R⁶ is methyl, R⁹ is methyl and X for a particular compound corresponds in each case to one row of Table B

### Table 290

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is methyl, R⁶ is chlorine, R⁹ is chlorine and X for a particular compound corresponds in each case to one row of Table B

### Table 291

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is methyl, R⁶ is bromine, R⁹ is chlorine and X for a particular compound corresponds in each case to one row of Table B

### Table 292

Compounds of the formulae I.25, I.26, I.27, I.28, I.29, I.30, I.31, I.32, I.33, I.34, I.35, I.36, I.37, I.38, I.39, I.40, I.41, I.42, I.43, I.44, I.45, I.46, I.47 and I.48 in which R⁵ is methyl, R⁶ is methyl, R⁹ is chlorine and X for a particular compound corresponds in each case to one row of Table B

TABLE A

| Row | R¹ | R² |
|---|---|---|
| A-1 | H | H |
| A-2 | CH₃ | H |
| A-3 | CH₃ | CH₃ |
| A-4 | CH₃ | OCH₃ |
| A-5 | CH₂CH₃ | H |
| A-6 | CH₂CH₃ | CH₃ |
| A-7 | CH₂CH₃ | CH₂CH₃ |
| A-8 | CH₂CH₃ | OCH₃ |
| A-9 | CH₂CF₃ | H |
| A-10 | CH₂CF₃ | CH₃ |
| A-11 | CH₂CF₃ | CH₂CH₃ |
| A-12 | CH₂CF₃ | OCH₃ |
| A-13 | CH₂CCl₃ | H |
| A-14 | CH₂CCl₃ | CH₃ |
| A-15 | CH₂CCl₃ | CH₂CH₃ |
| A-16 | CH₂CCl₃ | OCH₃ |
| A-17 | CH₂CH₂CH₃ | H |
| A-18 | CH₂CH₂CH₃ | CH₃ |
| A-19 | CH₂CH₂CH₃ | CH₂CH₃ |
| A-20 | CH₂CH₂CH₃ | CH₂CH₂CH₃ |
| A-21 | CH₂CH₂CH₃ | OCH₃ |
| A-22 | CH(CH₃)₂ | H |
| A-23 | CH(CH₃)₂ | CH₃ |
| A-24 | CH(CH₃)₂ | CH₂CH₃ |
| A-25 | CH(CH₃)₂ | OCH₃ |
| A-26 | CH₂CH(CH₃)₂ | H |
| A-27 | CH₂CH(CH₃)₂ | CH₃ |
| A-28 | CH₂CH(CH₃)₂ | CH₂CH₃ |
| A-29 | CH₂CH(CH₃)₂ | CH₂CH₂CH₃ |
| A-30 | CH₂CH(CH₃)₂ | OCH₃ |
| A-31 | CH₂CH₂CH₂CH₃ | H |
| A-32 | CH₂CH₂CH₂CH₃ | CH₃ |
| A-33 | CH₂CH₂CH₂CH₃ | CH₂CH₃ |
| A-34 | CH₂CH₂CH₂CH₃ | CH₂CH₂CH₃ |
| A-35 | CH₂CH₂CH₂CH₃ | CH₂CH₂CH₂CH₃ |
| A-36 | CH₂CH₂CH₂CH₃ | OCH₃ |
| A-37 | (±)CH(CH₃)—CH₂CH₃ | H |
| A-38 | (±)CH(CH₃)—CH₂CH₃ | CH₃ |
| A-39 | (±)CH(CH₃)—CH₂CH₃ | CH₂CH₃ |
| A-40 | (±)CH(CH₃)—CH₂CH₃ | CH₂CH₃ |
| A-41 | (S)CH(CH₃)—CH₂CH₃ | H |
| A-42 | (S)CH(CH₃)—CH₂CH₃ | CH₃ |
| A-43 | (S)CH(CH₃)—CH₂CH₃ | CH₂CH₃ |
| A-44 | (S)CH(CH₃)—CH₂CH₃ | OCH₃ |
| A-45 | (R)CH(CH₃)—CH₂CH₃ | H |
| A-46 | (R)CH(CH₃)—CH₂CH₃ | CH₃ |
| A-47 | (R)CH(CH₃)—CH₂CH₃ | CH₂CH₃ |
| A-48 | (R)CH(CH₃)—CH₂CH₃ | OCH₃ |
| A-49 | (±)CH(CH₃)—CH(CH₃)₂ | H |
| A-50 | (±)CH(CH₃)—CH(CH₃)₂ | CH₃ |
| A-51 | (±)CH(CH₃)—CH(CH₃)₂ | CH₂CH₃ |
| A-52 | (±)CH(CH₃)—CH(CH₃)₂ | OCH₃ |
| A-53 | (S)CH(CH₃)—CH(CH₃)₂ | H |
| A-54 | (S)CH(CH₃)—CH(CH₃)₂ | CH₃ |
| A-55 | (S)CH(CH₃)—CH(CH₃)₂ | CH₂CH₃ |
| A-56 | (S)CH(CH₃)—CH(CH₃)₂ | OCH₃ |
| A-57 | (R)CH(CH₃)—CH(CH₃)₂ | H |
| A-58 | (R)CH(CH₃)—CH(CH₃)₂ | CH₃ |
| A-59 | (R)CH(CH₃)—CH(CH₃)₂ | CH₂CH₃ |
| A-60 | (R)CH(CH₃)—CH(CH₃)₂ | OCH₃ |
| A-61 | (±)CH(CH₃)—C(CH₃)₃ | H |
| A-62 | (±)CH(CH₃)—C(CH₃)₃ | CH₃ |
| A-63 | (±)CH(CH₃)—C(CH₃)₃ | CH₂CH₃ |
| A-64 | (±)CH(CH₃)—C(CH₃)₃ | OCH₃ |
| A-65 | (S)CH(CH₃)—C(CH₃)₃ | H |
| A-66 | (S)CH(CH₃)—C(CH₃)₃ | CH₃ |
| A-67 | (S)CH(CH₃)—C(CH₃)₃ | CH₂CH₃ |
| A-68 | (S)CH(CH₃)—C(CH₃)₃ | OCH₃ |
| A-69 | (R)CH(CH₃)—C(CH₃)₃ | H |
| A-70 | (R)CH(CH₃)—C(CH₃)₃ | CH₃ |
| A-71 | (R)CH(CH₃)—C(CH₃)₃ | CH₂CH₃ |
| A-72 | (R)CH(CH₃)—C(CH₃)₃ | OCH₃ |
| A-73 | (±)CH(CH₃)—CF₃ | H |
| A-74 | (±)CH(CH₃)—CF₃ | CH₃ |
| A-75 | (±)CH(CH₃)—CF₃ | CH₂CH₃ |
| A-76 | (±)CH(CH₃)—CF₃ | OCH₃ |
| A-77 | (S)CH(CH₃)—CF₃ | H |
| A-78 | (S)CH(CH₃)—CF₃ | CH₃ |
| A-79 | (S)CH(CH₃)—CF₃ | CH₂CH₃ |
| A-80 | (S)CH(CH₃)—CF₃ | OCH₃ |
| A-81 | (R)CH(CH₃)—CF₃ | H |
| A-82 | (R)CH(CH₃)—CF₃ | CH₃ |
| A-83 | (R)CH(CH₃)—CF₃ | CH₂CH₃ |
| A-84 | (R)CH(CH₃)—CF₃ | OCH₃ |
| A-85 | (±)CH(CH₃)—CCl₃ | H |
| A-86 | (±)CH(CH₃)—CCl₃ | CH₃ |
| A-87 | (±)CH(CH₃)—CCl₃ | CH₂CH₃ |
| A-88 | (±)CH(CH₃)—CCl₃ | OCH₃ |
| A-89 | (S)CH(CH₃)—CCl₃ | H |
| A-90 | (S)CH(CH₃)—CCl₃ | CH₃ |
| A-91 | (S)CH(CH₃)—CCl₃ | CH₂CH₃ |
| A-92 | (S)CH(CH₃)—CCl₃ | OCH₃ |
| A-93 | (R)CH(CH₃)—CCl₃ | H |
| A-94 | (R)CH(CH₃)—CCl₃ | CH₃ |
| A-95 | (R)CH(CH₃)—CCl₃ | CH₂CH₃ |
| A-96 | (R)CH(CH₃)—CCl₃ | OCH₃ |
| A-97 | CH₂CF₂CF₃ | H |
| A-98 | CH₂CF₂CF₃ | CH₃ |
| A-99 | CH₂CF₂CF₃ | CH₂CH₃ |
| A-100 | CH₂CF₂CF₃ | OCH₃ |
| A-101 | CH₂(CF₂)₂CF₃ | H |
| A-102 | CH₂(CF₂)₂CF₃ | CH₃ |
| A-103 | CH₂(CF₂)₂CF₃ | CH₂CH₃ |
| A-104 | CH₂(CF₂)₂CF₃ | OCH₃ |
| A-105 | CH₂C(CH₃)═CH₂ | H |
| A-106 | CH₂C(CH₃)═CH₂ | CH₃ |
| A-107 | CH₂C(CH₃)═CH₂ | CH₂CH₃ |
| A-108 | CH₂C(CH₃)═CH₂ | OCH₃ |
| A-109 | CH₂CH═CH₂ | H |
| A-110 | CH₂CH═CH₂ | CH₃ |
| A-111 | CH₂CH═CH₂ | CH₂CH₃ |
| A-112 | CH₂CH═CH₂ | OCH₃ |
| A-113 | (±)CH(CH₃)CH═CH₂ | H |
| A-114 | (±)CH(CH₃)CH═CH₂ | CH₃ |
| A-115 | (±)CH(CH₃)CH═CH₂ | CH₂CH₃ |
| A-116 | (±)CH(CH₃)CH═CH₂ | OCH₃ |
| A-117 | (S)CH(CH₃)CH═CH₂ | H |
| A-118 | (S)CH(CH₃)CH═CH₂ | CH₃ |
| A-119 | (S)CH(CH₃)CH═CH₂ | CH₂CH₃ |
| A-120 | (S)CH(CH₃)CH═CH₂ | OCH₃ |
| A-121 | (R)CH(CH₃)CH═CH₂ | H |
| A-122 | (R)CH(CH₃)CH═CH₂ | CH₃ |
| A-123 | (R)CH(CH₃)CH═CH₂ | CH₂CH₃ |
| A-124 | (R)CH(CH₃)CH═CH₂ | OCH₃ |
| A-125 | (±)CH(CH₃)CH═CH₂ | H |
| A-126 | (±)CH(CH₃)CH═CH₂ | CH₃ |
| A-127 | (±)CH(CH₃)CH═CH₂ | CH₂CH₃ |
| A-128 | (±)CH(CH₃)CH═CH₂ | OCH₃ |
| A-129 | (S)CH(CH₃)CH═CH₂ | H |
| A-130 | (S)CH(CH₃)CH═CH₂ | CH₃ |
| A-131 | (S)CH(CH₃)CH═CH₂ | CH₂CH₃ |
| A-132 | (S)CH(CH₃)CH═CH₂ | OCH₃ |
| A-133 | (R)CH(CH₃)CH═CH₂ | H |
| A-134 | (R)CH(CH₃)CH═CH₂ | CH₃ |
| A-135 | (R)CH(CH₃)CH═CH₂ | CH₂CH₃ |
| A-136 | (R)CH(CH₃)CH═CH₂ | OCH₃ |
| A-137 | CH(CH₃)C(CH₃)═CH₂ | H |
| A-138 | CH(CH₃)C(CH₃)═CH₂ | CH₃ |
| A-139 | CH(CH₃)C(CH₃)═CH₂ | CH₂CH₃ |
| A-140 | CH(CH₃)C(CH₃)═CH₂ | OCH₃ |
| A-141 | CH₂—C≡CH | H |
| A-142 | CH₂—C≡CH | CH₃ |
| A-143 | CH₂—C≡CH | CH₂CH₃ |
| A-144 | CH₂—C≡CH | OCH₃ |
| A-145 | cyclopentyl | H |
| A-146 | cyclopentyl | CH₃ |
| A-147 | cyclopentyl | CH₂CH₃ |
| A-148 | cyclopentyl | OCH₃ |
| A-149 | cyclohexyl | H |
| A-150 | cyclohexyl | CH₃ |
| A-151 | cyclohexyl | CH₂CH₃ |
| A-152 | cyclohexyl | OCH₃ |
| A-153 | CH₂-(c-C₃H₅) | H |
| A-154 | CH₂-(c-C₃H₅) | CH₃ |
| A-155 | CH₂-(c-C₃H₅) | CH₂CH₃ |
| A-156 | CH₂-(c-C₃H₅) | OCH₃ |
| A-157 | CH₂—C₆H₅ | H |
| A-158 | CH₂—C₆H₅ | CH₃ |
| A-159 | CH₂—C₆H₅ | CH₂CH₃ |
| A-160 | CH₂—C₆H₅ | OCH₃ |
| A-161 | NH₂ | CH₂-c-C₆H₁₁ |
| A-162 | NH₂ | CH₂CH₃ |
| A-163 | NH₂ | CH₂CH₂CH₃ |
| A-164 | NH₂ | OCH₃ |
| A-165 | NH—CH₂—CH═CH₂ | H |
| A-166 | NH—CH₂—CH═CH₂ | CH₃ |
| A-167 | NH—CH₂—CH═CH₂ | CH₂CH₃ |
| A-168 | NH—CH₂—CH═CH₂ | OCH₃ |
| A-169 | NH—C(CH₃)₃ | H |
| A-170 | NH—C(CH₃)₃ | OCH₃ |
| A-171 | N(CH₃)₂ | H |
| A-172 | N(CH₃)₂ | OCH₃ |
| A-173 | NH(CH₃) | H |
| A-174 | NH(CH₃) | OCH₃ |

| | |
|---|---|
| A-175 | —(CH₂)₂CH═CHCH₂— |
| A-176 | —(CH₂)₂C(CH₃)═CHCH₂— |
| A-177 | —CH(CH₃)CH₂—CH═CHCH₂— |
| A-178 | —(CH₂)₂CH(CH₃)(CH₂)₂— |
| A-179 | —(CH₂)₃CHFCH₂— |
| A-180 | —(CH₂)₂CHF(CH₂)₂— |
| A-181 | —CH₂CHF(CH₂)₃— |
| A-182 | —(CH₂)₂CH(CF₃)(CH₂)₂— |
| A-183 | —(CH₂)₂O(CH₂)₂— |
| A-184 | —(CH₂)₂S(CH₂)₂— |
| A-185 | —OCH(CH₃)(CH₂)₃— |
| A-186 | —OCH₂CH(CH₃)(CH₂)₂— |
| A-187 | —(CH₂)₅— |
| A-188 | —(CH₂)₄— |
| A-189 | —CH₂CH═CHCH₂— |
| A-190 | —CH(CH₃)(CH₂)₃— |
| A-191 | —CH₂CH(CH₃)(CH₂)₂— |
| A-192 | —CH(CH₃)—(CH₂)₂—CH(CH₃)— |
| A-193 | —CH(CH₃)—(CH₂)₄— |
| A-194 | —CH₂—CH(CH₃)—(CH₂)₃— |
| A-195 | —(CH₂)—CH(CH₃)—CH₂—CH(CH₃)—CH₂— |
| A-196 | —CH(CH₂CH₃)—(CH₂)₄— |
| A-197 | —(CH₂)₂—CHOH—(CH₂)₂— |
| A-198 | —(CH₂)₆— |
| A-199 | —CH(CH₃)—(CH₂)₅— |
| A-200 | —(CH₂)₂—N(CH₃)—(CH₂)₂— |

TABLE B

| Row | X |
|---|---|
| B-1 | OCH₂CH |
| B-2 | OCH₂CH₂CH₃ |
| B-3 | OCH₂CH₂F |
| B-4 | OCH₂CF₃ |
| B-5 | OCH₂CCl₃ |
| B-6 | OCH(CH₃)₂ |
| B-7 | OCH₂C(CH₃)₃ |
| B-8 | OCH₂CH(CH₃)₂ |
| B-9 | (±)OCH(CH₂CH₃)CH₃ |
| B-10 | (R)OCH(CH₂CH₃)CH₃ |
| B-11 | (S)OCH(CH₂CH₃)CH₃ |
| B-12 | (±)OCH(CH₃)—CH(CH₃)₂ |
| B-13 | (R)OCH(CH₃)—CH(CH₃)₂ |
| B-14 | (S)OCH(CH₃)—CH(CH₃)₂ |
| B-15 | (±)OCH(CH₃)—C(CH₃)₃ |
| B-16 | (R)OCH(CH₃)—C(CH₃)₃ |
| B-17 | (S)OCH(CH₃)—C(CH₃)₃ |
| B-18 | (±)OCH(CH₃)—CF₃ |
| B-19 | (R)OCH(CH₃)—CF₃ |
| B-20 | (S)OCH(CH₃)—CF₃ |
| B-21 | (±)OCH(CH₃)—CCl₃ |
| B-22 | (R)OCH(CH₃)—CCl₃ |
| B-23 | (S)OCH(CH₃)—CCl₃ |
| B-24 | OCH₂C(CH₃)═CH₂ |
| B-25 | O-Cyclopentyl |
| B-26 | O-Cyclohexyl |
| B-27 | O(CH₂)₃CH₃ |
| B-28 | OC(CH₃)₃ |
| B-29 | O(CH₂)₄CH₃ |
| B-30 | OCH(CH₂CH₃)₂ |
| B-31 | OCH₂CH₂CH(CH₃)₂ |
| B-32 | (±)OCH(CH₃)(CH₂)₂CH₃ |
| B-33 | (R)OCH(CH₃)(CH₂)₂CH₃ |
| B-34 | (S)OCH(CH₃)(CH₂)₂CH₃ |
| B-35 | (±)OCH₂CH(CH₃)CH₂CH₃ |
| B-36 | (R)OCH₂CH(CH₃)CH₂CH₃ |
| B-37 | (S)OCH₂CH(CH₃)CH₂CH₃ |
| B-38 | (±)OCH(CH₃)CH(CH₃)₂ |
| B-39 | (R)OCH(CH₃)CH(CH₃)₂ |
| B-40 | (S)OCH(CH₃)CH(CH₃)₂ |
| B-41 | O(CH₂)₅CH₃ |
| B-42 | (±,±)OCH(CH₃)CH(CH₃)CH₂CH₃ |
| B-43 | (±,R)OCH(CH₃)CH(CH₃)CH₂CH₃ |
| B-44 | (±,S)OCH(CH₃)CH(CH₃)CH₂CH₃ |
| B-45 | (±)OCH₂CH(CH₃)CF₃ |
| B-46 | (R)OCH₂CH(CH₃)CF₃ |
| B-47 | (S)OCH₂CH(CH₃)CF₃ |
| B-48 | (±)OCH₂CH(CF₃)CH₂CH₃ |
| B-49 | (R)OCH₂CH(CF₃)CH₂CH₃ |
| B-50 | (S)OCH₂CH(CF₃)CH₂CH₃ |
| B-51 | (±,±)OCH(CH₃)CH(CH₃)CF₃ |
| B-52 | (±,R)OCH(CH₃)CH(CH₃)CF₃ |
| B-53 | (±,S)OCH(CH₃)CH(CH₃)CF₃ |
| B-54 | (±,±)OCH(CH₃)CH(CF₃)CH₂CH₃ |
| B-55 | (±,R)OCH(CH₃)CH(CF₃)CH₂CH₃ |
| B-56 | (±,S)OCH(CH₃)CH(CF₃)CH₂CH₃ |
| B-57 | OCF₃ |
| B-58 | OCF₂CF₃ |
| B-59 | OCF₂CF₂CF₃ |
| B-60 | O-(c-C₃H₅) |
| B-61 | O-(1-CH₃)-c-C₃H₄ |
| B-62 | O-(c-C₅H₉) |
| B-63 | O-(c-C₆H₁₁) |
| B-64 | O-(4-CH₃)-c-C₆H₁₀ |
| B-65 | OCH₂C(CH₃)═CH₂ |
| B-66 | OCH₂CH₂C(CH₃)═CH₂ |
| B-67 | OCH₂—C(CH₃)₃ |
| B-68 | OCH₂—Si(CH₃)₃ |
| B-69 | O-(n-C₆H₁₃) |
| B-70 | O(CH₂)₃—CH(CH₃)₂ |
| B-71 | O(CH₂)₂—CH(CH₃)—C₂H₅ |
| B-72 | OCH₂—CH(CH₃)-n-C₃H₇ |
| B-73 | OCH(CH₃)-n-C₄H₉ |
| B-74 | OCH₂—CH(C₂H₅)₂ |
| B-75 | OCH(C₂H₅)-n-C₃H₇ |
| B-76 | OCH₂-c-C₅H₉ |
| B-77 | OCH₂—CH(CH₃)—CH(CH₃)₂ |
| B-78 | OCH(CH₃)—CH₂CH(CH₃)₂ |
| B-79 | OCH(CH₃)—CH(CH₃)—C₂H₅ |
| B-80 | OCH(CH₃)—C(CH₃)₃ |
| B-81 | O(CH₂)₂—C(CH₃)₃ |
| B-82 | OCH₂—C(CH₃)₂—C₂H₅ |
| B-83 | O-(2-CH₃-c-C₅H₈) |
| B-84 | O-(3-CH₃-c-C₅H₈) |
| B-85 | OC(CH₃)₂-n-C₃H₇ |
| B-86 | O(CH₂)₆—CH₃ |
| B-87 | O(CH₂)₄—CH(CH₃)₂ |
| B-88 | O(CH₂)₃—CH(CH₃)—C₂H₅ |
| B-89 | O(CH₂)₂—CH(CH₃)-n-C₃H₇ |
| B-90 | OCH₂—CH(CH₃)-n-C₄H₉ |
| B-91 | OCH(CH₃)-n-C₅H₁₁ |
| B-92 | O(CH₂)₃C(CH₃)₃ |
| B-93 | O(CH₂)₂CH(CH₃)—CH(CH₃)₂ |
| B-94 | O(CH₂)CH(CH₃)—CH₂CH(CH₃)₂ |
| B-95 | OCH(CH₃)(CH₂)₂—CH(CH₃)₂ |
| B-96 | O(CH₂)₂C(CH₃)₂C₂H₅ |
| B-97 | OCH₂CH(CH₃)CH(CH₃)C₂H₅ |
| B-98 | OCH(CH₃)CH₂CH(CH₃)C₂H₅ |
| B-99 | OCH₂C(CH₃)₂-n-C₃H₇ |
| B-100 | OCH(CH₃)CH(CH₃)-n-C₃H₇ |
| B-101 | OC(CH₃)₂-n-C₄H₉ |
| B-102 | O(CH₂)₂CH(C₂H₅)₂ |
| B-103 | OCH₂CH(C₂H₅)-n-C₃H₇ |
| B-104 | OCH(02H₅)-n-C₄H₉ |
| B-105 | OCH₂CH(CH₃)C(CH₃)₃ |
| B-106 | OCH(CH₃)CH₂C(CH₃)₃ |
| B-107 | OCH₂C(CH₃)₂CH(CH₃)₂ |
| B-108 | OCH₂CH(C₂H₅)CH(CH₃)₂ |
| B-109 | OCH(CH₃)CH(CH₃)CH(CH₃)₂ |
| B-110 | OC(CH₃)₂CH₂CH(CH₃)₂ |
| B-111 | OCH(C₂H₅)CH₂CH(CH₃)₂ |
| B-112 | OCH(CH₃)C(CH₃)₂C₂H₅ |
| B-113 | OCH(CH₃)CH(C₂H₅)₂ |
| B-114 | OC(CH₃)₂CH(CH₃)C₂H₅ |
| B-115 | OCH(O₂H₅)CH(CH₃)C₂H₅ |
| B-116 | OC(CH₃)(C₂H₅)-n-C₃H₇ |
| B-117 | OCH(n-C₃H₇)₂ |
| B-118 | OCH(n-C₃H₇)CH(CH₃)₂ |
| B-119 | OC(CH₃)₂C(CH₃)₃ |
| B-120 | OC(CH₃)(C₂H₅)—CH(CH₃)₂ |
| B-121 | OC(C₂H₅)₃ |
| B-122 | O—(3—CH₃)c-C₆H₁₀) |
| B-123 | O—(2—CH₃-c-C₆H₁₀) |
| B-124 | O-n-C₈H₁₇ |
| B-125 | OCH═CH—CH₂CH₃ |
| B-126 | OCH₂—CH═CH—CH₃ |
| B-127 | OCH₂—CH₂—CH═CH₂ |
| B-128 | OC(CH₃)₂CH₂CH₃ |
| B-129 | OCH═C(CH₃)₂ |
| B-130 | OC(═CH₂)—CH₂CH₃ |
| B-131 | OC(CH₃)═CH—CH₃ |
| B-132 | OCH(CH₃)CH═CH₂ |
| B-133 | OCH═CH-n-C₃H₇ |
| B-134 | OCH₂—CH═CH—C₂H₅ |
| B-135 | O(CH₂)₂—CH═CH—CH₃ |
| B-136 | O(CH₂)₃—CH═CH₂ |
| B-137 | OCH═CH—CH(CH₃)₂ |
| B-138 | OCH₂—CH═C(CH₃)₂ |
| B-139 | O(CH₂)₂—C(CH₃)═CH₂ |
| B-140 | OCH═C(CH₃)—C₂H₅ |
| B-141 | OCH₂—C(═CH₂)—C₂H₅ |
| B-142 | OCH₂—C(CH₃)═CH—CH₃ |
| B-143 | OCH₂—CH(CH₃)—CH═CH₂ |
| B-144 | OC(═CH₂)—CH₂—CH₂—CH₃ |
| B-145 | OC(CH₃)═CH—CH₂—CH₃ |
| B-146 | OCH(CH₃)—CH═CH—CH₃ |
| B-147 | OCH(CH₃)—CH₂—CH═CH₂ |
| B-148 | OC(═CH₂)CH(CH₃)₂ |
| B-149 | OC(CH₃)═C(CH₃)₂ |
| B-150 | OCH(CH₃)—C(═CH₂)—CH₃ |
| B-151 | OC(CH₃)₂—CH═CH₂ |
| B-152 | OC(C₂H₅)═CH—CH₃ |
| B-153 | OCH(C₂H₅)—CH═CH₂ |
| B-154 | OCH═CH—CH₂—CH₂—CH₂—CH₃ |
| B-155 | OCH₂—CH═CH—CH₂—CH₂—CH₃ |
| B-156 | OCH₂—CH₂—CH═CH—CH₂—CH₃ |
| B-157 | OCH₂—CH₂—CH₂—CH═CH—CH₃ |
| B-158 | OCH₂—CH₂—CH₂—CH₂—CH═CH₂ |
| B-159 | OCH═CH—CH₂—CH(CH₃)CH₃ |
| B-160 | OCH₂—CH═CH—CH(CH₃)CH₃ |
| B-161 | OCH₂—CH₂—CH═C(CH₃)CH₃ |
| B-162 | OCH₂—CH₂—CH₂—C(CH₃)═CH₂ |
| B-163 | OCH═CH—CH(CH₃)—CH₂—CH₃ |
| B-164 | OCH₂—CH═C(CH₃)—CH₂—CH₃ |
| B-165 | OCH₂—CH₂—C(═CH₂)—CH₂—CH₃ |
| B-166 | OCH₂—CH₂—C(CH₃)═CH—CH₃ |
| B-167 | OCH₂—CH₂—CH(CH₃)—CH═CH₂ |
| B-168 | OCH═C(CH₃)—CH₂—CH₂—CH₃ |
| B-169 | OCH₂—C(═CH₂)—CH₂—CH₂—CH₃ |
| B-170 | OCH₂—C(CH₃)═CH—CH₂—CH₃ |
| B-171 | OCH₂—CH(CH₃)—CH═CH—CH₃ |
| B-172 | OCH₂—CH(CH₃)—CH₂—CH═CH₂ |
| B-173 | OC(═CH₂)—CH₂—CH₂—CH₂—CH₃ |
| B-174 | OC(CH₃)═CH—CH₂—CH₂—CH₃ |
| B-175 | OCH(CH₃)—CH═CH—CH₂—CH₃ |
| B-176 | OCH(CH₃)—CH₂—CH═CH—CH₃ |
| B-177 | OCH(CH₃)—CH₂—CH₂—CH═CH₂ |
| B-178 | OCH═CH—C(CH₃)₃ |
| B-179 | OCH═C(CH₃)—CH(CH₃)—CH₃ |
| B-180 | OCH₂—C(═CH₂)—CH(CH₃)—CH₃ |
| B-181 | OCH₂—C(CH₃)═C(CH₃)—CH₃ |
| B-182 | OCH₂—CH(CH₃)—C(═CH₂)—CH₃ |
| B-183 | OC(═CH₂)—CH₂—CH(CH₃)—CH₃ |
| B-184 | OC(CH₃)═CH—CH(CH₃)—CH₃ |
| B-185 | OCH(CH₃)—CH═C(CH₃)—CH₃ |
| B-186 | OCH(CH₃)—CH₂—C(═CH₂)—CH₃ |
| B-187 | OCH═C(CH₂—CH₃)—CH₂—CH₃ |
| B-188 | OCH₂—C(═CH—CH₃)—CH₂—CH₃ |
| B-189 | OCH₂—CH(CH═CH₂)—CH₂—CH₃ |
| B-190 | OC(═CH—CH₃)—CH₂—CH₂—CH₃ |
| B-191 | OCH(CH═CH₂)—CH₂—CH₂—CH₃ |
| B-192 | OC(CH₂—CH₃)═CH—CH₂—CH₃ |
| B-193 | OCH(CH₂—CH₃)—CH═CH—CH₃ |
| B-194 | OCH(CH₂—CH₃)—CH₂—CH═CH₂ |
| B-195 | OCH₂—C(CH₃)₂—CH═CH₂ |
| B-196 | OC(═CH₂)—CH(CH₃)—CH₂—CH₃ |
| B-197 | OC(CH₃)═C(CH₃)—CH₂—CH₃ |
| B-198 | OCH(CH₃)—C(═CH₂)—CH₂—CH₃ |
| B-199 | OCH(CH₃)—C(CH₃)═CH—CH₃ |
| B-200 | OCH(CH₃)—CH(CH₃)—CH═CH₂ |
| B-201 | OC(CH₃)₂—CH═CH—CH₃ |
| B-202 | OC(CH₃)₂—CH₂—CH═CH₂ |
| B-203 | OC(═CH₂)—C(CH₃)₃ |
| B-204 | OC(═CH—CH₃)—CH(CH₃)—CH₃ |
| B-205 | OCH(CH═CH₂)—CH(CH₃)—CH₃ |
| B-206 | OC(CH₂—CH₃)═C(CH₃)—CH₃ |
| B-207 | OCH(CH₂—CH₃)—C(═CH₂)—CH₃ |
| B-208 | OC(CH₃)₂—C(═CH₂)—CH₃ |
| B-209 | OC(CH₃)(CH═CH₂)—CH₂—CH₃ |
| B-210 | OC(CH₃)(CH₂CH₃)—CH₂—CH₂—CH₃ |
| B-211 | OCH(CH₂CH₃)—CH(CH₃)—CH₂—CH₃ |
| B-212 | OCH(CH₂CH₃)—CH₂—CH(CH₃)—CH₃ |
| B-213 | OC(CH₃)₂—C(CH₃)₃ |
| B-214 | OC(CH₂—CH₃)—C(CH₃)₃ |
| B-215 | OC(CH₃)(CH₂—CH₃)—CH(CH₃)₂ |
| B-216 | OCH(CH(CH₃)₂)—CH(CH₃)₂ |
| B-217 | O—CH═CH—CH₂—CH₂—CH₂—CH₂—CH₃ |
| B-218 | O—CH₂—CH═CH—CH₂—CH₂—CH₂—CH₃ |
| B-219 | O—CH₂—CH₂—CH═CH—CH₂—CH₂—CH₃ |
| B-220 | O—CH₂—CH₂—CH₂—CH═CH—CH₂—CH₃ |
| B-221 | O—CH₂—CH₂—CH₂—CH₂—CH═CH—CH₃ |
| B-222 | O—CH₂—CH₂—CH₂—CH₂—CH₂—CH═CH₂ |
| B-223 | O—CH═CH—CH₂—CH₂—CH(CH₃)—CH₃ |
| B-224 | O—CH₂—CH═CH—CH₂—CH(CH₃)—CH₃ |
| B-225 | O—CH₂—CH₂—CH═CH—CH(CH₃)—CH₃ |
| B-226 | O—CH₂—CH₂—CH₂—CH═C(CH₃)—CH₃ |
| B-227 | O—CH₂—CH₂—CH₂—CH₂—C(═CH₂)—CH₃ |
| B-228 | O—CH═CH—CH₂—CH(CH₃)—CH₂—CH₃ |
| B-229 | O—CH₂—CH═CH—CH(CH₃)—CH₂—CH₃ |
| B-230 | O—CH₂—CH₂—CH═C(CH₃)—CH₂—CH₃ |
| B-231 | O—CH₂—CH₂—CH₂—C(═CH₂)—CH₂—CH₃ |
| B-232 | O—CH₂—CH₂—CH₂—C(CH₃)═CH—CH₃ |
| B-233 | O—CH₂—CH₂—CH₂—CH(CH₃)—CH═CH₂ |
| B-234 | O—CH═CH—CH(CH₃)—CH₂—CH₂—CH₃ |
| B-235 | O—CH₂—CH═C(CH₃)—CH₂—CH₂—CH₃ |
| B-236 | O—CH₂—CH₂—C(═CH₂)—CH₂—CH₂—CH₃ |
| B-237 | O—CH₂—CH₂—C(CH₃)═CH—CH₂—CH₃ |
| B-238 | O—CH₂—CH₂—CH(CH₃)—CH═CH—CH₃ |
| B-239 | O—CH₂—CH₂—CH(CH₃)—CH₂—CH═CH₂ |
| B-240 | O—CH═C(CH₃)—CH₂—CH₂—CH₂—CH₃ |
| B-241 | O—CH₂—C(═CH₂)—CH₂—CH₂—CH₂—CH₃ |
| B-242 | O—CH₂—C(CH₃)═CH—CH₂—CH₂—CH₃ |
| B-243 | O—CH₂—CH(CH₃)—CH═CH—CH₂—CH₃ |
| B-244 | O—CH₂—CH(CH₃)—CH₂—CH═CH—CH₃ |
| B-245 | O—CH₂—CH(CH₃)—CH₂—CH₂—CH═CH₂ |
| B-246 | O—C(═CH₂)—CH₂—CH₂—CH₂—CH₂—CH₃ |
| B-247 | O—C(CH₃)═CH—CH₂—CH₂—CH₂—CH₃ |
| B-248 | O—CH(CH₃)—CH═CH—CH₂—CH₂—CH₃ |
| B-249 | O—CH(CH₃)—CH₂—CH═CH—CH₂—CH₃ |
| B-250 | O—CH(CH₃)—CH₂—CH₂—CH═CH—CH₃ |
| B-251 | O—CH(CH₃)—CH₂—CH₂—CH₂—CH═CH₂ |
| B-252 | O—CH═CH—CH₂—C(CH₃)₃ |
| B-253 | O—CH₂—CH═CH—C(CH₃)₃ |
| B-254 | O—CH═CH—CH(CH₃)—CH(CH₃)₂ |
| B-255 | O—CH₂—CH═C(CH₃)—CH(CH₃)₂ |
| B-256 | O—CH₂—CH₂—C(═CH₂)—CH(CH₃)₂ |
| B-257 | O—CH₂—CH₂—C(CH₃)═C(CH₃)₂ |
| B-258 | O—CH₂—CH₂—CH(CH₃)—C(═CH₂)—CH₃ |
| B-259 | O—CH═C(CH₃)—CH₂—CH(CH₃)₂ |
| B-260 | O—CH₂—C(═CH₂)—CH₂—CH(CH₃)₂ |
| B-261 | O—CH₂—C(CH₃)═CH—CH(CH₃)₂ |
| B-262 | O—CH₂—CH(CH₃)—CH═C(CH₃)₂ |
| B-263 | O—CH₂—CH(CH₃)—CH₂—C(═CH₂)—CH₃ |
| B-264 | O—C(═CH₂)—CH₂—CH₂—CH(CH₃)₂ |
| B-265 | O—C(CH₃)═CH—CH₂—CH(CH₃)₂ |
| B-266 | O—CH(CH₃)—CH═CH—CH(CH₃)₂ |
| B-267 | OCH(CH₃)—CH₂—CH═C(CH₃)₂ |
| B-268 | OCH(CH₃)—CH₂—CH₂—C(═CH₂)—CH₃ |
| B-269 | OCH═CH—C(CH₃)₂—CH₂—CH₃ |
| B-270 | OCH₂—CH₂—C(CH₃)₂—CH═CH₂ |
| B-271 | OCH═C(CH₃)—CH(CH₃)—CH₂—CH₃ |
| B-272 | OCH₂—C(═CH₂)—CH(CH₃)—CH₂—CH₃ |
| B-273 | OCH₂—C(CH₃)═C(CH₃)—CH₂—CH₃ |
| B-274 | OCH₂—CH(CH₃)—C(═CH₂)—CH₂—CH₃ |
| B-275 | OCH₂—CH(CH₃)—C(CH₃)═CH—CH₃ |
| B-276 | OCH₂—CH(CH₃)—CH(CH₃)—CH═CH₂ |
| B-277 | OC(═CH₂)—CH₂—CH(CH₃)—CH₂—CH₃ |
| B-278 | OC(CH₃)═CH—CH(CH₃)—CH₂—CH₃ |
| B-279 | OCH(CH₃)—CH═C(CH₃)—CH₂—CH₃ |
| B-280 | OCH(CH₃)—CH₂—C(═CH₂)—CH₂—CH₃ |
| B-281 | OCH(CH₃)—CH₂—C(CH₃)═CH—CH₃ |
| B-282 | OCH(CH₃)—CH₂—CH(CH₃)—CH═CH₂ |
| B-283 | OCH₂—C(CH₃)₂—CH═CH—CH₃ |
| B-284 | OCH₂—C(CH₃)₂—CH₂—CH═CH₂ |
| B-285 | OC(═CH₂)—CH(CH₃)—CH₂—CH₂—CH₃ |
| B-286 | OC(CH₃)═C(CH₃)—CH₂—CH₂—CH₃ |
| B-287 | OCH(CH₃)—C(═CH₂)—CH₂—CH₂—CH₃ |
| B-288 | OCH(CH₃)—C(CH₃)═CH—CH₂—CH₃ |
| B-289 | OCH(CH₃)—CH(CH₃)—CH═CH—CH₃ |
| B-290 | OCH(CH₃)—CH(CH₃)—CH₂—CH═CH₂ |
| B-291 | OC(CH₃)₂—CH═CH—CH₂—CH₃ |
| B-292 | OC(CHs)₂—CH₂—CH═CH—CH₃ |
| B-293 | OC(CH₃)₂—CH₂—CH₂—CH═CH₂ |
| B-294 | OCH═CH—CH(CH₂—CH₃)—CH₂—CH₃ |
| B-295 | OCH₂—CH═C(CH₂—CH₃)—CH₂—CH₃ |
| B-296 | OCH₂—CH₂—C(═CH—CH₃)—CH₂—CH₃ |
| B-297 | OCH₂—CH₂—CH(CH═CH₂)—CH₂—CH₃ |
| B-298 | OCH═C(CH₂—CH₃)—CH₂—CH₂—CH₃ |
| B-299 | OCH₂—C(═CH—CH₃)—CH₂—CH₂—CH₃ |
| B-300 | OCH₂—CH (CH═CH₂)—CH₂—CH₂—CH₃ |
| B-301 | OCH₂—C(CH₂—CH₃)═CH—CH₂—CH₃ |
| B-302 | OCH₂—CH(CH₂—CH₃)—CH═CH—CH₃ |
| B-303 | OCH₂—CH(CH₂—CH₃)—CH—CH═CH₂ |
| B-304 | OC(═CH—CH₃)—CH₂—CH₂—CH₂—CH₃ |
| B-305 | OCH(CH═CH₂)—CH₂—CH₂—CH₂—CH₃ |
| B-306 | OC(CH₂—CH₃)═CH—CH₂—CH₂—CH₃ |
| B-307 | OCH(CH₂—CH₃)—CH═CH—CH₂—CH₃ |
| B-308 | OCH(CH₂—CH₃)—CH₂—CH═CH—CH₃ |
| B-309 | OCH(CH₂—CH₃)—CH₂—CH₂—CH═CH₂ |
| B-310 | OC(═CH—CH₂—CH₃)—CH₂—CH₂—CH₃ |
| B-311 | OC(CH═CH—CH₃)—CH₂—CH₂—CH₃ |
| B-312 | OC(CH₂—CH═CH₂)—CH₂—CH₂—CH₃ |
| B-313 | OCH═C(CH₃)—C(CH₃)₃ |
| B-314 | OCH₂—C(═CH₂)—C(CH₃)₃ |
| B-315 | OCH₂—C(CH₃)₂—CH(═CH₂)—CH₃ |
| B-316 | OC(═CH₂)—CH(CH₃)—CH(CH₃)—CH₃ |
| B-317 | OC(CH₃)═C(CH₃)—CH(CH₃)—CH₃ |
| B-318 | OCH(CH₃)—C(═CH₂)—CH(CH₃)—CH₃ |
| B-319 | OCH(CH₃)—C(CH₃)═C(CH₃)—CH₃ |
| B-320 | OCH(CH₃)—CH(CH₃)—C(═CH₂)—CH₃ |
| B-321 | OC(CH₃)₂—CH═C(CH₃)—CH₃ |
| B-322 | OC(CH₃)₂—CH₂—C(═CH₂)—CH₃ |
| B-323 | OC(CH₃)₂—C(═CH₂)—CH₂—CH₃ |
| B-324 | OC(CH₃)₂—C(CH₃)═CH—CH₃ |
| B-325 | OC(CH₃)₂—CH(CH₃)CH═CH₂ |
| B-326 | OCH(CH₂—CH₃)—CH₂—CH(CH₃)—CH₃ |
| B-327 | OCH(CH₂—CH₃)—CH(CH₃)—CH₂—CH₃ |
| B-328 | OC(CH₃)(CH₂—CH₃)—CH₂—CH₂—CH₃ |
| B-329 | OCH(i-C₃H₇)—CH₂—CH₂—CH₃ |
| B-330 | OCH═C(CH₂—CH₃)—CH(CH₃)—CH₃ |
| B-331 | OCH₂—C(═CH—CH₃)—CH(CH₃)—CH₃ |
| B-332 | OCH₂—CH(CH═CH₂)—CH (CH₃)—CH₃ |
| B-333 | OCH₂—C(CH₂—CH₃)═C(CH₃)—CH₃ |
| B-334 | OCH₂—CH(CH₂—CH₃)—C(═CH₂)—CH₃ |
| B-335 | OCH₂—C(CH₃)(CH═CH₂)—CH₂—CH₃ |
| B-336 | OC(═CH₂)—CH (CH₂—CH₃)—CH₂—CH₃ |
| B-337 | OC(CH₃)═C(CH₂—CH₃)—CH₂—CH₃ |
| B-338 | OCH(CH₃)—C(═CH—CH₃)—CH₂—CH₃ |
| B-339 | OCH(CH₃)—CH(CH═CH₂)—CH₂—CH₃ |
| B-340 | OCH═C(CH₂—CH₃)—CH(CH₃)—CH₃ |
| B-341 | OCH₂—C(═CH—CH₃)—CH(CH₃)—CH₃ |
| B-342 | OCH₂—CH(CH═CH₂)—CH(CH₃)—CH₃ |
| B-343 | OCH₂—C(CH₂—CH₃)═C(CH₃)—CH₃ |
| B-344 | OCH₂—CH(CH₂—CH₃)—C(═CH₂)—CH₃ |
| B-345 | OC(═CH—CH₃)—CH₂—CH(CH₃)—CH₃ |
| B-346 | OCH(CH═CH₂)—CH₂—CH(CH₃)—CH₃ |
| B-347 | OC(CH₂—CH₃)═CH—CH(CH₃)—CH₃ |
| B-348 | OCH(CH₂—CH₃)CH═C(CH₃)—CH₃ |
| B-349 | OCH(CH₂—CH₃)CH₂—C(═CH₂)—CH₃ |
| B-350 | OC(═CH—CH₃)CH(CH₃)—CH₂—CH₃ |
| B-351 | OCH(CH═CH₂)CH(CH₃)—CH₂—CH₃ |
| B-352 | OC(CH₂—CH₃)═C(CH₃)—CH₂—CH₃ |
| B-353 | OCH(CH₂—CH₃)—C(═CH₂)—CH₂—CH₃ |
| B-354 | OCH(CH₂—CH₃)—C(CH₃)═CH—CH₃ |
| B-355 | OCH(CH₂—CH₃)—CH(CH₃)—CH═CH₂ |
| B-356 | OC(CH₃)(CH═CH₂)—CH₂—CH₂—CH₃ |
| B-357 | OC(CH₃)(CH₂—CH₃)—CH═CH—CH₃ |
| B-358 | OC(CH₃)(CH₂—CH₃)—CH₂—CH═CH₂ |
| B-359 | OC[═C(CH₃)—CH₃]—CH₂—CH₂—CH₃ |
| B-360 | OCH[C(═CH₂)—CH₃]—CH₂—CH₂—CH₃ |
| B-361 | OC(i-C₃H₇)═CH—CH₂—CH₃ |
| B-362 | OCH(i-C₃H₇)—CH═CH—CH₃ |
| B-363 | OCH(i-C₃H₇)—CH₂—CH═CH₂ |
| B-364 | OC(═CH—CH₃)—C(CH₃)₃ |
| B-365 | OCH(CH═CH₂)—C(CH₃)₃ |
| B-366 | OC(CH₃)(CH═CH₂)CH(CH₃)—CH₃ |
| B-367 | OC(CH₃)(CH₂—CH₃)C(═CH₂)—CH₃ |
| B-368 | O—(2—CH₃—Cyclohex-1-enyl) |
| B-369 | O—(2—(═CH₂)-c-C₆H₉) |
| B-370 | O-(2-CH₃-Cyclohex-2-enyl) |
| B-371 | O-(2-CH₃-Cyclohex-3-enyl) |
| B-372 | O-(2-CH₃-Cyclohex-4-enyl) |
| B-373 | O-(2-CH₃-Cyclohex-5-enyl) |
| B-374 | O-(2-CH₃-Cyclohex-6-enyl) |
| B-375 | O-(3-CH₃-Cyclohex-1-enyl) |
| B-376 | O-(3-CH₃-Cyclohex-2-enyl) |
| B-377 | O-(3-(═CH₂)-c-C₆H₉) |
| B-378 | O-(3-CH₃-Cyclohex-3-enyl) |
| B-379 | O-(3-CH₃-Cyclohex-4-enyl) |
| B-380 | O-(3-CH₃-Cyclohex-5-enyl) |
| B-381 | O-(3-CH₃-Cyclohex-6-enyl) |
| B-382 | O-(4-CH₃-Cyclohex-1-enyl) |
| B-383 | O-(4-CH₃-Cyclohex-2-enyl) |
| B-384 | O-(4-CH₃-Cyclohex-3-enyl) |
| B-385 | O-(4-(═CH₂)-c-C₆H₉) |

The compounds of the formula I or I′ and I″ and/or their agriculturally acceptable salts are suitable as active compounds, in particular as fungicides. They are distinguished by excellent activity against a broad spectrum of phytopathogenic fungi, in particular from the class of the Ascomycetes, Basidiomycetes, Deuteromycetes and Peronosporomycetes (syn. Oomycetes). Some of them are systemically active and can be used in crop protection as foliar fungicides, as fungicides for seed dressing and as soil fungicides.

They are particularly important in the control of a multitude of fungi on various cultivated plants, such as wheat, rye, barley, oats, rice, corn, grass, bananas, coton, soybeans, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on vegetables, oilseed rape, sugar beet and fruit and rice, such as, for example, A. solani or A. alternata on potatoes and tomatoes;
Aphanomyces species on sugar beet and vegetables;
Ascochyta species on cereals and vegetables;
Bipolaris and Drechslera species on corn, cereals, rice and lawns, such as, for example, D. maydis on corn;
Blumeria graminis (powdery mildew) on cereals;
Botrytis cinerea (gray mold) on strawberries, vegetables, flowers and grapevines;
Bremia lactucae on lettuce;
Cercospora species on corn, soybeans, rice and sugar beet;
Cochliobolus species on corn, cereals, rice, such as, for example Cochliobolus sativus on cereals, Cochliobolus miyabeanus on rice;
Colletotricum species on soybeans and cotton;
Drechslera species, Pyrenophora species on corn, cereals, rice and lawns, such as, for example, D. teres on barley or D. tritici-repentis on wheat;

Esca on grapevines, caused by Phaeoacremonium chlamydosporium, Ph. Aleophilum and Formitipora punctata (syn. Phellinus punctatus);
Exserohilum species on corn;
Erysiphe cichoracearum and Sphaerotheca fuliginea on cucumbers;
Fusarium and Verticillium species on various plants, such as, for example, F graminearum or F. culmorum on cereals or F. oxysporum on a multitude of plants, such as, for example, tomatoes;
Gaeumanomyces graminis on cereals;
Gibberella species on cereals and rice (for example Gibberella fujikuroi on rice);
Grainstaining complex on rice;
Helminthosporium species on corn and rice;
Michrodochium nivale on cereals;
Mycosphaerella species on cereals, bananas and groundnuts, such as, for example, M. graminicola on wheat or M. fijiensis on bananas;
Peronospora species on cabbage and bulbous plants, such as, for example, P. brassicae on cabbage or P. destructor on onions;
Phakopsara pachyrhizi and Phakopsara meibomiae on soybeans;
Phomopsis species on soybeans and sunflowers;
Phytophthora infestans on potatoes and tomatoes;

Phytophthora species on various plants, such as, for example, P. capsici on bell pepper;
Plasmopara viticola on grapevines;
Podosphaera leucotricha on apples;
Pseudocercosporella herpotrichoides on cereals;
Pseudoperonospora on various plants, such as, for example, P. cubensis on cucumber or P. humili on hops;
Puccinia species on various plants, such as, for example, P. triticina, P. striformins, P. hordei or P. graminis on cereals or P. asparagi on asparagus;
Pyricularia oryzae, Corticium sasakii, Sarociadium oryzae, S. attenuatum, Entyloma oryzae on rice;
Pyricularia grisea on lawns and cereals;
Pythium spp. on lawns, rice, corn, cotton, oilseed rape, sunflowers, sugar beet, vegetables and other plants, such as, for example, P. ultiumum on various plants, P. aphanidermatum on lawns;
Rhizoctonia species on cotton, rice, potatoes, lawns, corn, oilseed rape, potatoes, sugar beet, vegetables and on various plants, such as, for example,
R. solani on beet and various plants;
Rhynchosporium secalis on barley, rye and triticale;
Sclerotinia species on oilseed rape and sunflowers;
Septoria tritici and Stagonospora nodorum on wheat;
Erysiphe (syn. Uncinula) necator on grapevines;
Setospaeria species on corn and lawns;

Sphacelotheca reilinia on corn;
Thievaliopsis species on soybeans and cogton;
Tilletia species on cereals;
Ustilago species on cereals, corn and sugar cane, such as, for example, U. maydis on corn;
Venturia species (scab) on apples and pears, such as, for example, V. inaequalis on apples.

The compounds I are furthermore suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

In addition, the compounds according to the invention can also be used in crops which, owing to breeding including genetical engineering, are tolerant to attack by insects or fungi.

The present invention furthermore provides the use of the compounds according to the invention and/or their agriculturally acceptable salts for controlling phytopathogenic fungi. The present invention also provides the use of the compounds according to the invention and/or their agriculturally acceptable salts for controlling animal pests.

The compounds according to the invention and/or their agriculturally acceptable salts are employed by treating the fungi or the plants, seeds, materials or soil to be protected from fungal attack with a fungicidally effective amount of these compounds. The application can be carried out both before and after the infection of the materials, plants or seeds by the fungi.

Accordingly, the present invention furthermore provides a method for controlling phytopathogenic fungi wherein the fungi and/or the materials, plants, solid and/or seeds to be protected against fungal attack are treated with an effective amount of at least one pyrimidine according to the invention and/or an agriculturally acceptable salts thereof. The present invention furthermore provides a method for controlling animal pests in agriculture wherein the pests and/or the materials, plants, solid and/or seeds to be protected against them are treated with an effective amount of at least one pyrimidine according to the invention and/or an agriculturally acceptable salts thereof.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In the treatment of seed, the amounts of active compound employed are generally from 1 to 1000 g/100 kg of seed, preferably from 1 to 200 g/100 kg, in particular from 5 to 100 g/100 kg. Seed can be treated by methods known to the person skilled in the art, such as, for example, seed dressing, seed coating, seed dusting, seed soaking and seed pelleting.

Accordingly, the present invention furthermore provides seed comprising a compound according to the invention in an amount of from 1 to 1000 g per 100 kg. When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The present invention furthermore provides a composition, in particular a fungicidal composition, which comprises at least one compound according to the invention and/or an agriculturally acceptable salt thereof and at least one solid or liquid carrier. The composition may furthermore comprise at least one further fungicidally, insecticidally and/or herbicidally active compound, as illustrated in more detail below.

The compounds according to the invention and/or their agriculturally acceptable salts can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gammabutyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example finely divided silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

Formulations for seed treatment may further comprise binders and/or gelants and if appropriate dyes.

Binders can be added to increase the adhesion of the active compounds to the seed after the treatment. Suitable binders are for example EO/PO block copolymer surfactants, but also polyvinyl alcohols, polyvinylpyrrolidones, polyacrylates, polymethacrylates, polybutenes, polyisobutylenes, polystyrenes, polyethyleneamines, polyethyleneamides, polyethyleneimines (Lupasol®, Polymin®), polyethers, polyurethanes, polyvinyl acetates, Tylose and copolymers of these polymers. A suitable gelant is for example carrageen (Satiagel®).

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The concentrations of active compound in the ready-to-use preparations can be varied within relatively wide ranges. In general, they are between 0.0001 and 10%, preferably between 0.01 and 1%.

The active compounds can also be used with great success in the ultra-low-volume (ULV) process, it being possible to apply formulations with more than 95% by weight of active compound or even the active compound without additives. For the treatment of seed, the formulations in question give, after two-to-tenfold dilution, active compound concentrations of from 0.01 to 60% by weight, preferably from 0.1 to 40% by weight, in the ready-to-use preparations.

The following are examples of formulations according to the invention: 1. Products for dilution with water

### A Water-Soluble Concentrates (SL, LS)

10 parts by weight of a compound according to the invention are dissolved with 90 parts by weight of water or with a water-soluble solvent. As an alternative, wetters or other auxiliaries are added. The active compound dissolves upon dilution with water. This gives a formulation having an active compound content of 10% by weight.

### B Dispersible Concentrates (DC)

20 parts by weight of a compound according to the invention are dissolved in 70 parts by weight of cyclohexanone with addition of 10 parts by weight of a dispersant, for example polyvinylpyrrolidone. Dilution with water gives a dispersion. The active cornpound content is 20% by weight

### C Emulsifiable Concentrates (EC)

15 parts by weight of a compound according to the invention are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D Emulsions (EW, EO, ES)

25 parts by weight of a compound according to the invention are dissolved in 35 parts by weight of xylene with addition of calcium dodecylbenzenesulfonate and castor oil ethoxylate (in each case 5 parts by weight). This mixture is added to 30 parts by weight of water by means of an emulsifying machine (e.g. Ultraturrax) and made into a homogeneous emulsion. Dilution with water gives an emulsion. The formulation has an active compound content of 25% by weight.

### E Suspensions (SC, OD, FS)

In an agitated ball mill, 20 parts by weight of a compound according to the invention are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules (WG, SG)

50 parts by weight of a compound according to the invention are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP, SS, WS)

75 parts by weight of a compound according to the invention are ground in a rotorstator mill with addition of 25 parts by weight of dispersants, wetters and silica gel. Dilution with water gives a stable dispersion or solution of the active compound. The active compound content of the formulation is 75% by weight.

### 2. Products to be Applied Undiluted

H Dustable powders (DP, DS)

5 parts by weight of a compound according to the invention are ground finely and mixed intimately with 95 parts by weight of finely divided kaolin. This gives a dustable product with an active compound content of 5% by weight.

I Granules (GR, FG, GG, MG)

0.5 part by weight of a compound according to the invention is ground finely and associated with 99.5 parts by weight of carriers. Current methods are extrusion, spraydrying or the fluidized bed. This gives granules with an active compound content of 0.5% by weight to be applied undiluted.

J ULV solutions (UL)

10 parts by weight of a compound according to the invention are dissolved in 90 parts by weight of an organic solvent, for example xylene. This gives a product with an active compound content of 10% by weight to be applied undiluted.

Seed treatment typically utilizes water-soluble concentrates (LS), suspensions (FS), dusts (DS), water-dispersible and water-soluble powders (WS, SS), emulsions (ES), emulsifiable concentrates (EC) and gel formulations (GF). These formulations can be applied neat or preferably diluted to the seed. The application can take place prior to sowing.

Preference is given to using FS formulations for seed treatment. Such formulations typically comprise from 1 to 800 g/l of active compound, from 1 to 200 g/l of surfactants, from 0 to 200 g/l of antifreeze, from 0 to 400 g/l of binder, from 0 to 200 g/l of dyes and solvent, preferably water.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetting agent, tackifier, dispersant or emulsifier. Alternatively, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:100 to 100:1, preferably 1:10 to 10:1.

Suitable adjuvants in this sense are in particular: organically modified polysiloxanes, for example Break Thru S 240®; alcohol alkoxylates, for example Atplus 245®, Atplus MBA 1303®, Plurafac LF 300® and Lutensol ON 30®; EO/PO block polymers, for example Pluronic RPE 2035® and Genapol B®; alcohol ethoxylates, for example Lutensol XP 80®; and sodium dioctylsulfosuccinate, for example Leophen RA®.

The compositions according to the invention can, in the application form as fungicides, also be present together with other active compounds, for example with herbicides, pesticides (such as insecticides and acaricides), growth regulators, fungicides or also with fertilizers. When mixing the compounds according to the invention or the compositions comprising them with one or more further active compounds, in particular fungicides, it is in many cases possible to broaden the activity spectrum or to prevent the development of resistance. In many cases, synergistic effects are obtained.

The invention furthermore provides a combination of at least one pyrimidine I according to the invention and/or an agriculturally acceptable salt thereof and at least one further fungicidally, insecticidally, herbicidally and/or growth-regulating active compound.

The invention also provides a pesticidal composition comprising at least one pyrimidine I, in particular a pyrimidine of the formula I described in the above description as being preferred, and/or ein agriculturally acceptable salt thereof and at least one solid or liquid carrier. Such a pesticidal composition may comprise at least one further fungicidally, insecticidally and/or herbicidally active compound.

The following list of fungicides, together with which the compounds according to the invention may be used, is meant to illustrate the combination possibilities, but not to limit them:

### 1. Strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho((2,5-dimethylphenyloxymethylene)phenyl)-3-methoxyacrylate;

### 2. Carboxamides

carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluoro-biphenyl-2-yl)-3-difluoromethyl-1-methylpyrazole-4-carboxamide, N-(2-cyanophenyl)-3,4-dichloroisothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)ethyl)-2-ethanesulfonylamino-3-methylbutyramide;

### 3. Azoles

triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;

### 4. Nitrogenous Heterocyclyl Compounds:

pyridines: fluazinam, pyrifenox, 3-[5-(4-chlorophenyl)-2,3-dimethylisoxazolidin-3-yl]pyridine;
pyrimidines: bupirimate, cyprodinil, ferimzone, fenarimol, mepanipyrim, nuarimol, pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-5-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propylchromen-4-one, N,N-dimethyl-3-(3-bromo-6-fluoro-2-methylindole-1-sulfonyl)-[1,2,4]triazole-1-sulfonamide;

### 5. Carbamates and Dithiocarbamates

dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;

### 6. Other Fungicides

guanidines: dodine, iminoctadine, guazatine;
antibiotics: kasugamycin, polyoxins, streptomycin, validamycin A;
organometal compounds: fentin salts;
sulfur-containing heterocyclyl compounds: isoprothiolane, dithianon;
organophosphorous compounds: edifenphos, fosetyl, fosetyl-aluminum, iprobenfos, pyrazophos, tolclofos-methyl, phosphorous acid and its salts;
organochlorine compounds: thiophanate methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxyide, copper oxychloride, basic copper sulfate, sulfur;
others: spiroxamine, cyflufenamid, cymoxanil, metrafenone.
The present invention furthermore relates to the pharmaceutical use of the pyrimidines I according to the invention, in particular the pyrimidines of the formula I described in the description above as being preferred, and/or the pharmaceutically acceptable salts thereof, in particular their use for controlling tumors in mammals such as, for example, humans.

### SYNTHESIS EXAMPLES

With appropriate modification of the starting materials, the procedures given in the synthesis examples below were used to obtain further compounds according to the invention:

### Synthesis Example 1

### 4-Chloro-2-cyano-6-(2-methylpyrrolidin-1-yl)-5-trimethylsilylethynylpyrimidine (Table C, No. C-8)

### a) 4-Chloro-6-(2-methylpyrrolidin-1-yl)-2-methylthiopyrimidine

With stirring at 0-5° C., a mixture of 9.0 g (106 mmol) of 2-methylpyrrolidone and 15.2 g (150 mmol) of triethylamine was added dropwise to 19.5 g (100 mmol) of 4,6-dichloro-2-methylthiopyrimidine in 100 ml of methylene chloride, and the mixture was stirred for 1 d at room temperature. The reaction mixture was concentrated under reduced pressure and partitioned between water and methyl tert-butyl ether, and the organic phase was washed twice with water, dried over sodium sulfate and concentrated under reduced pressure. Yield 24.0 g.

### b) 5-Bromo-4-chlor-6-(2-methylpyrrolidin-1-yl)-2-methylthiopyrimidine

8.89 g (108.3 mmol) of sodium acetate were added to 24.0 g (98.5 mmol) of 4-chloro-2-methylthio-6-(2-methylpyrrolidin-1-yl)pyrimidine in 100 ml of acetic acid. With stirring at 18-25° C., 16.05 g (100.4 mmol) of bromine were added dropwise to the clear solution. The suspension was stirred at room temperature for 20 h, and another 3 drops of bromine were then added to bring the reaction to completion. The reaction mixture was poured into 1 l of ice-water, the mixture was stirred for 1 h and the precipitate was filtered off with suction, washed with water and dried under reduced pressure. Yield 29.0 g.

### c) 5-Bromo-4-chloro-6-(2-methylpyrrolidin-1-yl)-2-methylsulfonylpyrimidine

With stirring at 0-5° C., 44.3 g (179.8 mmol) of 70% strength 3-chloroperbenzoic acid were added to 29.0 g (89.9 mmol) of 5-bromo-4-chloro-2-methylthio-6-(2-methylpyrrolidin-1-yl)pyrimidine in 270 ml of methylene chloride, and the mixture was stirred at 5° C. for 7 h and then at room temperature for 2 d. Excess peroxide was destroyed using sodium thiosulfate and the reaction mixture was concentrated under reduced pressure, suspended in 250 ml of ethyl acetate, washed four times with in each case 50 ml of saturated sodium bicarbonate solution, dried over sodium sulfate and concentrated under reduced pressure. Yield 31.7 g.

### d) 5-Bromo-4-chloro-2-cyano-6-(2-methylpyrrolidin-1-yl)pyrimidine

At room temperature and with stirring, 8.73 g (134.1 mmol) of potassium cyanide and 228 mg (0.86 mmol) of crown ether (18-crown-6) were added to 31.7 g (89.4 mmol) of 5-bromo-4-chloro-6-(2-methylpyrrolidin-1-yl)-2-methylsulfonylpyrimidine in 180 ml of acetonitrile, and the mixture was stirred at room temperature for 7 d. The reaction mixture was concentrated under reduced pressure, taken up in 250 ml of ethyl acetate, washed three times with in each case 100 ml of water, dried over sodium sulfate and concentrated under reduced pressure and purified by chromatography on silica gel using cyclohexane/methyl tert-butyl ether. Yield 17.6 g.

### e) 4-Chloro-2-cyano-6-(2-methylpyrrolidin-1-yl)-5-trimethylsilylethynylpyrimidine

59.1 g (583 mmol) of triethylamine and 222 mg (1.17 mmol) of copper(1) iodide were added to 17.6 g (58.4 mmol) of 5-bromo-4-chloro-2-cyano-6-(2-methylpyrrolidin-1-yl)pyrimidine in 150 ml of acetonitril, and the mixture was warmed to 63° C. With stirring, 674 mg (0.58 mmol) of tetrakistriphenylphosphinepalladium(0) and 11.5 g (116.7 mmol) of trimethylsilylacetylene were then added, and the mixture was stirred at 65° C. for 7 h. After addition of a further 500 mg of tetrakistriphenylphosphinepalladium(0), the mixture was stirred for another 16 h at the same temperature. The reaction mixture was concentrated under reduced pressure and purified by chromatography on 330 g of silica gel using cyclohexane/methyl tert-butyl ether. Yield 11.0 g. ¹H-NMR (CDCl₃) δ=0.27 (s); 1.30 (d); 1.65-2.15 (m); 3.85-4.50 (br. m).

### Synthesis Example 2

### 4-Chloro-2-cyano-5-((E)-1,2-dibromo-2-trimethylsilylvinyl)-6-(2-methylpyrrolidin-1-yl)-pyrimidine (Table C, No. C-1)

With stirring at −12 to −5° C., 241 mg (1.51 mmol) of bromine were added dropwise to 400 mg (1.25 mmol) of 4-chloro-2-cyano-6-(2-methylpyrrolidin-1-yl)-5-trimethylsilylethynylpyrimidine in 4 ml of tetrachloromethane, and the mixture was stirred at −5° C. for 1 h. The reaction mixture was concentrated under reduced pressure and purified by chromatography on silica gel using cyclohexane/methyl tert-butyl ether. Yield 610 mg, m.p. 166-168° C.

### Synthesis Example 3

### 4-Chloro-2-cyano-5-((E)-2-chlor-1,2-dibromovinyl)-6-(2-methylpyrrolidin-1-yl)-pyrimidine (Table C, No. C-7)

With stirring at room temperature, 455 mg (7.83 mmol) of potassium fluoride and 907 mg (6.79 mmol) of N-chlorosuccinimide were added to 2.50 g (5.22 mmol) of 4-chloro-2-cyano-5-((E)-1,2-dibromo-2-trimethylsilylvinyl)-6-(2-methylpyrrolidin-1-yl)pyrimidine in 20 ml of 1,2-dichloroethane, and the mixture was then stirred at 80° C. for 64 h. To bring the reaction to completion, another 400 mg of N-chlorosuccinimide were then added and the mixture was stirred at 80° C. for 10 h. The reaction mixture was concentrated under reduced pressure and purified by chromatography on silica gel using cyclohexane/methyl tert-butyl ether. Yield 1.15 g. ¹H-NMR (CDCl₃) δ=1.25 (2 d, since the axially chiral substance is present as a diastereomer mixture); 1.70 (m); 1.90-2.20 (m); 3.40-3.80 (m); 4.60 (m).

### Synthesis Example 4

### N-Methoxy-4-chlor-5-((E)-2-chloro-1,2-dibromovinyl)-6-(2-methyl-pyrrolidin-1-yl)-2-pyrimidinecarboximidamide (Table C, No. C-4)

123 mg (0.11 mmol) of 5% strength sodium methoxide solution in methanol were added to 500 mg (1.13 mmol) of 4-chloro-2-cyano-5-((E)-2-chlor-1,2-dibromovinyl)-6-(2-methylopyrrolidin-1-yl)pyrimidine in 5 ml of methanol, and the mixture was stirred at room temperature for 16 h. Another 100 mg of 5% strength sodium methoxide solution in methanol were then added, and the mixture was stirred for a further 6 h. 114 mg (1.36 mmol) of O-methyl hydroxylamine hydrochloride were then added and the reaction mixture was stirred at room temperature for 3 d, concentrated under reduced pressure and partitioned between 15 ml of methyl tert-butyl ether and 12 ml of saturated sodium bicarbonate solution and the phases were separated. The aqueous phase was extracted with 20 ml of methyl tert-butyl ether and the combined organic phases were dried over sodium sulfate and concentrated under reduced pressure. The crude product was purified by chromatography on silica gel using cyclohexane/methyl tert-butyl ether. Yield 420 mg. ¹H-NMR (CDCl₃) δ=1.25 (2 d, since the axially chiral substance is present as a diastereomer mixture); 1.70 (m); 1.90-2.20 (m); 3.40-3.80 (m); 4.05 (s); 4.60 (m); 5.33 (br.).

TABLE C

| | |
|---|---|
| | I.A |
| 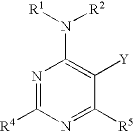 | |

| | | | | | | | | |
|---|---|---|---|---|---|---|---|---|
| R¹ | R² | R⁴ | R⁵ | Y | R⁶ | R⁷ | R⁸ | Physical data |

| | | | | | | | | |
|---|---|---|---|---|---|---|---|---|
| C-1 | —CH(CH₃)—(CH₂)₃— | CN | Cl | 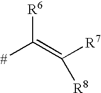 | Br | Si(CH₃)₃ | Br | m.p. 166-168° C. |
| C-2 | —CH(CH₃)—(CH₂)₃— | C(═NOH)NH₂ | Cl | 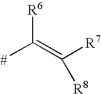 | Br | Si(CH₃)₃ | Br | m.p. 175-178° C. |
| C-3 | —CH(CH₃)—(CH₂)₃— | C(═O)NH₂ | Cl | 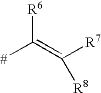 | Br | Cl | Br | m.p. 165-167° C. |
| C-4 | —CH(CH₃)—(CH₂)₃— | C(═NOCH₃)NH₂ | Cl | 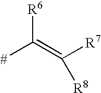 | Br | Cl | Br | ¹H-NMR(CDCl₃)δ = 1.25(2 ddiastereomer mixture);1.70(m);1.90-2.20(m);3.40-3.80(m);4.05(s); 4.60(m); 5.33(br.) |
| C-5 | —CH(CH₃)—(CH₂)₃— | CN | Cl | 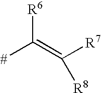 | Cl | Si(CH₃)₃ | Cl | |
| C-6 | —CH(CH₃)—(CH₂)₃— | CN | Cl | 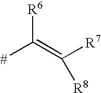 | Cl | Cl | Cl | |
| C-7 | —CH(CH₃)—(CH₂)₃— | CN | Cl | 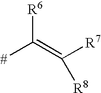 | Br | Cl | Br | ¹H-NMR(CDCl₃)δ = 1.25(2 d,diastereomer mixture);1.70(m);1.90-2.20(m);3.40-3.80(m); 4.60(m) |
| C-8 | —CH(CH₃)—(CH₂)₃— | CN | Cl | 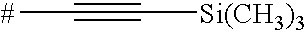 | — | — | — | ¹H-NMR(CDCl₃)δ = 0.27(s);1.30(d); 1.65-2.15(m);3.85-4.50(br. m) |
| C-9 | —CH(CH₃)—(CH₂)₃— | C(═O)NH₂ | Cl | 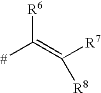 | Cl | Cl | Cl | m.p. 170-172° C. |
| C-10 | —CH(CH₃)—(CH₂)₃— | C(═NOCH₃)NH₂ | Cl | 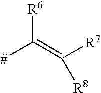 | Cl | Cl | Cl | m.p. 67-68° C. |
| C-11 | —CH(CH₃)—(CH₂)₃— | C(═NOH)NH₂ | Cl | 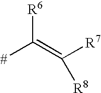 | C l | Cl | Cl | m.p. 174-175° C. |
| C-12 | —CH(CH₃)—(CH₂)₃— | C(═NOH)NH₂ | Cl | 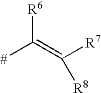 | Cl | Si(CH₃)₃ | Cl | m.p. 180-181° C. |
| C-13 | —CH(CH₃)—(CH₂)₃— | C(═NOH)NH₂ | Cl | 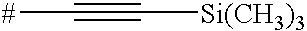 | — | — | — | m.p. 157-160° C. |
| C-14 | —CH(CH₃)—(CH₂)₃— | C(═NH)NH₂ | Cl | 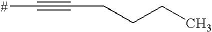 | — | — | — | m.p. 82–83° C. |
| C-15 | —CH(CH₃)—(CH₂)₃— | C(═NOH)NH₂ | Cl | 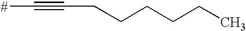 | — | — | — | m.p. 111–113° C. |
| C-16 | —CH(CH₃)—(CH₂)₃— | C(═O)NH₂ | CH₃ | 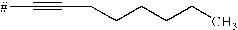 | — | — | — | m.p. 155-156° C. |
| C-17 | —CH(CH₃)—(CH₂)₃— | C(═NOH)NH₂ | CH₃ | 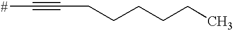 | — | — | — | m.p. 126-128° C. |

# denotes the point of attachment of the radical in question to the pyrimidine skeleton

### Examples of the Action Against Harmful Fungi

The active compounds, separately or together, were prepared as a stock solution with 25 mg of active compound which was made up to 10 ml with a mixture of acetone and/or DMSO and the emulsifier Uniperol® EL (wetting agent having emulsifying and dispersing action based on ethoxylated alkylphenols) in a volume ratio solvent/emulsifier of 99 to 1. The solution was then made up to 100 ml with water. This stock solution was diluted to the active compound concentration stated below using the solvent/emulsifier/water mixture described.

### Biological Example 1

### Curative Activity Against Brown Rust of Wheat Caused by Puccinia recondita

Leaves of potted wheat seedlings of the cultivar “Kanzler” were inoculated with a spore suspension of brown rust (Puccinia recondita). The pots were then placed into a chamber with high atmospheric humidity (90-95%) at 20-22° C. for 24 hours. During this time, the spores germinated and the germ tubes penetrated into the leaf tissue. The next day, the infected plants were sprayed to runoff point with the active compound solution described above having the concentration of active compound stated below. After the spray coating had dried on, the test plants were cultivated in a greenhouse at temperatures between 20 and 22° C. and at 65 to 70% relative atmospheric humidity for 7 days. The extent of the rust fungus development on the leaves was then determined.

The plants which had been treated with an application rate of 250 ppm of the compound No. C-3 showed an infection of 0%, whereas the untreated control plants were 90% infected.

### Biological Example 2

### Protective Activity Against Puccinia recondita on Wheat (Brown Rust of Wheat)

Leaves of potted wheat seedlings of the cultivar “Kanzler” were sprayed to runoff point with an aqueous suspension having the active compound concentration stated below. The next day, the treated plants were inoculated with a spore suspension of brown rust of wheat (Puccinia recondita). The plants were then placed in a chamber with high atmospheric humidity (90 to 95%) at 20 to 22° C. for 24 h. During this time, the spores germinated and the germ tubes penetrated into the leaf tissue. The next day, the test plants were returned to the greenhouse and cultivated at temperatures between 20 and 22° C. and 65 to 70% relative atmospheric humidity for a further 7 days. Tage kultiviert. The extent of the rust fungus development on the leaves was then determined visually.

The plants which had been treated with an application rate of 250 ppm of the compound No. C-3 showed an infection of 7%, whereas the untreated control plants were 90% infected.

For the micro test experiments below, the active compounds were formulated separately as a stock solution having a concentration of 10000 ppm in DMSO.

### Biological Example No. 3

### Activity Against the Rice Blast Pathogen Pyricularia oryzae in the Microtiter Test

The stock solution is pipetted onto a microtiter plate (MTP) and diluted to the stated active compound concentration using a malt-based aqueous nutrient medium for fungi. An aqueous spore suspension of Pyricularia oryzae was then added. The plates were placed in a water vapor-saturated chamber at temperatures of 18° C. Using an absorption photometer, the MTPs were measured at 405 nm on day 7 after the inoculation.

The measured parameters were compared to the growth of the active compound-free control variant and the fungus- and active compound-free blank value to determine the relative growth in % of the pathogens in the individual active compounds.

At 125 ppm of the compound No. C-1 or C-4, a relative pathogen growth of 0% was observed.

### Biological Example No. 4

### Activity Against the Late Blight Pathogen Phytophthora infestans in the Microtiter Test

The stock solution is pipetted onto a microtiter plate (MTP) and diluted to the stated active compound concentration using a pea juice-based aqueous nutrient medium for fungi. An aqueous zoospore suspension of Phytophthora infestans was then added. The plates were placed in a water vapor-saturated chamber at temperatures of 18° C. Using an absorption photometer, the MTPs were measured at 405 nm on day 7 after the inoculation.

The measured parameters were compared to the growth of the active compound-free control variant and the fungus- and active compound-free blank value to determine the relative growth in % of the pathogens in the individual active compounds.

At 125 ppm of the compound No. C-2, a relative pathogen growth of 10% was observed.

1-17. (canceled)

18. A method of controlling phytopathogenic fungi, comprising:
contacting said fungi, or plants, seeds, materials or soil to be protected from fungal attack with a fungicidally effective amount of a compound of formula I, 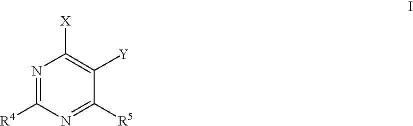
wherein:
X is NR¹R², OR³, SR³ or C₁-C₈-alkyl which may contain one, two, three or four identical or different substituents R^{a};
R¹, R² independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₄-C₈-cycloalkenyl, C₄-C₈-halocycloalkenyl, C₄-C₁₀-alkadienyl, C₄-C₁₀-haloalkadienyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkoxy, C₃-C₈-halocycloalkoxy, C₅-C₁₀-bicycloalkyl, NH₂, C₁-C₈-alkylamino, di-C₁-C₈-alkylamino, phenyl, naphthyl or a five- to six-membered saturated, partially unsaturated or aromatic heterocycle which contains one, two, three or four heteroatoms selected from the group consisting of O, N and S,
R¹ and R² together with the nitrogen atom to which they are attached may also form a five-, six- or seven-membered saturated or unsaturated heterocycle which may contain one, two or three further heteroatoms selected from the group consisting of O, N and S as ring members, wherein,
R¹ and/or R² or a heterocycle formed by R¹ and R² may carry one, two, three or four identical or different groups R^{a} and/or two substituents attached to the same or adjacent atoms or ring atoms may be C₁-C₆-alkylene, oxy-C₂-C₄-alkylene or oxy-C₁-C₃-alkyleneoxy;
R³ is C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₁-C₈-alkylamino, or di-C₁-C₈-alkylamino, where R³ may carry one, two, three or four identical or different groups R^{a};
R^{a} is halogen, cyano, nitro, hydroxy, carboxyl, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₄-C₁₀-alkadienyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₃-C₈-cycloalkenyloxy, C₅-C₁₀-bicycloalkyl, C₁-C₈-alkylcarbonyloxy, C₁-C₈-alkoxycarbonyloxy, aminocarbonyloxy, C₁-C₈-alkylaminocarbonyloxy, di-C₁-C₈-alkylaminocarbonyloxy, C₁-C₈-alkylthiocarbonyloxy, C₁-C₈-alkoxythiocarbonyloxy, aminothiocarbonyloxy, C₁-C₈-alkylaminothiocarbonyloxy, di-C₁-C₈-alkylaminothiocarbonyloxy, C₁-C₈-alkylaminothiocarbonyl, di-C₁-C₈-alkylaminothiocarbonyl, C₁-C₆-alkylene, oxyC₂-C₄-alkylene, oxy-C₁-C₃-alkyleneoxy, ═CH₂, ═CH(C₁-C₄-alkyl), ═C(C₁-C₄-alkyl)₂, —C(═O)-A, —C(═O)—O-A, —C(═O)—N(A′)A, C(A′)(=N—OA), N(A′)A, N(A′)C(═O)-A, N(A″)—C(═O)—N(A′)A, S(═O)ₘ-A, S(═O)ₘ—O-A, S(═O)ₘ—N(A′)A, Si(C₁-C₆-alkyl)₃, phenyl, naphthyl, or a five-, six-, seven-, eight-, nine- or ten-membered saturated, partially unsaturated or aromatic heterocycle which contains one, two, three or four heteroatoms selected from the group consisting of O, N and S;
wherein the aliphatic, alicyclic and/or aromatic groups in R^{a} for their part may carry one, two or three identical or different groups R^{b}:
R^{b} is halogen, cyano, nitro, hydroxy, mercapto, C₁-C₈-alkyl, C₂-C₈-alkenyl, C₂-C₈-alkynyl, C₄-C₁₀-alkadienyl, C₃-C₈-cycloalkyl, C₃-C₈-cycloalkenyl, C₅-C₁₀-bicycloalkyl, C₁-C₈-alkoxy, C₂-C₈-alkenyloxy, C₂-C₈-alkynyloxy, C₃-C₈-cycloalkoxy, C₃-C₈-cycloalkenyloxy, aminothiocarbonyl, C₁-C₈-alkyl-carbonyloxy, C₁-C₈-alkylaminothiocarbonyl, di-C₁-C₈-alkylaminothiocarbonyl, C₁-C₈-alkylthio, C₁-C₈-alkylsulfinyl, C₁-C₈-alkylsulfonyl, C₆-C₁₄-aryl, C₆-C₁₄-aryloxy, C₆-C₁₄-arylthio, C₆-C₁₄-aryl-C₁-C₆-alkoxy, C₆-C₁₄-aryl-C₁-C₆-alkyl, a five- to ten-membered saturated, partially unsaturated or aromatic heterocycle which contains one, two, three or four heteroatoms from the group consisting of O, N and S, heterocyclyloxy, heteroaryloxy, heteroarylthio, —C(═O)-A, —C(═O)—O-A, —C(═O)—N(A′)A, C(A′)(=N—OA), or N(A′)A; wherein the aliphatic, alicyclic or aromatic groups may be partially or fully halogenated and/or substituted by C₁-C₈-alkyl and/or C₁-C₈-haloalkyl groups;
m is 0, 1 or 2;
A, A′, A″ independently of one another are hydrogen, C₁-C₈-alkyl, C₂-C₈-alkenyl, C₂-C₈-alkynyl, C₃-C₈-cycloalkyl, C₃-C₈-cycloalkenyl, or phenyl, where the organic radicals may be partially or fully halogenated and/or may be mono- or polysubstituted by nitro, cyanato, cyano, C₁-C₄-alkoxy; A and A′ together with the atoms to which they are attached may also be a five- or six-membered saturated, partially unsaturated or aromatic heterocycle which contains one, two, three or four heteroatoms selected from the group consisting of O, N and S;
Y is (C₂-C₁₀)-alkenyl, (C₂-C₁₀)-alkynyl, (C₃-C₁₀)-cycloalkenyl, a five-, six-, seven-, eight-, nine- or ten-membered partially unsaturated heterocycle which contains one, two or three heteroatoms selected from the group consisting of O, N and S as ring members, or an imino group; wherein Y may carry one, two, three or four identical or different groups R^{u} and/or two substituents attached to the same or adjacent atoms or ring atoms are C₁-C₆-alkylene, oxy-C₂-C₄-alkylene or oxy-C₁-C₃-alkyleneoxy, wherein R^{u} is as defined below:
R^{u} independently of one another are halogen, cyano, nitro, C₁-C₈-alkyl, C₁-C₈-haloalkyl, C₁-C₈-alkoxy-C₁-C₈-alkyl, C₂-C₈-alkenyl, C₂-C₈-haloalkenyl, C₂-C₈-alkynyl, C₂-C₈-haloalkynyl, C₄-C₁₀-alkadienyl, C₃-C₆-cycloalkyl, C₃-C₆-halocycloalkyl, C₄-C₆-cycloalkenyl, C₄-C₆-halocycloalkenyl, C₅-C₁₀-bicycloalkyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkoxy, C₃-C₈-halocycloalkoxy, C₃-C₈-cycloalkenyloxy, C₁-C₈-alkylcarbonyloxy, C₁-C₈-alkylaminothiocarbonyl, di-C₁-C₈-alkylaminothiocarbonyl, C₁-C₆-alkylene, oxy-C₂-C₄-alkylene, oxy-C₁-C₃-alkyleneoxy, —C(═O)-A, —C(═O)—O-A, —C(═O)—N(A′)A, C(A′)(═N—OA), N(A′)A, N(A′)-C(═O)-A, N(A″)—C(═O)—N(A′)A, S(═O)ₘ-A, S(═O)ₘ—O-A, S(═O)ₘ—N(A′)A, —Si(C₁-C₆-alkyl)₃, phenyl, naphthyl, or a five- to ten-membered saturated, partially unsaturated or aromatic heterocycle which contains one, two, three or four heteroatoms selected from the group consisting of O, N and S; wherein m, A, A′, A″ are as defined above;
R^{u} may carry one, two, three or four identical or different groups R^{a}, where R^{a} is as defined above;
R⁴ is a five- or six-membered saturated, partially unsaturated or aromatic heterocycle which contains one, two, three or four heteroatoms selected from the group consisting of O, N and S, where R⁴ may be partially or fully halogenated and/or may carry one, two, three or four identical or different groups R^{v}:
R^{v} is halogen, cyano, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₁-C₆-alkoxy, C₁-C₆-haloalkoxy, C₃-C₆-cycloalkyl, C₃-C₆-halocycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, Oxo (═O), —C(═O)-A, —C(═O)—O-A, —C(═O)—N(A′)A, —C(A′)(═N—OA), N(A′)A, N(A′)-C(═O)A, N(A″-C(═O)—N(A′)A, —S(═O)ₘ-A, —S(═O)ₘ—O-A or —S(═O)ₘ—N(A′)A; where m, A, A′, A″ are as defined above;
R⁴ may furthermore be:
cyano, C(═W)OR^{c}, C(═W)NR^{z}R^{d}, C(═W)NR^{c}—NR^{z}R^{d}, C(═W)R^{c},
CR^{c}R^{d}OR^{z}, CR^{c}R^{d}—NR^{z}R^{f},
ON(═CR^{c}R^{d}), O—C(═W)R^{c},
NR^{c}R^{d}, NR^{c}C(═W)R^{d}), NR^{c}C(═W)OR^{d}), NR^{c}C(═W)—NR^{z}R^{d}), NR^{c}(N═CR^{f}R^{d}),
NR^{c}—NR^{z}R^{d}, or NR^{z}—OR^{c}; where
W is O, S, NR^{g}, NOR^{g} or N—NR^{y}R^{f};
R^{c}, R^{d}, R^{f}, R^{g} independently of one another are hydrogen, C₁-C₆-alkyl, C₂-C₆-alkenyl, C₂-C₆-alkynyl, C₃-C₆-cycloalkyl or C₄-C₆-cycloalkenyl;
R^{y}, R^{z} independently have the same meanings as R^{c} and may additionally be C(═O)—R^{g} or C(═O)—OR^{g};
where the aliphatic and/or alicyclic groups of the radical definitions of R^{c}, R^{d}, R^{f}, R^{g}, R^{y} and R^{z} for their part may be partially or fully halogenated and/or may carry one, two, three or four identical or different groups R^{w}:
R^{w} is halogen, cyano, C₁-C₈-alkyl, C₂-C₁₀-alkenyl, C₂-C₁₀-alkynyl, C₃-C₆-cycloalkyl, C₃-C₆-cycloalkenyl, C₁-C₆-alkoxy, C₂-C₁₀-alkenyloxy, C₂-C₁₀-alkynyloxy, C₃-C₆-cycloalkoxy or C₃-C₆-cycloalkenyloxy;
and where two of the radicals R^{c}, R^{d}, R^{f}, R^{y}, R^{z} together with the atoms to which they are attached may form a five- or six-membered saturated, partially unsaturated or aromatic heterocycle which contains one, two, three or four heteroatoms selected from the group consisting of O, N and S;
R⁵ is hydrogen, hydroxy, halogen, cyano, azido, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₄-C₈-alkadienyl, C₃-C₈-cycloalkyl, C₃-C₈-halocycloalkyl, C₁-C₈-alkoxy, C₁-C₈-haloalkoxy, C₂-C₈-alkenyloxy, C₃-C₄-alkynyloxy, C₁-C₈-alkylthio, C₁-C₈-alkylsulfinyl, C₁-C₈-alkylsulfonyl, amino, C₁-C₈-alkylamino, or di-(C₁-C₈-alkyl)amine, where the aliphatic and alicyclic groups of the radical definitions of R⁵ for their part may contain one, two, three or four substituents independently of one another selected from the group consisting of halogen, cyano, hydroxy, nitro, C₁-C₄-alkoxy, C₁-C₄-alkoxycarbonyl, amino, C₁-C₄-alkylamino and di-C₁-C₄-alkylamino;
or agriculturally acceptable salts thereof.

19. A compound of formula I, 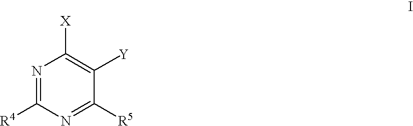
wherein:
X is NR¹R², OR³, SR³ or C₁-C₈-alkyl which may contain one, two, three or four identical or different substituents R^{a};
Y is (C₂-C₁₀)-alkenyl, (C₂-C₁₀)-alkynyl, (C₃-C₁₀)-cycloalkenyl, a five-, six-, seven-, eight-, nine- or ten-membered partially unsaturated heterocycle which contains one, two or three heteroatoms from the group consisting of O, N and S as ring members, or an imino group; where Y may carry one, two, three or four identical or different groups R^{u} and/or two substituents attached to the same or adjacent atoms or ring atoms are C₁-C₆-alkylene, oxy-C₂-C₄-alkylene or oxy-C₁-C₃-alkyleneoxy, where R^{u} is as defined below:
R^{u} independently of one another are halogen, cyano, nitro, C₁-C₈-alkyl, C₁-C₈-haloalkyl, C₁-C₈-alkoxy-C₁-C₈-alkyl, C₂-C₈-alkenyl, C₂-C₈-haloalkenyl, C₂-C₈-alkynyl, C₂-C₈-haloalkynyl, C₄-C₁₀-alkadienyl, C₃-C₆-cycloalkyl, C₃-C₆-halocycloalkyl, C₄-C₆-cycloalkenyl, C₄-C₆-halocycloalkenyl, C₅-C₁₀-bicycloalkyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkoxy, C₃-C₈-halocycloalkoxy, C₃-C₈-cycloalkenyloxy, C₁-C₈-alkylcarbonyloxy, C₁-C₈-alkylaminothiocarbonyl, di-C₁-C₈-alkylaminothiocarbonyl, C₁-C₆-alkylene, oxy-C₂-C₄-alkylene, oxy-C₁-C₃-alkyleneoxy, —C(═O)-A, —C(═O)—O-A, —C(═O)—N(A′)A, C(A′)(=N—OA), N(A′)A, N(A′)-C(═O)-A, N(A″)—C(═O)—N(A′)A, S(═O)ₘ-A, S(═O)ₘ—O-A, S(═O)ₘ—N(A′)A, —Si(C₁-C₆-alkyl)₃, phenyl, naphthyl, or a five- to ten-membered saturated, partially unsaturated or aromatic heterocycle which contains one, two, three or four heteroatoms selected from the group consisting of O, N and S; where m, A, A′, A″ are as defined above;
R^{u} may carry one, two, three or four identical or different groups R^{a}, where R^{a} is as defined above;
R¹ is C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₄-C₈-cycloalkenyl, C₄-C₈-halocycloalkenyl, C₄-C₁₀-alkadienyl, C₄-C₁₀-haloalkadienyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkoxy, C₃-C₈-halocycloalkoxy, C₅-C₁₀-bicycloalkyl, NH₂, C₁-C₈-alkylamino, di-C₁-C₈-alkylamino, phenyl, naphthyl or a five- to six-membered saturated, partially unsaturated or aromatic heterocycle which contains one, two, three or four heteroatoms from the group consisting of O, N and S,
R² is hydrogen or a group mentioned for R¹;
R¹ and R² together with the nitrogen atom to which they are attached may also form a five-, six- or seven-membered saturated or unsaturated heterocycle which may contain one, two or three further heteroatoms selected from the group consisting of O, N and S as ring members, where
R¹ and/or R² or a heterocycle formed by R¹ and R² may carry one, two, three or four identical or different groups R^{a} and/or two substituents attached to the same or adjacent atoms or ring atoms may be C₁-C₆-alkylene, oxy-C₂-C₄-alkylene or oxy-C₁-C₃-alkyleneoxy;
R³ is C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₈-cycloalkyl, C₃-C₈-halocycloalkyl, C₁-C₈-alkylamino, or di-C₁-C₈-alkylamino, where R³ may carry one, two, three or four identical or different groups R^{a};
R^{a} is halogen, cyano, nitro, hydroxy, carboxyl, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₄-C₁₀-alkadienyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkyl, C₃-C₈-halocycloalkyl, C₃-C₈-cycloalkenyl, C₃-C₈-cycloalkoxy, C₃-C₈-halocycloalkoxy, C₃-C₈-cycloalkenyloxy, C₅-C₁₀-bicycloalkyl, C₁-C₈-alkylcarbonyloxy, C₁-C₈-alkoxycarbonyloxy, aminocarbonyloxy, C₁-C₈-alkylaminocarbonyloxy, di-C₁-C₈-alkylaminocarbonyloxy, C₁-C₈-alkylthiocarbonyloxy, C₁-C₈-alkoxythiocarbonyloxy, aminothiocarbonyloxy, C₁-C₈-alkylaminothiocarbonyloxy, di-C₁-C₈-alkylaminothiocarbonyloxy, C₁-C₈-alkylaminothiocarbonyl, di-C₁-C₈-alkylaminothiocarbonyl, C₁-C₆-alkylene, oxyC₂-C₄-alkylene, oxy-C₁-C₃-alkyleneoxy, ═CH₂, ═CH(C₁-C₄-alkyl), ═C(C₁-C₄-alkyl)₂, —C(═O)-A, —C(═O)—O-A, —C(═O)—N(A′)A, C(A′)(=N—OA), N(A′)A, N(A′)C(═O)-A, N(A″)—C(═O)—N(A′)A, S(═O)ₘ-A, S(═O)ₘ—OA, S(═O)ₘ—N(A′)A, Si(C₁-C₆-alkyl)₃, phenyl, naphthyl, or a five-, six-, seven-, eight-, nine- or ten-membered saturated, partially unsaturated or aromatic heterocycle which contains one, two, three or four heteroatoms selected from the group consisting of O, N and S;
wherein the aliphatic, alicyclic and/or aromatic groups in R^{a} for their part may carry one, two or three identical or different groups R^{b}:
R^{b} is halogen, cyano, nitro, hydroxy, mercapto, C₁-C₈-alkyl, C₂-C₈-alkenyl, C₂-C₈-alkynyl, C₄-C₁₀-alkadienyl, C₃-C₈-cycloalkyl, C₃-C₈-cycloalkenyl, C₅-C₁₀-bicycloalkyl, C₁-C₈-alkoxy, C₂-C₈-alkenyloxy, C₂-C₈-alkynyloxy, C₃-C₈-cycloalkoxy, C₃-C₈-cycloalkenyloxy, aminothiocarbonyl, C₁-C₈-alkyl-carbonyloxy, C₁-C₈-alkylaminothiocarbonyl, di-C₁-C₈-alkylaminothiocarbonyl, C₁-C₈-alkylthio, C₁-C₈-alkylsulfinyl, C₁-C₈-alkylsulfonyl, C₆-C₁₄-aryl, C₆-C₁₄-aryloxy, C₆-C₁₄-arylthio, C₆-C₁₄-aryl-C₁-C₆-alkoxy, C₆-C₁₄-aryl-C₁-C₆-alkyl, a five- to ten-membered saturated, partially unsaturated or aromatic heterocycle which contains one, two, three or four heteroatoms from the group consisting of O, N and S, heterocyclyloxy, heteroaryloxy, heteroarylthio, —C(═O)-A, —C(═O)—O-A, —C(═O)—N(A′)A, C(A′)(=N—OA), or N(A′)A; wherein the aliphatic, alicyclic or aromatic groups may be partially or fully halogenated and/or substituted by C₁-C₈-alkyl and/or C₁-C₈-haloalkyl groups;
m is 0, 1 or 2;
A, A′, A″ independently of one another are hydrogen, C₁-C₈-alkyl, C₂-C₈-alkenyl, C₂-C₈-alkynyl, C₃-C₈-cycloalkyl, C₃-C₈-cycloalkenyl, or phenyl, where the organic radicals may be partially or fully halogenated and/or may be mono- or polysubstituted by nitro, cyanato, cyano, C₁-C₄-alkoxy; A and A′ together with the atoms to which they are attached may also be a five- or six-membered saturated, partially unsaturated or aromatic heterocycle which contains one, two, three or four heteroatoms selected from the group consisting of O, N and S;
R⁴ is a five- or six-membered saturated, partially unsaturated or aromatic heterocycle which contains one, two, three or four heteroatoms selected from the group consisting of O, N and S, where R⁴ may be partially or fully halogenated and/or may carry one, two, three or four identical or different groups R^{v};
R⁴ may furthermore be:
cyano, C(═W)OR^{c}, C(═W)NR^{z}R^{d}, C(═W)NR^{c}—NR^{z}R^{d}, C(═W)R^{c},
CR^{c}R^{d}—OR^{z}, CR^{c}R^{d}—NR^{z}R^{f},
ON(═CR^{c}R^{d}), O—C(═W)R^{c}
NR^{c}C(═W)R^{d}), NR^{c}C(═W)OR^{d}), NR^{c}C(═W)—NR^{z}R^{d}), NR^{c}(N═CR^{f}R^{d}),
NR^{c}—NR^{z}R^{d}, or NR^{z}—OR^{c}; where
W is S, NR^{g}, NOR^{g} or N—NR^{y}RF;
R^{v} is halogen, cyano, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₁-C₆-alkoxy, C₁-C₆-haloalkoxy, C₃-C₆-cycloalkyl, C₃-C₆-halocycloalkyl, C₂-C₈-alkenyloxy, C₂-C₈-alkynyloxy, C₄-C₆-cycloalkenyl, C₃-C₆-cycloalkyloxy, C₄-C₆-cycloalkenyloxy, Oxo (═O), —C(═O)-A, —C(═O)—O-A, —C(═O)—N(A′)A, —C(A′)(=N—OA), N(A′)A, N(A′)-C(═O)A, N(A″-C(═O)—N(A′)A, —S(═O)ₘ-A, —S(═O)ₘ—O-A or —S(═O)ₘ—N(A′)A; where m, A, A′, A″ are as defined above;
R^{c}, R^{d}, R^{f}, R^{g} independently of one another are hydrogen, C₁-C₆-alkyl, C₂-C₆-alkenyl, C₂-C₆-alkynyl, C₃-C₆-cycloalkyl or C₄-C₆-cycloalkenyl;
R^{y}, R^{z} independently have the same meanings as R^{c} and may additionally be C(═O)—R^{g} or C(═O)—OR^{g};
where the aliphatic and/or alicyclic groups of the radical definitions of R^{c}, R^{d}, R^{f}, R^{g}, R^{y} and R^{z} for their part may be partially or fully halogenated and/or may carry one, two, three or four identical or different groups R^{w}:
R^{w} is halogen, cyano, C₁-C₈-alkyl, C₂-C₁₀-alkenyl, C₂-C₁₀-alkynyl, C₃-C₆-cycloalkyl, C₃-C₆-cycloalkenyl, C₁-C₆-alkoxy, C₂-C₁₀-alkenyloxy, C₂-C₁₀-alkynyloxy, C₃-C₆-cycloalkoxy or C₃-C₆-cycloalkenyloxy;
and where two of the radicals R^{c}, R^{d}, R^{f}, R^{y}, R^{z} together with the atoms to which they are attached may form a five- or six-membered saturated, partially unsaturated or aromatic heterocycle which contains one, two, three or four heteroatoms selected from the group consisting of O, N and S;
R⁵ is hydrogen, hydroxy, halogen, cyano, azido, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₄-C₈-alkadienyl, C₃-C₈-cycloalkyl, C₃-C₈-halocycloalkyl, C₁-C₈-alkoxy, C₁-C₈-haloalkoxy, C₂-C₈-alkenyloxy, C₃-C₄-alkynyloxy, C₁-C₈-alkylthio, C₁-C₈-alkylsulfinyl, C₁-C₈-alkylsulfonyl, amino, C₁-C₈-alkylamino, or di-(C₁-C₈-alkyl)amine, where the aliphatic and alicyclic groups of the radical definitions of R⁵ for their part may contain one, two, three or four substituents independently of one another selected from the group consisting of halogen, cyano, hydroxy, nitro, C₁-C₄-alkoxy, C₁-C₄-alkoxycarbonyl, amino, C₁-C₄-alkylamino and di-C₁-C₄-alkylamino;
and wherein two of the radicals R^{c}, R^{d}, R^{f}, R^{y}, R^{z} together with the atoms to which they are attached may form a five- or six-membered saturated, partially unsaturated or aromatic heterocycle which contains one, two, three or four heteroatoms selected from the group consisting of O, N and S;
or agriculturally acceptable salts thereof.

20. The compound of claim 19, wherein R⁴ is not pyridin-2-yl.

21. The compound of claim 19, wherein R⁴ is not an aromatic six-membered heterocycle.

22. The compound of claim 19, wherein R⁴ is C(═W)OR^{c}, C(═W)NR^{z}R^{d}, C(═W)NR^{c}—NR^{z}R^{d}, C(═W)R^{c}, CR^{c}R^{d}—OR^{z}, CR^{c}R^{d}—NR^{z}R^{f}, NR^{c}C(═W)R^{d}), NR^{c}C(═W)OR^{d}), NR^{c}C(═W)—NR^{z}R^{d}) or NR^{z}—OR^{c}, where W is O or NOR^{g}, R^{c}, R^{d}, R^{f}, R^{z} independently of one another are hydrogen or C₁-C₆-alkyl and R^{z} may additionally be C(═O)—R^{g} or C(═O)—OR^{g}.

23. The compound of claim 19, wherein R⁴ is a five-membered saturated, partially unsaturated or aromatic heterocycle which contains one, two, three or four heteroatoms selected from the group consisting of O, N and S, wherein the heterocycle may be partially or fully halogenated and/or may carry one, two, three or four identical or different groups R^{v}.

24. The compound of claim 23, wherein said five-membered heterocycle is aromatic.

25. The compound of claim 19, wherein X is NR¹R².

26. The compound of claim 19, wherein R² is different from hydrogen.

27. The compound of claim 19, wherein Y is a group (A): 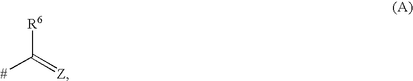
wherein, # is the point of attachment to the pyrimidine skeleton,
Z is CR⁷R⁸ or NR⁹
R⁶, R⁷, R⁸, R⁹ independently of one another are hydrogen, halogen, cyano, nitro, C₁-C₈-alkyl, C₁-C₈-haloalkyl, C₁-C₈-alkoxy-C₁-C₈-alkyl, C₂-C₈-alkenyl, C₂-C₈-haloalkenyl, C₂-C₈-alkynyl, C₂-C₈-haloalkynyl, C₄-C₁₀-alkadienyl, C₃-C₆-cycloalkyl, C₃-C₆-halocycloalkyl, C₄-C₆-cycloalkenyl, C₄-C₆-halocycloalkenyl, C₅-C₁₀-bicycloalkyl, C₁-C₈-alkoxy, C₁-C₈-haloalkoxy, C₂-C₈-alkenyloxy, C₂-C₈-haloalkenyloxy, C₂-C₈-alkynyloxy, C₂-C₈-haloalkynyloxy, C₃-C₈-cycloalkoxy, C₃-C₈-halocycloalkoxy, C₃-C₈-cycloalkenyloxy, C₁-C₈-alkylcarbonyloxy, C₁-C₈-alkylaminothiocarbonyl, di-C₁-C₈-alkylaminothiocarbonyl, —C(═O)-A, —C(═O)—O-A, —C(═O)—N(A′)A, C(A′)(=N—OA), N(A′)A, N(A′)-C(═O)-A, N(A″)—C(═O)—N(A′)A, S(═O)ₘ-A, S(═O)ₘ—O-A, S(═O)ₘ—N(A′)A, —Si(C₁-C₆-alkyl)₃, phenyl, naphthyl, or a five- to ten-membered saturated, partially unsaturated or aromatic heterocycle which contains one, two, three or four heteroatoms selected from the group consisting of O, N and S;
R⁶ and R⁷ or R⁶ and R⁹ together with the atoms to which they are attached may also form a five-, six-, seven-, eight-, nine- or ten-membered partially unsaturated cycle or partially unsaturated heterocycle which contains one, two or three heteroatoms selected from the group consisting of O, N and S as ring members;
R⁶, R⁷, R⁸ and/or a cycle or heterocycle formed by R⁶ and R⁷ or R⁶, R⁹ or a cycle or heterocycle formed by R⁶ and R⁹ may carry one, two, three or four identical or different groups R^{a}, where m, A, A′, A″ and R^{a} are as defined in claim 1.

28. The compound of 27, wherein Z is NR⁹.

29. The compound of claim 27, wherein Z is CR⁷R⁸.

30. A pesticidal composition comprising, at least one compound of claim 19 and/or an agriculturally acceptable salt thereof and at least one solid or liquid carrier.

31. The pesticidal composition according to claim 30 which comprises at least one further fungicidally, insecticidally and/or herbicidally active compound.

32. A seed comprising, a compound of claim 19 and/or an agriculturally acceptable salt thereof in an amount of from 1 to 1000 g per 100 kg.

33. A method of controlling phytopathogenic fungi, comprising:
contacting said fungi, or plants, seeds, materials or soil to be protected from fungal attack with a fungicidally effective amount of a compound of claim 19.
